SEQUENCE LISTING (110 sequences)

SEQ ID NO: 1 - DNA, 510 bp, Homo sapiens
tgaggctgccttataaagcaccaagaggctgccagtgggacattttctcggccctgccagcccccaggaggaaggtgggtctgaatctagcaccatgacggaactagagacagccatgggcatgatcatagacgtcttttcccgatattcgggcagcgagggcagcacgcagaccctgaccaagggggagctcaaggtgctgatggagaaggagctaccaggcttcctgcagagtggaaaagacaaggatgccgtggataaattgctcaaggacctggacgccaatggagatgcccaggtggacttcagtgagttcatcgtgttcgtggctgcaatcacgtctgcctgtcacaagtactttgagaaggcaggactcaaatgatgccctggagatgtcacagattcctggcagagccatggtcccaggcttcccaaaagtgtttgttggcaattattcccctaggctgagcctgctcatgtacctctgattaataaatgcttatgaaatga

SEQ ID NO: 2 - DNA, 1024 bp, Homo sapiens
gtcgcctctcacccgccccggccgctccagcccgaggcgccccgaccccgcgccactccgcgcccggccagccgcccgcagccatggggctcctgcccaagctcggcgtgtcccagggcagcgacacctctactagccgagccggccgctgtgcccgctcggtcttcggcaacattaaggtgtttgtgctctgccaaggcctcctgcagctctgccaactcctgtacagcgcctacttcaagagcagcctcaccaccattgagaagcgctttgggctctccagttcttcatcgggtctcatttccagcttgaatgagatcagcaatgccatcctcatcatctttgtcagctactttggcagccgggtgcaccgtccacgtctgattggcatcggaggtctcttcctggctgcaggtgccttcatcctcaccctcccacacttcctctccgagccctaccagtacaccttggccagcactgggaacaacagccgcttgcaggccgagctctgccagaagcattggcaggacctgcctcccagtaagtgccacagcaccacccagaacccccagaaggagaccagcagcatgtggggcctgatggtggttgcccagctgctggctggcatcgggacagtgcctattcagccatttgggatctcctatgtggatgacttctcagagcccagcaactcgcccctgtacatctccatcttatttgccatctctgtatttggaccggctttcgggtacctgctgggctctatcatgctgcagatctttgtggactatggcagggtcaacacagctgcagttaacttggtcccgggtgacccccgatggattggagcctggtggctaggcctgctcatttcttcagctttattggttctcacctctttccccttttttttcttccctcgagcaatgcccataggagcaaagagggctcctgccacagcagatgaagcaaggaagttggaggaggccaagtcaagaggctccctggtggatttcattaaacg

SEQ ID NO: 3 - DNA, 738 bp, Homo sapiens
gggactgcacatcctgggcctgcggcgcagtgtagacctgggaggatgggcggcctgctgctggctgcttttctggctttggtctcggtgcccagggcccaggccgtgtggttgggaagactggaccctgagcagcttcttgggccctggtacgtgcttgcggtggcctcccgggaaaagggctttgccatggagaaggacatgaagaacgtcgtgggggtggtggtgaccctcactccagaaaacaacctgcggacgctgtcctctcagcacgggctgggagggtgtgaccagagtgtcatggacctgataaagcgaaactccggatgggtgtttgagaatccctcaataggcgtgctggagctctgggtgctggccaccaacttcagagactatgccatcatcttcactcagctggagttcggggacgagcccttcaacaccgtggagctgtacagtctgacggagacagccagccaggaggccatggggctcttcaccaagtggagcaggagcctgggcttcctgtcacagtagcaggcccagctgcagaaggacctcacctgtgctcacaagatccttctgtgagtgctgcgtccccagtagggatggcgcccacagggtcctgtgacctcggccagtgtccacccacctcgctcagcggctcccggggcccagcaccagctcagaataaagcgattccacagcaaaaaaaaaaaaaaaaaaaaaaaaaaaaaa

SEQ ID NO: 4 - DNA, 1024 bp, Homo sapiens
agcaagatggatctactgtggatcctgccctccctgtggcttctcctgcttggggggcctgcctgcctgaagacccaggaacaccccagctgcccaggacccagggaactggaagccagcaaagttgtcctcctgcccagttgtcccggagctccaggaagtcctggggagaagggagccccaggtcctcaagggccacctggaccaccaggcaagatgggccccaagggtgagccaggagatccagtgaacctgctccggtgccaggaaggccccagaaactgccgggagctgttgagccagggcgccaccttgagcggctggtaccatctgtgcctacctgagggcagggccctcccagtcttttgtgacatggacaccgaggggggcggctggctggtgtttcagaggcgccaggatggttctgtggatttcttccgctcttggtcctcctacagagcaggttttgggaaccaagagtctgaattctggctgggaaatgagaatttgcaccagcttactctccagggtaactgggagctgcgggtagagctggaagactttaatggtaaccgtactttcgcccactatgcgaccttccgcctcctcggtgaggtagaccactaccagctggcactgggcaagttctcagagggcactgcaggggattccctgagcctccacagtgggaggccctttaccacctatgacgctgaccacgattcaagcaacagcaactgtgcagtgattgtccacggtgcctggtggtatgcatcctgttaccgatcaaatctcaatggtcgctatgcagtgtctgaggctgccgcccacaaatatggcattgactgggcctcaggccgtggtgtgggccacccctaccgcagggttcggatgatgcttcgatagggcactctggcagccagtgcccttatctctcctgtacagcttccggatcgtcagccaccttgcctttgccaaccacctctgcttgcctgtccacatttaaaaataaaatcattttagc

SEQ ID NO: 5 - DNA, 1024 bp, Homo sapiens
cctgcatctttttggaaggattctttttataaatcagaaagtgttcgaggttcaaaggtttgcctcggagcgtgtgaacattcctccgctcggttttcaactcgcctccaacctgcgccgcccggccagcatgtctccccgcccgtgaagcggggctgccgcctccctgccgctccggctgccactaacgacccgccctcgccgccacctggccctcctgatcgacgacacacgcacttgaaacttgttctcagggtgtgtggaatcaactttccggaagcaaccagcccaccagaggaggtcccgagcgcgagcggagacgatgcagcggagactggttcagcagtggagcgtcgcggtgttcctgctgagctacgcggtgccctcctgcgggcgctcggtggagggtctcagccgccgcctcaaaagagctgtgtctgaacatcagctcctccatgacaaggggaagtccatccaagatttacggcgacgattcttccttcaccatctgatcgcagaaatccacacagctgaaatcagagctacctcggaggtgtcccctaactccaagccctctcccaacacaaagaaccaccccgtccgatttgggtctgatgatgagggcagatacctaactcaggaaactaacaaggtggagacgtacaaagagcagccgctcaagacacctgggaagaaaaagaaaggcaagcccgggaaacgcaaggagcaggaaaagaaaaaacggcgaactcgctctgcctggttagactctggagtgactgggagtgggctagaaggggaccacctgtctgacacctccacaacgtcgctggagctcgattcacggtaacaggcttctctggcccgtagcctcagcggggtgctctcagctgggttttggagcctcccttctgccttggcttggacaaacctagaattttctccctttatgtatctctatcgattgtgtagcaattgacagagaataactcagaatattgtctgccttaaagcagtacccccc

SEQ ID NO: 6 - DNA, 1024 bp, Homo sapiens
cacggccggagagacgcggaggaggagacatgagccggcgggcgcccagacggagcggccgtgacgctttcgcgctgcagccgcgcgccccgaccccggagcgctgacccctggccccacgcagctccgcgcccgggccggagagcgcaactcggcttccagacccgccgcgcatgctgtccccggactgagccgggcagccagcctcccacggacgcccggacggccggccggccagcagtgagcgagcttccccgcaccggccaggcgcctcctgcacagcggctgccgccccgcagcccctgcgccagcccggagggcgcagcgctcgggaggagccgcgcggggcgctgatgccgcagggcgcgccgcggagcgccccggagcagcagagtctgcagcagcagcagccggcgaggagggagcagcagcagcggcggcggcggcggcggcggcggcggaggcgcccggtcccggccgcgcggagcggacatgtgcaggctgggctaggagccgccgcctccctcccgcccagcgatgtattcagcgccctccgcctgcacttgcctgtgtttacacttcctgctgctgtgcttccaggtacaggtgctggttgccgaggagaacgtggacttccgcatccacgtggagaaccagacgcgggctcgggacgatgtgagccgtaagcagctgcggctgtaccagctctacagccggaccagtgggaaacacatccaggtcctgggccgcaggatcagtgcccgcggcgaggatggggacaagtatgcccagctcctagtggagacagacaccttcggtagtcaagtccggatcaagggcaaggagacggaattctacctgtgcatgaaccgcaaaggcaagctcgtggggaagcccgatggcaccagcaaggagtgtgtgttcatcgagaaggttctggagaacaactacacggccctgatgtcggctaagtactccggctggtacgtgggcttcaccaagaaggggcggccgcggaagggcc

SEQ ID NO: 7 - DNA, 738 bp, Homo sapiens
agtctcagattggtaaacacccgaactggtcaactctcaagagaccatctggttcaggttcctgactgggccagcgagtgaggatggccaggcctctggagcaggcggtagctgccatcgtgtgcaccttccaggaatacgcagggcgctgtggggacaaatacaagctctgccaggcggagctcaaggagctgctgcagaaggagctggccacctggaccccgactgagtttcgggaatgtgactacaacaaattcatgagtgttctggacaccaacaaggactgcgaggtggactttgtggagtatgtgcgctcacttgcctgcctctgtctctactgccacgagtacttcaaggactgcccctcagagcccccctgctcccagtagcctctgctccagggggtgcgctggctgtcgggggctgggcatgtctcccacaccccctcctaccctctctcctgtacccctttcaatctggacttgcccaggtcttctgcgatcagttaacccattttacctaggaggcccagagatgtgagggctccttcctcaggatgcccagcgaatgaggggtagagccactctggggcccagcctgcctgccgcacccctgtggcctcccttgtggatgggaggaggcgggatctgctctgaggccctcgaggctcagcagagcgtgcaccaatgagaccacgatgggaaagggcctatttaactcctaataaaaaactggcat

SEQ ID NO: 8 - DNA, 1024 bp, Homo sapiens
caactatggttgctaacacaagaatcggcactggtgacttttgagccatcaatcatcctttcctaacacatgaatcttttggaaaatggtctcaaatagtcaagaccccctgctgggaggagcttctatcctgctctgtggaagaaatgggcatgccagatgcctggaagatcagaggctcacattccagttcaaagttagagaaacctgagaggggtgtactggtatgaccacccgagatcccatcgctgagaaagataatggccctggagacaaagactgatgaagaaagacagtatccagctgtgatgcagatgaaacatgcggcatggttgcactcactatgcagccaaggctggaagccactctggatccagccactctggcacagcgttcttgctctaggaagaacaactgaacatggccacacaggcctcacagctggcatatggaggctgttaataagctgagcttgaggcttcaaaggactcttgtagcctgtgctggatctagcagtgaccagtatgccagtggccatggatgcccaggcccatgttcagtagtgaagcaggtgctagcagcagaagcaatagaaatggaaactgggagatctgctgtggatgaatctcattatcttctttctctgaccctctttggactaaataaactcccagtattcttgaccaacagaggtagggttggaacagaacctcaaaagtcatcattagacttcctactaatagggcattggaagttcaagcgactaactaggaaacaggttagtatttgtacccctacactgtgcaacagattacataccatttgctctatttggcatcctttccttaggctagattcccagaagaaagattactgattcgaagggcatgaacatttttatgactcttggcctaaattactaggccaaggttttttcaaagggatgcactaattttcactatttccccacaccctttcaggtgataaataattgacttgatttgtttacactgacgccaagaat

SEQ ID NO: 9 - DNA, 1024 bp, Homo sapiens
agtggttcactgcactgtgaaaacagattccagacgccgggaactcacgcctccaatcccagacgctatgtccagcaaaggctccgtggttctggcctacagtggcggcctggacacctcgtgcatcctcgtgtggctgaaggaacaaggctatgacgtcattgcctatctggccaacattggccagaaggaagacttcgaggaagccaggaagaaggcactgaagcttggggccaaaaaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccatgctcatttagacatcgaggccttcaccatggaccgggaagtgcgcaaaatcaaacaaggcctgggcttgaaatttgctgag

SEQ ID NO: 10 - DNA, 642 bp, Homo sapiens
atggtggccattgatcaggtccttgattggtgtcatcgaagtggcaagggtctttctgaagtatttgaacacactgttatatgtcactgtggagctgtgcattatgtgtgcagctgttcgccacctgatgaatcaagttcaaaaaaaggggtccccgtgaaagtgaccatcaagatggcaccacctcaccagacctccttggagctcttcgtgtacctgaacgaagtcacgggcaagcacggcatgggctgtattgacattatggagaactgcttcattagaataaagtccagaggtatctacaagaccccagcaggcaccatccttcaacaggctgatttagacatcgaggacttcactatggatcgggaagtgcacaaaatcaaccctgagtgtaaatctgtccaccattgcatcaccaagtcccaggagcaaatggaaggaaaagtgcaggtgcccatcttcaagggccaggtgcacatcctcagccaggagtctctactctctctctacaaggaggagctggtgagcgtcaacgtgcaggggaattatgagccaatcgatgccaccggtttcatcaacatcaattacctcagactgaaggaatatcattatttccagagtaaggtcactgtcaaatag

SEQ ID NO: 11 - DNA, 1024 bp, Homo sapiens
gcggtcctcgtcgcgaccggcccgggccgcaggacgccgcgctcgggggagctgagccacctctccgccaggcccgtgcgagcggtacaaagaaaagagcacttccctgctggggcagaggccaagccagagccctggccccatcacctctctgccctgcttgagtctggaatggacctccctgcctggaagctgcattgcccttggtcctgggcctgcctgccccctggggaacacaggcagcagccacattggcagtgaagccgtggcagcgtcggcagcagaggatgccccaggccctggagcgtgcagatggcagctgggcctgggtggtgctgctggccaccatggtgacccagggcctcaccctgggcttccccacgtgtatcggcatcttcttcactgaactgcaatgggagttccaggccagcaacagcgagacctcttggttcccctccatcctcacggctgtgctccacatggcagggcccctgtgcagcatcctggtgggacgcttcggctgccgagtgaccgtgatgctggggggcgtgctggccagcctgggcatggtggccagctccttctctcacaacctcagccagctctacttcacagcaggattcatcacaggcctgggcatgtgcttcagcttccagtcaagcatcacggtgctgggcttctactttgtccgccggcgggtgctggccaacgcgctggcctcgatgggcgtctccctgggcatcaccctctggccgctgctctcccgctaccttctggagaacctgggctggaggggtaccttccttgtcttcggcgggatctttctccactgctgcatctgcggggccatcataaggcctgtggccaccagtgtggcccctgagaccaaagaatgtcccccgccacctcccgagacacctgcacttggctgcctggctgcatgcggccggaccatccagcgccacctggccttcgacatcctgcggcacaacacaggctactgcgtgtacatactgggtgtgat

SEQ ID NO: 12 - DNA, 918 bp, Homo sapiens
ggggggagaggtagagatgagacaagaggtagaggggagaggtagaggtagccacgagctgataattacagacaagagatgcggagtatgtgggggctcattatcctgcatagtctatctttgtatatctttgaacttttcaagaataaaaaagcttaaaaagtatacatggcctggtcctaccagagactcacccaatgccagcctccagccagggagagccaagtttgcattttcacacgcatctcacactcctctgcactctcaacttggagagctccaaacagggaaaccccaagccttgctggcttctgccaaccccctgagcagaagcatgggtccccctgatcaccacctcaccaccctcatcctgatctcactgtacactccctttcctggtctgctaagtagcgggtgttttccttgacactaacgctacagctagaccacggtgggcttggcaacaggtgtcttcccagatgctggcgttaccgctagaccaaggagccctctggtggccccgtccgggcataacagaaagctcgcactcttgtcttctggtcactcctcactgtccccttcagcctcctatctctgtatggcctggtgtttcctaggttatgattgtagagcgaggattattataatattggaataaagaataattactacaaactaatgattaatgattcatatataatcatatctaagatctatatctagtataactattcttattttatatattttattatactggaacagctcgtgccctcggtctcttgcctcggcacctgggtggcttgctgcccacatccaccaagtgcactttgggaggctgaggctggaggactgctggaggccaggagttcaataccagcctgggcaacatagggagaccccccccccaccatctcc

SEQ ID NO: 13 - DNA, 1024 bp, Homo sapiens
ctccctttgggaagctgcccgccgagtctccgagatttgtccctggtggtcccgcggacccctcgtccctccgcagtctccggctggcagcgatggagggcgctggggagaacgccccggagtccagctcctctgcccctgggtccgaagagtctgccagggatccacaggtgccgcctccggaggaagaatcgggggactgcgcccggtccctggaggcggtccccaagaaactctgtgggtatttaagtaagttcggcggcaaagggcccatccggggctggaaatcccgctggttcttctacgacgaaaggaaatgtcagctgtattactcgcggaccgctcaggatgccaatcccttggacagcatcgacctctccagtgcagtgtttgactgtaaggcggacgctgaggaggggatcttcgaaatcaagactcccagccgggttattaccctgaaggccgccaccaagcaagcgatgctgtactggctgcagcagctgcagatgaagcgctgggaattccacaacagcccgccggcacctcctgccacccctgatgccgccctggctgggaatgggcccgtcctgcacctcgagctagggcaagaagaggcagagctggaggagttcctgtgccctgtgaaaacaccccctgggctagtgggcgtggcagctgccttgcagcccttccctgcccttcagaatatttccctcaagcacctggggactgaaatacagaacacaatgcacaacatccgtggcaacaagcaggcccagggaacaggccatgaacctccaggggaagattctccacagagtggggagcctcagagggaggagcagcccttggcctctgacgccagcaccccagggagagagccagaggattctccaaagcctgcacccaagccttctctgaccatcagtttcgctcagaaagccaagcgccagaacaacaccttcccattcttttctgaaggaatcacacggaaccgaactgcccaggagaaagtggcagcctt

SEQ ID NO: 14 - DNA, 1024 bp, Homo sapiens
ggaaagaagtggaataattaggaacctagggtggggtagggtagcaggacatttcaaacattaatgagcatatgagattccaggtcttgttaaaatgcaaattctgattcagctggtaggtgaggtctgagattgtgcatttctaacaagcactcagataatcttaaggctgttggccccagggtcacacttatagtgattttctagaacccagttggggaagtgaatcttgggcaggagaaatacacacctcttgcattgagtttggagatctcatctgatataactttttaagaaagaaaaataattttccaaatatccaattgataagctttcccactaagtggctttcccactaagtggctgcgttatgaaaattgcttcactttgaaacttctggtcttggtaatatagaatttctgtgttctcacagtgcttgattgagaatatgatattgagattatggcataaaatatagtggctgtacaaaaaaaaatacattattaggatctctaacaattatgtaaaagtcattgcttcatgggtagagctcaaactttggtgtgagacctggttttattcttggcacttactctgagttgtcttaggcaaattaataccttaagcaaaaatattctcatgtacattttacatgagaattataaatgaagtacataaagtccagcagtcacaaatgttatctattattaccatcgtcctaagactgcaatcagctatagtgaaagtagtctcaaagattgtttcataaatcatcagattcacctaattttctaaagaatttaaataaggagatggaatgaatagattgcattttgtttccatgcacaggggaactgtgcatatttcttctgtgactcggaaatggtttaacttttaaaaatcccaaaatagctgaagttagcagacatgcaatttaccaaggatgattggaatttttatctttcctgtaataatactatacccaagcacactgctcatgaggaaaacatttttatgtgaatct

SEQ ID NO: 15 - DNA, 1024 bp, Homo sapiens
ctggccccaaggtccgatcgcccaggggaggagcagcaccgggaccccgcgtcggctgggcgccccacaagggaagccagtcttaatatgatggaaacatctctgaacttctaaaagaccaaggttggcgttttagctctattaattttacttcgtcttggccagaattcacaatgacaacagtgacagtgaccacagaaattcccccaagggataagatggaagataattctgccttgtatgagtctacgtccgctcacattattgaagaaaccgagtatgtgaaaaagattcgaactactctgcaaaagatcaggacccagatgtttaaagatgaaataagacatgacagtacaaatcacaaactagatgcaaagcactgtggaaaccttcaacagggctctgattctgaaatggatccttcttgttgcagtttggatttgcttatgaaaaagataaaaggaaaagacctacagctcttagaaatgaacaaagagaatgaagtattgaaaatcaagctgcaagcctccagagaagcaggagcagcagctctgagaaacgtggcccagagattatttgaaaactaccaaacgcaatctgaagaagtgagaaagaagcaggaggacagtaaacaattactccaggttaacaagcttgaaaaagaacagaaattgaaacaacatgttgaaaatctgaatcaagttgctgaaaaacttgaagaaaaacacagtcaaattacagaattggagaaccttgtacagagaatggaaaaggaaaagagaacactactagaaagaaaactgtctttggaaaacaagctactgcaactcaaatccagtgctacatatggaaaaagttgccaggatcttcagagggagatttccattctccaggagcagatctctcatctgcagtttgtgattcactcccaacatcagaacctgcgcagtgtcatccaggagatggaaggattaaaaaataatttaaaagaacaagacaaaagaattgaaaatctcagagaaaa

SEQ ID NO: 16 - DNA, 817 bp, Homo sapiens
gaaggaggcccagacagtgagggcaggagggagagaagagacgcagaaggagagcgagcgagagagaaagggttctggattggaggggagagcaagggagggaggaaggcggtgagagaggcgggggcctcgggagggtgaaaggagggaggagaagggcggggcacggaggcccgagcgagggacaagactccgactccagctctgacttttttcgcggctctcggcttccactgcagccatgtcactcctcttgctggtggtctcagcccttcacatcctcattcttatactgcttttcgtggccactttggacaagtcctggtggactctccctgggaaagagtccctgaatctctggtacgactgcacgtggaacaacgacaccaaaacatgggcctgcagtaatgtcagcgagaatggctggctgaaggcggtgcaggtcctcatggtgctctccctcattctctgctgtctctccttcatcctgttcatgttccagctctacaccatgcgacgaggaggtctcttctatgccaccggcctctgccagctttgcaccagcgtggcggtgtttactggcgccttgatctatgccattcacgccgaggagatcctggagaagcacccgcgagggggcagcttcggatactgcttcgccctggcctgggtggccttccccctcgccctggtcagcggcatcatctacatccacctacggaagcgggagtgagcgccccgcctcgctcggctgcccccgccccttcccggcccccctcgccgcgcgtcctccaaaaaaataaaactttaacggcgg

SEQ ID NO: 17 - DNA, 1024 bp, Homo sapiens
gcggccgccgcagcgggcacggccaacgagctgcgggcccgggatcgcggcggctggacggggctggagctgtcgggagggcggagctacagggcctgcaaggatgttggctgtcccggagatgggcctgcaggggctgtacatcggctccagcccggagcggtccccagtgcctagcccacccggctccccgaggacccaggaaagctgcggcattgcccccctcacaccctcgcagtctccaaaacccgaggtccgagccccccagcaggcctccttctctgtggtggtggccattgacttcggcaccacgtctagtggctatgctttcagctttgccagtgaccctgaggccatccacatgatgaggaaatgggagggcggagacccgggcgtggcccaccagaagaccccgacctgcctgctgctgactccggagggcgccttccacagctttggctacaccgcccgcgattactaccatgacctggaccccgaagaggcgcgggactggctctacttcgagaagttcaagatgaagatccacagcgccacggatctcaccttgaagacccagctagaggcagtaaatggaaagacgatgcccgccctggaggtgttcgcccatgccctgcgcttcttcagggagcacgcccttcaggagctgagggagcagagcccatcgctgccagagaaggacactgtgcgctgggtgttgacggtgcctgccatctggaaacagccagccaagcagttcatgcgggaggctgcctacctggctggactagtgtcccgagagaatgcagagcagctactcatcgccctggagcccgaggccgcctcggtatactgccgcaagctgcgcctgcaccagctcctggacctgagtggccgggccccaggtggtgggcgcctgggtgagcgccgctccatcgactccagcttccgtcaggctcgggagcagctgcgaaggtcccgccacagccgcacgttcctggtggagtcaggcgtaggagagctgtgg

SEQ ID NO: 18 - DNA, 1024 bp, Homo sapiens
gccacctcggccgtggcaggaaccgaaacccggagcggccgaagctcagcgctccgctggggcagagggtcgcggcggctgggaacgccgctccccgacgcaccggccgcccccagcgcagcgcgctccgcggggtgctgatctgcctcttctccaagaaactcaaccactagtgacaatgaccagcctcctgactactccttctccaagagaagaactgatgaccaccccaattttacagcccactgaggccctgtccccagaagatggagccagcacagcactcattgcagttgttatcaccgttgtcttccttaccctgctctcggtcgtgatcttgatcttcttttacctgtacaagaacaaaggcagctacgtcacctatgaacctacagaaggtgagcccagtgccatcgtccagatggagagtgacttggccaagggcagcgagaaagaggaatatttcatctaatgactcccaggccccaaggagcttattcctggctccatcgctaacacgttgactgcttattatgggaaagttttctctgaagccagggagaagcattgattgatgtgggcaaatccaagctccagccaggtcgcagtcccaaatgccgacatcactgactccagggaccagggacatggagaaagctgtttatgatatctttaaccaggccctcttactagagctggtgtttgtgactggccaacaagatgtggctatgccaggggacatctgagtatgtgcccagtcatcttttttcacaggttgaagggagagaaaagattttgagttaaggtcattggctgctctactctgtcccctacctggtcacctagtgatagccccagtggagatactgtccatacaaggtcttcccagaggctggataccacagtaaaaggccaggccaggaggggtaggagactatggagatcttacctcctgataaatgtgctacaccccctaatctgagcccttcctttccgtgttccccaacaacctcatgcttacgtg

SEQ ID NO: 19 - DNA, 1024 bp, Homo sapiens
cagttacagggagcaccaccagggaacatctcggggagcctggttggaagctgcaggcttagtctgtcggctgcgggtctctgactgccctgtggggagggtcttgccttaacatcccttgcatttggctgcaaagaaatctgcttggaagaaggggttacgctgtttggccgggcagaaactccgctgagcagaacttgccgccagaatgctcctcctgttgctgagtatcatcgtcctccacgtcgcggtgctggtgctgctgttcgtctccacgatcgtcagccaatggatcgtgggcaatggacacgcaactgatctctggcagaactgtagcacctcttcctcaggaaatgtccaccactgtttctcatcatcaccaaacgaatggctgcagtctgtccaggccaccatgatcctgtcgatcatcttcagcattctgtctctgttcctgttcttctgccaactcttcaccctcaccaaggggggcaggttttacatcactggaatcttccaaattcttgctggtctgtgcgtgatgagtgctgcggccatctacacggtgaggcacccggagtggcatctcaactcggattactcctacggtttcgcctacatcctggcctgggtggccttccccctggcccttctcagcggtgtcatctatgtgatcttgcggaaacgcgaatgaggcgcccagacggtctgtctgaggctctgagcgtacatagggaagggaggaagggaaaacagaaagcagacaaagaaaaaagagctagcccaaaatcccaaactcaaaccaaaccaaacagaaagcagtggaggtgggggttgctgttgattgaagatgtatataatatctccggtttataaaacctatttataacactttttacatatatgtacatagtattgtttgctttttatgttgaccatcagcctcgtgttgagccttaaagaagtagctaaggaactttacatcctaacagtataatccagctcagtatttttgttttgttttttg

SEQ ID NO: 20 - DNA, 1024 bp, Homo sapiens
gcgcggaggcagggaaccggagccttggagcgacccaacccctcgtctcgctgccctcccgcgcctgcaacggtgcgcggagactccggcgaactcagacacccaacggcggagaacagaagcggcaggcggcggacgtggccgcgaagctgcgcgccgaacgcagcgcacccgctgccgagcagaggagccgcgcctttccccgaccctcggctccagcccccggcgcctgccgcctcgcagcccctctgcgtcctcggctcgggggccggcaccggcgatgccgagcggagcctccagtcctccgacccgctgaagaagcagcagccgctcgcccggagcctacggggattgtgcgagcggatcgtgctcggtggaggctcgggctgcggggcgcggggactccggggggcggggggagggaccgctctgtcggtgcccggcgccagccgcggctttgaagggtctccctcccctgcccttagcagctctgccacggactccgggaggctgcgggcggcgtcctgagggctccccagcagacccaatcggacttgagaaggtgatcgctctgctctcccaacccccttccctccccattccccccacttaactttttgtctccgttcatccgcggcttcgtcccctccccgcagacccacccgcggctgtgacaaccgcccggggcatgggccccccaacacggctcctagaggccccgcggcctcgcaagatgtgagaggccctccccgggcagaatcggagcttcaggagaggagctaataccccgccccccgtccctcacatcaggcggggtggaggtgcgcgctgagcccccgcggtgctgagcgtcccggagcgcccaatcctgggctggaacgagtagctggccggaggcgcgccgcggagagccggctgtcatgccctattgatccccctctgccccccgccaagtatgtttgggctggaccaattcgagccccaggtcaacagcaggaacgctggccagggcgagaggaacttt

SEQ ID NO: 21 - DNA, 1024 bp, Homo sapiens
gcgccccagtcgacgctgagctcctctgctactcagagttgcaacctcagcctcgctatggctcccagcagcccccggcccgcgctgcccgcactcctggtcctgctcggggctctgttcccaggacctggcaatgcccagacatctgtgtccccctcaaaagtcatcctgccccggggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggagctgtttgagaacacctcggccccctaccagctccagacctttgtcctgccagcgactcccccacaacttgtcagcccccgggtcctagaggtggacacgcaggggaccgtggtctgttccctggacgggctgttcccagtctcggaggcccaggtccacctggcactgggggaccagaggttgaaccccacagtcacctatggcaacgactccttctcggccaaggcctcagtcagtgtgaccgcagaggacgagggcacccagcggctgacgtgtgcagtaatactggggaaccagagccaggagacactgcagacagtgaccatctacagctttccggcgcccaacgtgattctgacgaagccagaggtct

SEQ ID NO: 22 - DNA, 1024 bp, Homo sapiens
ggaaaacgacacctagaagtaggagtgagattcgctgaagttcccttctgaggaagacccacccctccgcctggagagccggggctggcggtgcctgaggaccccttcggcctggacagcccacgcgggcttggggggcctcgctctgccctcatggggcggccatcggttcccgaagcggcgagtgaaaattcaaatggccagtagggggcgcactcggaagtggccgccccgcatgaggcagttcagcggccccgagagtccggggagggaggtttattctccgcctgcacgagactgtgaaatccgcaaccatgagcaggagaggcggccctggtggggaagaggccaccaacatctggacggcaggtacccagagagtgagcagctccacgcgggactgtgcacggtggccgacacccgcagggacgcccgccggacgagcacgcggagggccctcgcctccacggatgcaccatgccggtgtgaggagcatctgttcttcccactctctgcagttaacaaacccaacccaaaccaccacaggtgctcctcctggggagtttcctgtctgacaaatgccaggctcacttcaaggagaatcacgcttctttctaaagatggattcaccatttaaaacagagctctgggagcctttcggcaaatcttgaaagctgcacggcgcagagacatggatgtgacttcccaagcccggggcgtgggcctggagatgtacccaggcaccgcgcagcctgcggcccccaacaccacctcccccgagctcaacctgtcccacccgctcctgggcaccgccctggccaatgggacaggtgagctctcggagcaccagcagtacgtgatcggcctgttcctctcgtgcctctacaccatcttcctcttccccatcggctttgtgggcaacatcctgatcctggtggtgaacatcagcttccgcgagaagatgaccatccccgacctgtacttcatcaacctggcggtggcggacctcatcctggtggccgac

SEQ ID NO: 23 - DNA, 999 bp, Homo sapiens
ggggactcggggggcggccccttcctcgggaggcgcccaggtggcacgtcggtgcctcagctcccggggtctctgggctccgcgctgcccacggggtgccgaggggcctctggagggggcgggttctctcgagccagaaagtccggagccgcccaggaattttggctcgaactgagtgcatttcctctcgggccggccgggtgcgatccgggaagggcggctgcgggcagacgcggcgctgcgctcggccaggccggcaccatgcggcccctgctctgcgcgctgaccggactggccctgctccgcgccgcgggctctttggccgctgccgaacccttcagccctccgcgaggagactcagctcagagcacagcgtgtgacagacacatggctgtgcaacgccgtctagatgtcatggaggagatggtagagaagaccgtggatcacctggggacagaggtgaaaggcctgctgggcctgctggaggagctggcctggaacctgcccccgggacccttcagccccgctcccgaccttctcggagatggcttctgagccctggagctggagcccagcagttggaggtggtgcacctgccaggcagcgcccacagaaccagccctgtcctctcgacttccttccttagcttcatgtgaaataaaagctattctggtctcctctgtgtctgctgacagagtaacccgtttaactacagcctcctctcactccacttccatgcctggaggaagcctgcaaccccctccaggctcagacctggggacacccccactcctgtcatttataggggcagatggagcaggggttgattcacacagatgggggccctttgagtggccttgcttctcaaaatgtggccataggtgaaaagcaaggggatcagggtctcaggacccacccagactgtttaatccaaatctgcattgcaatgagacccccagggattttatgtgtccattaaagtggtttgttgttgttttt

SEQ ID NO: 24 - DNA, 1024 bp, Homo sapiens
gctgggcgcgggcggcggcggcccggaggagaacgggcggagggcgcgggccgaccgggcgcaccgaccatggcctccaaatgccccaagtgcgacaagaccgtgtacttcgccgagaaggtgagctccctggggaaggactggcacaagttctgcctcaagtgcgagcgctgcagcaagacgctgacgcccgggggccacgccgagcatgacgggaagccgttctgccacaagccgtgctacgccaccctgttcggacccaaaggcgtgaacatcgggggcgcgggctcctacatctacgagaagcccctggcggaggggccgcaggtcaccggccccatcgaggtccccgcggcccgagcagaggagcggaaggcgagcggccccccgaaggggcccagcagagcctccagtgtcaccactttcaccggggagcccaacacgtgcccgcgctgcagcaagaaggtgtacttcgctgagaaggtgacgtctctgggcaaggattggcaccggccctgcctgcgctgcgagcgctgcgggaagacactgacccccggcgggcacgcggagcacgacggccagccctactgccacaagccctgctatggaatcctcttcggacccaagggagtgaacaccggtgcggtgggcagctacatctatgaccgggaccccgaaggcaaggtccagccctaggctacagcggctctcatgatgtgggctcacctgcgccccagaccctgcaggggcccccctgcttggctctgctgggagagtgctcagccgcccagtcctgcctgcaagcccagggcgagtattggaggaggggcagccacgggcagagcaccatgcccatccccgagtctctggtgtgtctgccccctctggcatcctctgggcgtcccatgatcccttctgtgtctgcgtgtccgaatccccgtgtgaccctgtcccagcattttcccgccgaccctgcgtgtccccgtggcgctgtccgctctccctctcctgctgcccacccacc

SEQ ID NO: 25 - DNA, 1024 bp, Homo sapiens
cccggggccgcggagccgggccggggcagcgccgtctccgcctcggggccgccgggggcgccctgctgagcgctacccacgtgcgtccgcgccacctcgcgggcgaccccgcggccaaggcccccggcggagcggctcccgggcgccccgaactagcccccaactttgggcgaagtttgcctgcgcctctccccgcccccacgcggcgcgccggggccgcggacggcagcggcccccggggatgcgccttcccggggtacccctggcgcgccctgcgctgctgctgctgctgccgctgctcgcgccgctgctgggaacgggtgcgccggccgagctgcgggtccgcgtgcggctgccggacggccaggtgaccgaggagagcctgcaggcggacagcgacgcggacagcatcagcctcgagctgcgcaagcccgacggcaccctcgtctccttcaccgccgacttcaagaaggatgtgaaggtcttccgggccctgatcctgggggagctggagaaggggcagagtcagttccaggccctctgctttgtcacccagctgcagcacaatgagatcatccccagtgaggccatggccaagctccggcagaaaaatccccgggcagtgcggcaggcggaggaggttcggggtctggagcatctgcacatggatgtcgctgtcaacttcagccagggggccctgctgagcccccatctccacaacgtgtgtgccgaggccgtggatgccatctacacccgccaggaggatgtccggttctggctggagcaaggtgtggacagttctgtgttcgaggctctgcccaaggcctcagagcaggcggagctgcctcgctgcaggcaggtgggggaccacgggaagccctgcgtctgccgctatggcctgagcctggcctggtacccctgcatgctcaagtactgccacagccgcgaccggcccacgccctacaagtgtggcatccgcagctgccagaagagctacagcttcgacttctacgtgccccagagg

SEQ ID NO: 26 - DNA, 1024 bp, Homo sapiens
cttctgtgctgttccttcttgcctctaacttgtaaacaagacgtactaggacgatgctaatggaaagtcacaaaccgctgggtttttgaaaggatccttgggacctcatgcacatttgtggaaactggatggagagatttggggaagcatggactctttagccagcttagttctctgtggagtcagcttgctcctttctggaactgtggaaggtgccatggacttgatcttgatcaattccctacctcttgtatctgatgctgaaacatctctcacctgcattgcctctgggtggcgcccccatgagcccatcaccataggaagggactttgaagccttaatgaaccagcaccaggatccgctggaagttactcaagatgtgaccagagaatgggctaaaaaagttgtttggaagagagaaaaggctagtaagatcaatggtgcttatttctgtgaagggcgagttcgaggagaggcaatcaggatacgaaccatgaagatgcgtcaacaagcttccttcctaccagctactttaactatgactgtggacaagggagataacgtgaacatatctttcaaaaaggtattgattaaagaagaagatgcagtgatttacaaaaatggttccttcatccattcagtgccccggcatgaagtacctgatattctagaagtacacctgcctcatgctcagccccaggatgctggagtgtactcggccaggtatataggaggaaacctcttcacctcggccttcaccaggctgatagtccggagatgtgaagcccagaagtggggacctgaatgcaaccatctctgtactgcttgtatgaacaatggtgtctgccatgaagatactggagaatgcatttgccctcctgggtttatgggaaggacgtgtgagaaggcttgtgaactgcacacgtttggcagaacttgtaaagaaaggtgcagtggacaagagggatgcaagtcttatgtgttctgtctccctgacccctatgggtgttcctgtgccacaggc

SEQ ID NO: 27 - DNA, 1024 bp, Homo sapiens
gaattcggcacgagccgggtgcccagcgccgccgccgcccccgagctcccccgcgcccctgcccgcgggcggccggtgggcagcgggcgccatggccgcgccggagccgctgcggccgcgcctgtgccgcttggtgcgcggagagcagggctacggcttccacctgcacggcgagaagggccgccgcgggcagttcatccggcgcgtggaacccggttcccccgccgaggccgccgcgctgcgcgctggggaccgcctggtcgaggtcaacggcgtcaacgtggagggcgagacgcaccaccaggtggtgcaaaggatcaaggctgtggaggggcagactcggctgctggtggtggaccaggagacagatgaggagctccgccggcggcagctgacctgtaccgaggagatggcccagcgagggctcccacccgcccacgacccctgggagccgaagccagactgggcacacaccggcagccacagctccgaagctggcaagaaggatgtcagtgggcccctgagggagctgcgccctcggctctgccacctgcgaaagggacctcagggctatgggttcaacctgcatagtgacaagtcccggcccggccagtacatccgctctgtggacccgggctcacctgccgcccgctctggcctccgcgcccaggaccggctcattgaggtgaacgggcagaatgtggagggactgcgccatgctgaggtggtggccagcatcaaggcacgggaggacgaggcccggctgctggtcgtggaccccgagacagatgaacacttcaagcggcttcgggtcacacccaccgaggagcacgtggaaggtcctctgccgtcacccgtcaccaatggaaccagccctgcccagctcaatggtggctctgcgtgctcgtcccgaagtgacctgcctggttccgacaaggacactgaggagagcggcctccacctgagccccacggcggccgaggccaaggagaaggctcgagccatgcgagtcaacaagcgcgcg

SEQ ID NO: 28 - DNA, 1024 bp, Homo sapiens
aacaaacctcgcctggctcccagctggtgctgaagctcgtcagttcaccatccgccctcggcttccgcggggcgctgggccgccagcctcggcaccgtcctttcctttctccctcgcgttaggcaggtgacagcagggacatgtctcgggagatgcaggatgtagacctcgctgaggtgaagcctttggtggagaaaggggagaccatcaccggcctcctgcaagagtttgatgtccaggagcaggacatcgagactttacatggctctgttcacgtcacgctgtgtgggactcccaagggaaaccggcctgtcatcctcacctaccatgacatcggcatgaaccacaaaacctgctacaaccccctcttcaactacgaggacatgcaggagatcacccagcactttgccgtctgccacgtggacgcccctggccagcaggacggcgcagcctccttccccgcagggtacatgtacccctccatggatcagctggctgaaatgcttcctggagtccttcaacagtttgggctgaaaagcattattggcatgggaacaggagcaggcgcctacatcctaactcgatttgctctaaacaaccctgagatggtggagggccttgtccttatcaacgtgaacccttgtgcggaaggctggatggactgggccgcctccaagatctcaggatggacccaagctctgccggacatggtggtgtcccacctttttgggaaggaagaaatgcagagtaacgtggaagtggtccacacctaccgccagcacattgtgaatgacatgaaccccggcaacctgcacctgttcatcaatgcctacaacagccggcgcgacctggagattgagcgaccaatgccgggaacccacacagtcaccctgcagtgccctgctctgttggtggttggggacagctcgcctgcagtggatgccgtggtggagtgcaactcaaaattggacccaacaaagaccactctcctcaagatggcggactgtggcggcctcccgcagat

SEQ ID NO: 29 - DNA, 1024 bp, Homo sapiens
cgacgagccatggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgaattcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacagatatgcacttatacccataccattagcagtaattacaacatgtattgtgctgtatatgaatggtattctgaaatgtgacagaaaaccagacagaaccaactccaattgattggtaacagaagatgaagacaacagcataactaaattattttaaaaactaaaaagccatctgatttctcatttgagtattacaatttttgaacaactgttggaaatgtaacttgaagcagctgctttaagaagaaatacccactaacaaagaacaagcattagttttggctgtcatcaacttattatatgactaggtgcttgctttttttgtcagtaaattgtttttactgatgatgtagatacttttgtaaataaatgta

SEQ ID NO: 30 - DNA, 1024 bp, Homo sapiens
cagtttcagggtctcgccggcagccccagccggcgggcgcggagcgggcagcggggcccggatcgcaggctcctctgggggccccggagttgggaagcggcgtccgggagcccccggaggaggatgggctgcagacacagcaggctgagcagctgcaaacccccgaaaaagaagcgccaagaaccagaaccagaacagccacccagaccagagccccatgaattaggtcccctcaatggggacacagctataactgtccagctctgtgcatcagaggaggctgagcggcaccagaaggatataaccagaattctccagcaacatgaggaggaaaagaagaaatgggcacaacaggtggagaaggaaagggagctagagcttcgagacagactggatgagcagcaaagggtcctggaaggaaagaatgaagaggccctgcaagtcctccgggcctcatatgaacaggagaaagaagcgcttacccactctttccgggaggccagttctacccagcaggagaccatagacagactgacctcacagctggaggctttccaggccaaaatgaagagggtggaggagtccattctgagccgaaactataagaaacatatccaggattatgggagccccagccagttctgggagcaggagctggagagcttacactttgtcatcgagatgaagaatgagcgtattcatgagctggacaggcggctgatcctcatggaaacagtgaaagagaaaaatctgatattggaggaaaaaattacgaccctgcaacaggaaaatgaggacctccatgtccgaagccgcaaccaggtggtcctgtcaaggcagctgtcagaagacctgcttctcacgcgtgaggccctggagaaggaggtgcagctgcggcgacagctccagcaggagaaggaggagctgttgtaccgggtccttggggccaatgcctcgcctgccttccctctggcccctgtcactcccactgaggtctctttcctcgccacatagggtgcagggc

SEQ ID NO: 31 - DNA, 1024 bp, Homo sapiens
acatcttttcctttccccagaagagatccctaacctattgttttattgacagccttgctgttagaggctctttcccagaagttggacgaagaggctcaggcgttgctgtttcttgtcttccaagtcaagtggttactctggtaatggattgcctctctccgagctttcaccctggtgagactgtccagatctagtctgtaaacccagcttagaagcactgttgtaaaaatgactgaagagcccatcaaggagatcctgggagccccaaaggctcacatggcagcgacgatggagaagagccccaagagtgaagttgtgatcaccacagtccctctggtcagtgagattcagttgatggctgctacagggggtaccgagctctcctgctaccgctgcatcatcccctttgctgtggttgtcttcatcgccggcatcgtggtcaccgcggtggcttacagcttcaattcccatgggtctattatctccatctttggcctggttgttctgtcatctggactttttttactagcctccagtgccttgtgctggaaagtgagacaaaggagcaagaaagccaagagacgggagagtcaaacagctctcgtggcaaatcagagaagcttgtctgcttgagactgaatacgaccaaatgggccattgggcctggaaaacgtgctctgactttgtcacccaattcacccagaaccatggtgggagagaacagacttggcgttggagcagactggaagaatgggggtgggagggtggaggggcttctcctttgtgaggaatgactcatgtcttctttaacgacaaacttaaccctaagggctacttctgagactgaaaaatcagctttctatttacatgaaacactttgggggtcatgggagtgcacagcattagacagtatttggttcaccctgtaaagtagccaagaaaagatgagaaaaatcaagataggcctggcacactagacatttgcctccaaaagaaataacctacagtcttaagatgtatcata

SEQ ID NO: 32 - DNA, 1024 bp, Homo sapiens
gggcgcggagccccagccgagcctagccctgcccggccccggaggacttgcaacactccgaggccaggaacgctccgtctggaacggcgcaggtcccagcagctggggttccccctcagcccgtgagcagccatgtccaaccccagcgccccaccaccatatgaagaccgcaaccccctgtacccaggccctccgccccctgggggctatgggcagccatctgtcctgccaggagggtatcctgcctaccctggctacccgcagcctggctacggtcaccctgctggctacccacagcccatgccccccacccacccgatgcccatgaactacggcccaggccatggctatgatggggaggagagagcggtgagtgatagcttcgggcctggagagtgggatgaccggaaagtgcgacacacttttatccgaaaggtttactccatcatctccgtgcagctgctcatcactgtggccatcattgctatcttcacctttgtggaacctgtcagcgcctttgtgaggagaaatgtggctgtctactacgtgtcctatgctgtcttcgttgtcacctacctgatccttgcctgctgccagggacccagacgccgtttcccatggaacatcattctgctgaccctttttacttttgccatgggcttcatgacgggcaccatttccagtatgtaccaaaccaaagccgtcatcattgcaatgatcatcactgcggtggtatccatttcagtcaccatcttctgctttcagaccaaggtggacttcacctcgtgcacaggcctcttctgtgtcctgggaattgtgctcctggtgactgggattgtcactagcattgtgctctacttccaatacgtttactggctccacatgctctatgctgctctgggggccatttgtttcaccctgttcctggcttacgacacacagctggtcctggggaaccggaagcacaccatcagccccgaggactacatcactggcgccctgcagatttacacagacatcatct

SEQ ID NO: 33 - DNA, 780 bp, Homo sapiens
ggatataggcgcccccacacccgggcccggctaagcgccgccgccgctcctcgcctccttgctgcacgatggcctcgctccgggtggagcgcgccggcggcccgcgtctccctaggacccgagtcgggcggccggcagccgtccgcctcctccttctgctgggcgctgtcctgaatccccacgaggccctggctcagcctcttcccaccacaggcacaccagggtcagaaggggggacggtgaagaactatgagacagctgtccaattttgctggaatcattataaggatcaaatggatcctatcgaaaaggattggtgcgactgggccatgattagcaggccttatagcaccctgcgagattgcctggagcactttgcagagttgtttgacctgggcttccccaatcccttggcagagaggatcatctttgagactcaccagatccactttgccaactgctccctggtgcagcccaccttctctgaccccccagaggatgtactcctggccatgatcatagcccccatctgcctcatccccttcctcatcactcttgtagtatggaggagtaaagacagtgaggcccaggcctagggggcacgagcttctcaacaaccatgttactccacttccccacccccaccaggcctccctcctcccctcctactcccttttctcactctcatccccaccacagatccctggattgctgggaatggaagccagggttgggcatggcacaagttctgtaatcttcaaaataaaactttttttttga

SEQ ID NO: 34 - DNA, 1024 bp, Homo sapiens
gtctgcggaagtggtgacccgtgggacgcggctgagacaggagactgaaaggaaccataatttgtgacatcagttgttttctttgataagcagctatttatgattctggaagattaaggcagataggaaaccccatctgagattttaataaatccctcaaacaataaaccacatcatggacatacagctggaccctgccagagatgacctgcctctcatggccaacaccagccacatacttgtgaagcactatgtactggatttggatgtggattttgaaagtcaagtcattgaggggaccatagtgcttttcctcgaggatggaaacagattcaagaaacagaatagctctattgaggaagcctgccaatcagaatcaaacaaagcctgcaaatttgggatgcctgaaccctgccatattcccgtgacaaatgcaaggaccttctcatctgaaatggaatataatgattttgcaatctgtagtaaaggtgaaaaagatacttctgataaagatggtaaccatgacaaccaggaacatgcttctgggatttctagctcaaagtactgctgtgacacagggaatcatgggagtgaggattttttgctagtgttggactgctgtgatttatctgtgttaaaagtcgaggaggtggatgttgctgctgtgccaggtctggaaaaatttacaaggtctcctgagctcacggttgtttctgaggagttcaggaatcagattgtacgtgaacttgtgactttgcctgcaaatcgttggagggagcagttagactattacgctcgctgcagccaggctcctggctgtggggaactcctctttgacactgacacttggagcttgcagataaggaagacaggggctcagacagctactgactttcctcatgctatcaggatatggtacaaaactaaacctgaagggcgatcggttacatggacctcagaccagagtggcaggccatgtgtttatactgtgggatctcccataaacaacagggccctttttcca

SEQ ID NO: 35 - DNA, 1024 bp, Homo sapiens
cgccccccacgccctgggccccggagggccgcagccatgagtgaaatgtccagctttcttcacatcggggacatcgtctccctgtacgccgagggctccgtcaatggcttcatcagcactttggggctggtggatgaccgctgtgtggtggagcccgcggccggggacctggacaacccccctaagaagttccgtgactgcctcttcaaggtgtgccccatgaaccgctactcggcccagaagcagtactggaaggccaagcagactaagcaggacaaggagaagatcgctgatgtggtgttgctgcagaagctgcagcatgcggcgcagatggagcagaagcaaaatgacacggagaacaagaaggtgcatggggatgtcgtgaagtatggcagtgtgatccagctcctgcacatgaagagcaacaagtacctgacagtgaacaagcggcttccggccttgctggagaagaacgccatgcgggtgactctggatgccacaggcaacgagggttcctggctcttcatccagcccttctggaagctgcggagcaacggggacaacgtggtcgtgggggacaaggtgatcctgaatcctgtcaatgccgggcagcctctgcatgccagcaattacgagctcagcgacaacgccggctgcaaggaggtcaattctgtgaactgcaacaccagctggaagatcaacctgtttatgcagtttcgggaccacctggaggaggtgttgaaagggggagacgtggtgcggctgttccatgcggagcaggagaagttcctgacgtgtgacgagtacaagggcaagctgcaggtgttcctgcgaactacactgcgccagtctgccacctcggccaccagctccaatgctctctgggaggtggaggtggtccaccacgacccctgccgtggaggagctgggcactggaatggcttgtaccgcttcaagcacctggctacaggcaactacctggctgctgaggagaaccccagttacaaaggtgatgcctcagatc

SEQ ID NO: 36 - DNA, 1024 bp, Homo sapiens
gagatcatggcccgcctcttcagcccccggccgccccccagcgaagacctcttctacgagacctactacagcctgagccagcagtacccgctgctgctgctgctgctggggatcgtgctctgtgcgctcgcggcgctgctcgcagtggcctgggccagcggcagggagctgacctcagacccgagcttcctgaccactgtgctgtgcgcgctgggcggcttctcgctgctgctgggcctcgcttcccgggagcagcgactgcagcgctggacgcgtcccctgtccggcttggtatgggtcgcgctgctagcgctaggccacgccttcctgttcaccgggggcgtggtgagcgcctgggaccaggtgtcctattttctcttcgtcatcttcacggcgtatgccatgctgcccttgggcatgcgggacgccgccgtcgcgggcctcgcctcctcactctcgcatctgctggtcctcgggctgtatcttgggccacagccggactcacggcctgcactgctgccgcagttggcagcaaacgcagtgctgttcctgtgcgggaacgtggcaggagtgtaccacaaggcgctgatggagcgcgccctgcgggccacgttccgggaggcactcagctccctgcactcacgccggcggctggacaccgagaagaagcaccaggaacaccttctcttgtccatccttcctgcctacctggcccgagagatgaaggcagagatcatggcacggctgcaggcaggacaggggtcacggccagagagcactaacaatttccacagcctctatgtcaagaggcaccagggagtcagcgtgctgtatgctgacatcgtgggcttcacgcggctggccagcgagtgttcccctaaggagctggtgctcatgctcaatgagctctttggcaagttcgaccagattgccaaggagcatgaatgcatgcggatcaagatcctgggggactgttactactgtgtctctgggctgccactctcactgccagaccatgccatca

SEQ ID NO: 37 - DNA, 1024 bp, Homo sapiens
aaagttgctagctgggggcagagacggccacgcggttgcaggagtaagagatccctctgtggcgaattggccaacggctcctttcaaccctgcctcgttggtggccactggagaagcgcggggggctcccccagacagccgtggggacaagttagagccagcactttaccccgggccttgcgtgtagcttcccctcccctactctcggtgccctggtgtctggaggggggttgtgggggtgtgcccgccttacatggtccaccacccgggcaaccctctgggcttgtgttccatctcactcttgcttcctgtactgtggtcaaggggaaccactgcatcatgtcccggtatagctaccagagtctcctggactggctctatgggggcgtggaccccagttttgcaggcaatgggggccccgactgtgctgccttcctctcttggcagcagcggctgctggaaagtgtggtggtcctgaccctggctctgttggagatcctggtggccctgcggcacatcctgaggcagacgaaggaggacggtaggggtagccctggcagccagccagagcaggtgacccagcggccagaggaaggcaaggagagcctgagcaagaatctgctcttagtagccctgtgcctgaccttcggggtggaggtgggctttaagttcgccaccaagaccgtcatctacctgctcaacccctgtcacctggtcaccatgatgcatctcccctttgaattggagatttactacattcagcatgttatgctctacgtggtacccatctacctgctttggaaaggaggtgcttacactccagagcccctcagcagtttccggtgggctcttctctcaactggcctcatgttcttttatcacttcagcgtcttgcagatcctcggcctggtcaccgaagtgaatttgaacaacatgctgtgtccggccatctcagacccattctacggcccctggtatcgcatctgggcctcgggacaccagactctcatgaccatgacccacg

SEQ ID NO: 38 - DNA, 1024 bp, Homo sapiens
cagccggagcaggagcgccgagacgcgcctccggaacgtagagtaacaatcacaaccccacattccgggcgagcgtgagcacgagcgggaagggatgcgacccgggccgaggcgccgcgcgagcgccctgcagccaacgtgagcgccgccagccgcggcggcccgggcgccggccaggcctgggggcggcgggagcctgcgtgcgtgcactcctctcctctgctctcgtgcgtcctggaagcagtggccgcggcggctccctccggggtgcaaacccagtcgccgccagcagaacggccgacgctgcggaggggagaaggtcctttctcggctgccaccccctcccccggtcctccggggaagcagcggcttcagcaagattggacccgggcaccgggtggcactgaaccctctggccctcgccccagggggcccgtcggggagaggacgcagctcgtaggggggtccccggggagaggaagagacagcccctttcgagcttccacgcaccagccactccggggagggggccaagaggcaacggcggccaccaccgggcaccctcgccccctcccctcgggccgggagcttccagcccaagtctgcagcaccaggaagaaggcgcctgagctcccctcgcgacgagtcaaccgcagtaggaggtgggggcgaagagagggctgaacccgtccgctgcccgggcggtggagcccccacggcgaggcgctgcgccggcggtggagactcgcgttccctccagcccctggggcagaactttctcgccccccctcctccctcccccgcagtcggactccctccccagccggccagtcctcccggaggagaaggcgccgcggagacagcccgggcgggggcctaccttccccagggcaggcatcatgtcggcggcgcaggtgtcctcgtcccggagacaatcttgctacctgtgcgacctgccccgcatgccctgggccatgatctgggacttctcggaacccgtatgccgcggttgcgtcaactacga

SEQ ID NO: 39 - DNA, 1024 bp, Homo sapiens
agacgccgcccaggacgcagccgccgccgccgccgctcctctgccactggctctgcgccccagcccggctctgctgcagcggcagggaggaagagccgccgcagcgcgactcgggagccccgggccacagcctggcctccggagccacccacaggcctccccgggcggcgcccacgctcctaccgcccggacgcgcggatcctccgccggcaccgcagccacctgctcccggcccagaggcgacgacacgatgcgctgcgcgctggcgctctcggcgctgctgctactgttgtcaacgccgccgctgctgccgtcgtcgccgtcgccgtcgccgtcgccctcccagaatgcaacccagactactacggactcatctaacaaaacagcaccgactccagcatccagtgtcaccatcatggctacagatacagcccagcagagcacagtccccacttccaaggccaacgaaatcttggcctcggtcaaggcgaccacccttggtgtatccagtgactcaccggggactacaaccctggctcagcaagtctcaggcccagtcaacactaccgtggctagaggaggcggctcaggcaaccctactaccaccatcgagagccccaagagcacaaaaagtgcagacaccactacagttgcaacctccacagccacagctaaacctaacaccacaagcagccagaatggagcagaagatacaacaaactctggggggaaaagcagccacagtgtgaccacagacctcacatccactaaggcagaacatctgacgacccctcaccctacaagtccacttagcccccgacaacccacttcgacgcatcctgtggccaccccaacaagctcgggacatgaccatcttatgaaaatttcaagcagttcaagcactgtggctatccctggctacaccttcacaagcccggggatgaccaccaccctaccgtcatcggttatctcgcaaagaactcaacagacctccagtcagatgccagccagctctacggcccct

SEQ ID NO: 40 - DNA, 1024 bp, Homo sapiens
gtcactcgtataaaaacctatgctttgaaggttctcgtgtgtctcggcctgcaggtctcgctcagagctgtgtccctgaacatccaccctgctggggtggcttgacgcacttctgtgcaaatctgttcgctcgcaaccctacctacctctctcccgaaccggagaaaaccttcggcggggtccttccgggttttgtgtcgaatctgcggcggcgacccggcgccgcgtcacgcggtggtgaatgtgcggcagtacgcgcgccgcgtcgtttacgcggcgatttcatcatgctccgagccggcggcgcgcgccgcttccgtcgccaccctctctggacagcccagggccgcaggctcatgccctctccgcgtccagtgctgcttagaggtgctcgcgccgctctgctgctgctgctgccgccccggctcttagcccgaccctcgctcctcctccgccggtccctcagcgcggcctcctgcgccccgatctccttgcccgccgccgcctcccggagcagcatggacggcgcgggggctgaggaggtgctggcacctctgaggctagcagtgcgccagcagggagatcttgtgcgaaaactcaaagaagataaagcaccccaagtagacgtagacaaagcagtggctgagctcaaagcccgcaagagggttctggaagcaaaggagctggcgttacagcccaaagatgatattgtagaccgagcaaaaatggaagataccctgaagaggaggtttttctatgatcaagcttttgctatttatggaggtgttagtggtctgtatgactttgggccagttggctgtgctttgaagaacaatattattcagacctggaggcagcactttatccaagaggaacagatcctggagatcgattgcaccatgctcacccctgagccagttttaaagacctctggccatgtagacaaatttgctgacttcatggtgaaagacgtaaaaaatggagaatgttttcgtgctgaccatctattaaaagctcatttaca

SEQ ID NO: 41 - DNA, 1024 bp, Homo sapiens
gtctcctctggatcttaactactgagcgcaatgctgagccatggagccgggttggccttgtggatcacactgagcctgctgcagactggactggcggagccagagagatgtaacttcaccctggcggagtccaaggcctccagccattctgtgtctatccagtggagaattttgggctcaccctgtaactttagcctcatctatagcagtgacaccctgggggccgcgttgtgccctacctttcggatagacaacaccacatacggatgtaaccttcaagatttacaagcaggaaccatctataacttcaagattatttctctggatgaagagagaactgtggtcttgcaaacagatcctttacctcctgctaggtttggagtcagtaaagagaagacgacttcaaccggcttgcatgtttggtggactccttcttccggaaaagtcacctcatatgaggtgcaattatttgatgaaaataaccaaaagatacagggggttcaaattcaagaaagtacttcatggaatgaatacacttttttcaatctcactgctggtagtaaatacaatattgccatcacagctgtttctggaggaaaacgttctttttcagtttataccaatggatcaacagtgccatctccagtgaaagatattggtatttccacaaaagccaattctctcctgatttcctggtcccatggttctgggaatgtggaacgataccggctgatgctaatggataaagggatcctagttcatggcggtgttgtggacaaacatgctacttcctatgcttttcacgggctgtcccctggctacctctacaacctcactgttatgactgaggctgcagggctgcaaaactacaggtggaaactagtcaggacagcccccatggaagtctcaaatctgaaggtgacaaatgatggcagtttgacctctctaaaagtcaaatggcaaagacctcctggaaatgtggattcttacaatatcaccctgtctcacaaagggaccatcaagg

SEQ ID NO: 42 - DNA, 1024 bp, Homo sapiens
attccacgttcttaactgttccattttccgtatctgcttcgggcttccacctcatttttttcgctttgcccattctgtttcagccagtcgccaagaatcatgaaagtcgccagtggcagcaccgccaccgccgccgcgggccccagctgcgcgctgaaggccggcaagacagcgagcggtgcgggcgaggtggtgcgctgtctgtctgagcagagcgtggccatctcgcgctgcgccgggggcgccggggcgcgcctgcctgccctgctggacgagcagcaggtaaacgtgctgctctacgacatgaacggctgttactcacgcctcaaggagctggtgcccaccctgccccagaaccgcaaggtgagcaaggtggagattctccagcacgtcatcgactacatcagggaccttcagttggagctgaactcggaatccgaagttggaacccccgggggccgagggctgccggtccgggctccgctcagcaccctcaacggcgagatcagcgccctgacggccgaggtgagatccagatccgaccactagatcatccttataccgacggggaaacggaggccagagagggcgtgggcgcttgcaccacttccgtcccatccttgcgggtacctggctatgcgggggtgcctaaggagcctggaaaaagcgctcccccgtcgtgcttcctggggaagggggcgttcgctgcgctcggagcggcgtcccttccaacccgccggtctcatttcttctcgttttcacaggcggcatgcgttcctgcggacgatcgcatcttgtgtcgctgaagcgcctcccccagggaccggcggaccccagccatccagggggcaagaggaattacgtgctctgtgggtctcccccaacgcgcctcgccggatctgagggagaacaagaccgatcggcggccactgcgcccttaactgcatccagcctggggctgaggctgaggcactggcgaggagagggcgctcctctctgcacacctactagtcaccagagactt

SEQ ID NO: 43 - DNA, 1024 bp, Homo sapiens
gggccggggggcggagccttgcgggctggagcgaaagaatgcgggggctgagcgcagaagcggctcgaggctggaagaggatcttgggcgccgccaggttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagag

SEQ ID NO: 44 - DNA, 1024 bp, Homo sapiens
gaattttctgcaaggatcatatctaagtgcactttttgctgatacttcatttctagacattgagtctcactctaccccccaggctgaagtgcagtggtgtgatctcggttcactgcaacctccgcctccaggttcaagtgattctcgtacctcagcctcccgagtagctgggattacaggcgcctgccaccatgcctggctgatatttatatttttagtagagatggagtttcaccatgttggccaggctggtctcgaactctggacctcagatcttgtagaaaatttcagctgtagcccttggactagaagctgaaataacagaagctgtgtacgatgcattagggtattgaagaaaattaacttttgaattaaatatttggaatataaggaaataaggaaagttgactgaaaatggggcggaagaaaatacaaatcacacgcataatggatgaaaggaaccgacaggtcacttttacaaagagaaagtttggattaatgaagaaagcctatgaacttagtgtgctctgtgactgtgaaatagcactcatcattttcaacagctctaacaaactgtttcaatatgctagcactgatatggacaaagttcttctcaagtatacagaatataatgaacctcatgaaagcagaaccaactcggatattgttgaggctctgaacaagaaggaacacagagggtgcgacagcccagaccctgatacttcatatgtgctaactccacatacagaagaaaaatataaaaaaattaatgaggaatttgataatatgatgcggaatcataaaatcgcacctggtctgccacctcagaacttttcaatgtctgtcacagttccagtgaccagccccaatgctttgtcctacactaacccagggagttcactggtgtccccatctttggcagccagctcaacgttaacagattcaagcatgctctctccacctcaaaccacattacatagaaatgtgtctcctggagctcctcagagaccaccaagtactggca

SEQ ID NO: 45 - DNA, 1024 bp, Homo sapiens
agcactctccagcctctcaccgcaaaattacacaccccagtacaccagcagaggaaacttataacctcgggaggcgggtccttcccctcagtgcggtcacatacttccagaagagcggaccagggctgctgccagcacctgccactcagagcgcctctgtcgctgggacccttcagaactctctttgctcacaagttaccaaaaaaaaaagagccaacatgttggtattgctggctggtatctttgtggtccacatcgctactgttattatgctatttgttagcaccattgccaatgtctggttggtttccaatacggtagatgcatcagtaggtctttggaaaaactgtaccaacattagctgcagtgacagcctgtcatatgccagtgaagatgccctcaagacagtgcaggccttcatgattctctctatcatcttctgtgtcattgccctcctggtcttcgtgttccagctcttcaccatggagaagggaaaccggttcttcctctcaggggccaccacactggtgtgctggctgtgcattcttgtgggggtgtccatctacactagtcattatgcgaatcgtgatggaacgcagtatcaccacggctattcctacatcctgggctggatctgcttctgcttcagcttcatcatcggcgttctctatctggtcctgagaaagaaataaggccggacgagttcatggggatctggggggtggggaggaggaagccgttgaatctgggagggaagtggaggttgctgtacaggaaaaaccgagataggggaggggggagggggaagcaaaggggggaggtcaaatcccaaaccattactgaggggattctctactgccaagcccctgccctggggagaaagtagttggctagtactttgatgctcccttgatggggtccagagagcctccctgcagccaccagacttggcctccagctgttcttagtgacacacactgtctggggccccatcagctgccacaacaccagccccacttctggg

SEQ ID NO: 46 - DNA, 1024 bp, Homo sapiens
gggaagctcccctccgagactctggatagccgccgggggggcgttgcctggccggctggttgcgtagcaacggcaggcggtgcgtgacgtgcgcagccgcgttccgtcctgaggcgcgcccgccccggggtaagctcgcgccgccgcgtcagctcagcgctgggtctctcggtcccgcagccgtgaggaggacggtctgcatactcgctgcccgccggctccctcccccgcgtccctgcgaccgccgcggcgaagatggcctcaggagtgcaagtagctgatgaagtatgtcgcattttttatgacatgaaagttcgtaaatgctccacaccagaagaaatcaagaaaagaaagaaggctgtcattttttgtctcagtgcagacaaaaagtgcatcattgtagaagaaggcaaagagatcttggttggagatgttggtgtaaccataactgatcctttcaagcattttgtgggaatgcttcctgaaaaagattgtcgctatgctttgtatgatgcaagctttgaaacaaaagaatccagaaaagaagagttgatgttttttttgtgggcaccagaactagcacctctgaaaagtaaaatgatctatgcaagctccaaggatgcaattaaaaagaaatttcaaggcataaaacatgaatgtcaagcaaatggaccagaagatctcaatcgggcttgtattgctgaaaagttaggtggatccttaattgtagcctttgaaggatgccctgtgtagattattcagtgccacaaattgaaagcttccatgtttaatgttatcctcttgctatataaataaagcaaatatatttaggccagggtctcactgagggggagctgtcttgtcatcttttagagtaaactattctataaacatatgcaaacagccctaaataaatctaaagtctaaagttttattgatgtgaaattaaattcttattggccaaatgcctgttttgatgagttgatttataaagatttttgttaagctcaggattttaaattac

SEQ ID NO: 47 - DNA, 1024 bp, Homo sapiens
agacactgcccgctctccgggactccgcgccgctccccgttgccttccaggactgagaaaggggaaagggaagggtgccacgtccgagcagccgccttgactggggaagggtctgaatcccacccttggcattgcttggtggagactgagatacccgtgctccgctcgcctccttggttgaagatttctccttccctcacgtgatttgagccccgtttttattttctgtgagccacgtcctcctcgagcggggtcaatctggcaaaaggagtgatgcgcttcgcctggaccgtgctcctgctcgggcctttgcagctctgcgcgctagtgcactgcgcccctcccgccgccggccaacagcagcccccgcgcgagccgccggcggctccgggcgcctggcgccagcagatccaatgggagaacaacgggcaggtgttcagcttgctgagcctgggctcacagtaccagcctcagcgccgccgggacccgggcgccgccgtccctggtgcagccaacgcctccgcccagcagccccgcactccgatcctgctgatccgcgacaaccgcaccgccgcggcgcgaacgcggacggccggctcatctggagtcaccgctggccgccccaggcccaccgcccgtcactggttccaagctggctactcgacatctagagcccgcgaagctggcgcctcgcgcgcggagaaccagacagcgccgggagaagttcctgcgctcagtaacctgcggccgcccagccgcgtggacggcatggtgggcgacgacccttacaacccctacaagtactctgacgacaacccttattacaactactacgatacttatgaaaggcccagacctgggggcaggtaccggcccggatacggcactggctacttccagtacggtctcccagacctggtggccgacccctactacatccaggcgtccacgtacgtgcagaagatgtccatgtacaacctgagatgcgcggcggaggaaaactgtctggccagtacagcataca

SEQ ID NO: 48 - DNA, 1024 bp, Homo sapiens
ctgtgcgccctgcgcgccctgcgcacccgcggcccgagcccagccagagccgggcggagcggagcgcgccgagcctcgtcccgcggccgggccggggccgggccgtagcggcggcgcctggatgcggacccggccgcggggagacgggcgcccgccccgaaacgactttcagtccccgacgcgccccgcccaacccctacgatgaagagggcgtccgctggagggagccggctgctggcatgggtgctgtggctgcaggcctggcaggtggcagccccatgcccaggtgcctgcgtatgctacaatgagcccaaggtgacgacaagctgcccccagcagggcctgcaggctgtgcccgtgggcatccctgctgccagccagcgcatcttcctgcacggcaaccgcatctcgcatgtgccagctgccagcttccgtgcctgccgcaacctcaccatcctgtggctgcactcgaatgtgctggcccgaattgatgcggctgccttcactggcctggccctcctggagcagctggacctcagcgataatgcacagctccggtctgtggaccctgccacattccacggcctgggccgcctacacacgctgcacctggaccgctgcggcctgcaggagctgggcccggggctgttccgcggcctggctgccctgcagtacctctacctgcaggacaacgcgctgcaggcactgcctgatgacaccttccgcgacctgggcaacctcacacacctcttcctgcacggcaaccgcatctccagcgtgcccgagcgcgccttccgtgggctgcacagcctcgaccgtctcctactgcaccagaaccgcgtggcccatgtgcacccgcatgccttccgtgaccttggccgcctcatgacactctatctgtttgccaacaatctatcagcgctgcccactgaggccctggcccccctgcgtgccctgcagtacctgaggctcaacgacaacccctgggtgtgtgactgccgggcacgcccactctgggcctggc

SEQ ID NO: 49 - DNA, 381 bp, Homo sapiens
atgcatctctcccagctgctggcctgcgccctgctgctcacgctgctctccctccggccctccgaagccaagcccggggcgccgccgaaggtcccgcgaaccccgccggcagaggagctggccgagccgcaggctgcgggcggcggtcagaagaagggcgacaaggctcccgggggcgggggcgccaatctcaagggcgaccggtcgcgactgctccgggacctgcgcgtggacaccaagtcgcgggcagcgtgggctcgccttctgcaagagcaccccaacgcgcgcaaatacaaaggagccaacaagaagggcttgtccaagggctgcttcggcctcaagctggaccgaatcggctccatgagcggcctgggatgttag

SEQ ID NO: 50 - DNA, 1024 bp, Homo sapiens
gaaactaggggcaaggagacgaagagaacatgaaagttaaactttaagatgaagaacaaagctgaacatactgatgcattggatctttggagaggatctcagaactcattgtacttaatttacaggctaaaaccttagaagaggaatttattatatcctacacaagactccagggaagcacatggccttggactgaaggctggcatctggaagctgtcagccaccagcaccttctgcagcaggaaaaggccagggctctgctggagcaggcagcagagtggacgcacagtaacatgggcaacttgaagagcgtggcccaggagcctgggccaccctgcggcctggggctggggctgggccttgggctgtgcggcaagcagggcccagccaccccggcccctgagcccagccgggccccagcatccctactcccaccagcgccagaacacagccccccgagctccccgctaacccagcccccagaggggcccaagttccctcgtgtgaagaactgggaggtggggagcatcacctatgacaccctcagcgcccaggcgcagcaggatgggccctgcaccccaagacgctgcctgggctccctggtatttccacggaaactacagggccggccctcccccggccccccggcccctgagcagctgctgagtcaggcccgggacttcatcaaccagtactacagctccattaagaggagcggctcccaggcccacgaacagcggcttcaagaggtggaagccgaggtggcagccacaggcacctaccagcttagggagagcgagctggtgttcggggctaagcaggcctggcgcaacgctccccgctgcgtgggccggatccagtgggggaagctgcaggtgttcgatgcccgggactgcaggtctgcacaggaaatgttcacctacatctgcaaccacatcaagtatgccaccaaccggggcaaccttcgctcggccatcacagtgttcccgcagcgctgccctggccgaggagacttccgaatctg

SEQ ID NO: 51 - DNA, 837 bp, Homo sapiens
gctcctcctgcacacctccctcgctctcccacaccactggcaccaggccccggacacccgctctgctgcaggagaatggctactcatcacacgctgtggatgggactggccctgctgggggtgctgggcgacctgcaggcagcaccggaggcccaggtctccgtgcagcccaacttccagcaggacaagttcctggggcgctggttcagcgcgggcctcgcctccaactcgagctggctccgggagaagaaggcggcgttgtccatgtgcaagtctgtggtggcccctgccacggatggtggcctcaacctgacctccaccttcctcaggaaaaaccagtgtgagacccgaaccatgctgctgcagcccgcggggtccctcggctcctacagctaccggagtccccactggggcagcacctactccgtgtcagtggtggagaccgactacgaccagtacgcgctgctgtacagccagggcagcaagggccctggcgaggacttccgcatggccaccctctacagccgaacccagacccccagggctgagttaaaggagaaattcaccgccttctgcaaggcccagggcttcacagaggataccattgtcttcctgccccaaaccgataagtgcatgacggaacaataggactccccagggctgaagctgggatcccggccagccaggtgacccccacgctctggatgtctctgctctgttccttccccgagcccctgccccggctccccgccaaagcaaccctgcccactcaggcttcatcctgcacaataaactccggaagcaagtcagtaaaaaaaaaaaaaaaaaaaaaaaaaa

SEQ ID NO: 52 - DNA, 1024 bp, Homo sapiens
cggctgcggttcctggtgctgctcggcgcgcggccagctttcggaacggaacgctcggcgtcgcgggccccgcccggaaagtttgccgtggagtcgcgacctcttggcccgcgcggcccggcatgaagcggcgttgaggagctgctgccgccgcttgccgctgccgccgccgccgcctgaggaggagctgcagcaccctgggccacgccgatgactactgcaaactgtggcgcccacgacgagctcgacttcaaactcgtctttggcgaggacggggcgccggcgccgccgcccccgggctcgcggcctgcagatcttgagccagatgattgtgcatccatttacatctttaatgtagatccacctccatctactttaaccacaccactttgcttaccacatcatggattaccgtctcactcttctgttttgtcaccatcgtttcagctccaaagtcacaaaaactatgaaggaacttgtgagattcctgaatctaaatatagcccattaggtggtcccaaaccctttgagtgcccaagtattcaaattacatctatctctcctaactgtcatcaagaattagatgcacatgaagatgacctacagataaatgacccagaacgggaatttttggaaaggccttctagagatcatctctatcttcctcttgagccatcctaccgggagtcttctcttagtcctagtcctgccagcagcatctcttctaggagttggttctctgatgcatcttcttgtgaatcgctttcacatatttatgatgatgtggactcagagttgaatgaagctgcagcccgatttacccttggatcccctctgacttctcctggtggctctccagggggctgccctggagaagaaacttggcatcaacagtatggacttggacactcattatcacccaggcaatctccttgccactctcctagatccagtgtcactgatgagaattggctgagccccaggccagcctcaggaccctcatcaaggcccacatccccctgtg

SEQ ID NO: 53 - DNA, 1024 bp, Homo sapiens
gggagaaacgttctcactcgctctctgctcgctgcgggcgctccccgccctctgctgccagaaccttggggatgtgcctagacccggcgcagcacacgtccgggccaaccgcgagcagaacaaacctttggcgggcggccaggaggctccctcccagccaccgcccccctccagcgcctttttttccccccatacaatacaagatcttccttcctcagttcccttaaagcacagcccagggaaacctcctcacagttttcatccagccacgggccagcatgtctgggggcaaatacgtagactcggagggacatctctacaccgttcccatccgggaacagggcaacatctacaagcccaacaacaaggccatggcagacgagctgagcgagaagcaagtgtacgacgcgcacaccaaggagatcgacctggtcaaccgcgaccctaaacacctcaacgatgacgtggtcaagattgactttgaagatgtgattgcagaaccagaagggacacacagttttgacggcatttggaaggccagcttcaccaccttcactgtgacgaaatactggttttaccgcttgctgtctgccctctttggcatcccgatggcactcatctggggcatttacttcgccattctctctttcctgcacatctgggcagttgtaccatgcattaagagcttcctgattgagattcagtgcatcagccgtgtctattccatctacgtccacaccgtctgtgacccactctttgaagctgttgggaaaatattcagcaatgtccgcatcaacttgcagaaagaaatataaatgacatttcaaggatagaagtatacctgattttttttccttttaattttcctggtgccaatttcaagttccaagttgctaatacagcaacaatttatgaattgaattatcttggttgaaaataaaaagatcactttctcagttttcataagtattatgtctcttctgagctatttcatctatttttggcagtctgaatttttaaaacc

SEQ ID NO: 54 - DNA, 1024 bp, Homo sapiens
ctagtattctactagaactggaagattgctctccgagttttttttttgttattttgttaaaaaataaaaagcttgagcagcaattcatattactgtcacaggtatttttgctgtgctgtgcaaggtaactctgctagctaagattcacaatgttgaaagcccttttcctaactatgctgactctggcgctggtcaagtcacaggacaccgaagaaaccatcacgtacacgcaatgcactgacggatatgagtgggatcctgtgagacagcaatgcaaagatattgatgaatgtgacattgtcccagacgcttgtaaaggtggaatgaagtgtgtcaaccactatggaggatacctctgccttccgaaaacagcccagattattgtcaataatgaacagcctcagcaggaaacacaaccagcagaaggaacctcaggggcaaccaccggggttgtagctgccagcagcatggcaaccagtggagtgttgcccgggggtggttttgtggccagtgctgctgcagtcgcaggccctgaaatgcagactggccgaaataactttgtcatccggcggaacccagctgaccctcagcgcattccctccaacccttcccaccgtatccagtgtgcagcaggctacgagcaaagtgaacacaacgtgtgccaagacatagacgagtgcactgcagggacgcacaactgtagagcagaccaagtgtgcatcaatttacggggatcctttgcatgtcagtgccctcctggatatcagaagcgaggggagcagtgcgtagacatagatgaatgtaccatccctccatattgccaccaaagatgcgtgaatacaccaggctcattttattgccagtgcagtcctgggtttcaattggcagcaaacaactatacctgcgtagatataaatgaatgtgatgccagcaatcaatgtgctcagcagtgctacaacattcttggttcattcatctgtcagtgcaatcaaggatatgagctaagcagtgacaggctcaactg

SEQ ID NO: 55 - DNA, 1024 bp, Homo sapiens
gtggttcagaggcagcttctagacctgcaggagggagattgtattcagaggaagagcatcattttggcaacatctgaaagtgaaaacggaagccagaaacacttggccagccctgggggatttttttcttctatgcctctctggtggaatgacatttgctgtgtaggcatctttcctctgactgtatttcttggccttgaagagtactgagtttaaaaagacagtatgtgacagtccatggaaattgcctcttctgtgaaatctcgccacctgctccgaagacatgttgttgtctcccaaattctccttatccaccattcacatacgactgacggccaaaggattgcttcgaaaccttcgacttccttcagggtttaggagaagcactgttgttttccacacagttgaaaagagcaggcaaaagaatcctcgaagcttatgtatccagccacagacagctcccgatgcgctgccccctgagaaaacacttgaattgacgcaatataaaacaaaatgtgaaaaccaaagtggatttatcctgcagctcaagcagcttcttgcctgtggtaataccaagtttgaggcattgacagttgtgattcagcacctgctgtctgagcgggaggaagcactgaaacaacacaaaaccctatctcaagaacttgttaacctccggggagagctagtcactgcttcaaccacctgtgagaaattagaaaaagccaggaatgagttacaaacagtgtatgaagcattcgtccagcagcaccaggctgaaaaaacagaacgagagaatcggcttaaagagttttacaccagggagtatgaaaagcttcgggacacttacattgaagaagcagagaagtacaaaatgcaattgcaagagcagtttgacaacttaaatgctgcgcatgaaacctctaagttggaaattgaagctagccactcagagaaacttgaattgctaaagaaggcctatgaagcctccctttcagaaattaagaaaggccatgaaatagaaaag

SEQ ID NO: 56 - DNA, 423 bp, Homo sapiens
ccggaagtgactgcggacgaatcggcgtttgccgaggctggcatagatttggctgtctccgctcatagctgcttttggcgcgaaagatgccgggtctggttgactcaaaccctgccccgcctgagtctcaggagaagaagccgctgaagccctgctgcgcttgcccggagaccaagaaggcgcgcgatgcgtgtatcatcgagaaaggagaagaacactgtggacatctaattgaggcccacaaggaatgcatgagagccctaggatttaaaatatgaaatggtggtctgctgtgtgaataaataattcctgaagaatgaagaagattaattttgggagttctttgacgaactttgatatgtggaaaaagtatttataatttattgtaagaagaaagtaaaatattactagtggaagatcttc

SEQ ID NO: 57 - DNA, 1024 bp, Homo sapiens
tttcctctttctctaagagtctctctctccctttccctctctctccccccaatctgtctttctagcatgttgccctttttcaaccacatttgtgtttcaggtgtagagaggagagagagtgaacagggagcggggcttttgtctgttggtctccctggactgaagagagggagaatagaagcccaagactaagattctcaaaatggtttattacccagaactctttgtctgggtcagtcaagaaccatttccaaacaaggacatggagggaaggcttcctaagggaagacttcctgtcccaaaggaagtgaaccgcaagaagaacgatgagacaaacgctgcctccctgactccactgggcagcagtgaactccgctccccaagaatcagttacctccactttttttaatcgtaacacctccatttgtattacatatggtgtatgggtattgatgaggtcatggtatcatatatgggatttttttctgtgtaaatcatcaagtataagaagaaactatgggactctgagccttgctttagagaatttacagtggacaaataggtgtcatcaaaccagtttttaatcattctgactcaagtgaaaacgctcagaatttcacactgtgaatccacgtttacaacccttacaggtgggccttcaggcctggttcgctacaacaatgtcttccacaactcaaactcccaccgcgctcacacaaccggtccactcctgccttttcactcacacagctcccgactgcttcttgcagaggctgagagtccccccccccacctttttttttcatttagatgtaacaaacctagtagtttatgttcatcaattgtctgtatatctctatattttatccatgtactcttttgatgtatagaagtagtttgaaactcattgtttccttgtggtaagtgaccgagatgctgccacaggacctgagacactgatgaatggtgctattttggactttcaacatgctccttggcgaggtagctctgatggagttatttt

SEQ ID NO: 58 - DNA, 1024 bp, Homo sapiens
tgcagactgatatggattcaccactgctaacacctcctggttggaactacaggaatagaactggaaagggaaaaaaggcagcattcaccacatcccaatcctgaatccaagagtctaagatagtcccccactcctatctcaggcttagaggattagattaatctcctggagggaagactcttccttgaaacatttttttttatctgcctgtagctattgggataattcgggaaatccacagggacagttcaagtcatctttgtcctctactttctgttgcactctcagccttgttctctttttagaaactgcatggtaactattatatagctaaagaagagcattctgacctctgccctgggacttcctggatcctcctcttcttataaatacaagggcagagctggtatcccggggagccaggaagcagtgagcccaggagtcctcggccagccctgcctgcccaccaggaggatgaaggtctccgtggctgccctctcctgcctcatgcttgttgctgtccttggatcccaggcccagttcataaatgatgcagagacagagttaatgatgtcaaagcttccactggaaaatccagtagttctgaacagctttcactttgctgctgactgctgcacctcctacatctcacaaagcatcccgtgttcactcatgaaaagttattttgaaacgagcagcgagtgctccaagccaggtgtcatattcctcaccaagaaggggcggcaagtctgtgccaaacccagtggtccgggagttcaggattgcatgaaaaagctgaagccctactcaatataataataaagagacaaaagaggccagccacccacctccaacacctcctgagcctctgaagctcccaccaggccagctctcctcccacaacagcttcccacagcatgaagatctccgtggctgccattcccttcttcctcctcatcaccatcgccctagggaccaagactgaatcctcctcacaaactggggggaaaccgaaggttgttaa

SEQ ID NO: 59 - DNA, 1024 bp, Homo sapiens
gaaagctgtatttgctgcacgtggaaatctccgttattttccagcacccaacagtagcgtaatgggagtaacggacttaacctcatttctctttcagagcatttagccttcatatgcccttccctgcatgcttcccccaggccgtcaagacttgagttctgcctcgcttcccggcgcggtcgcagccctcagcccacttaggataatggcgacagctgaggtactgaacattggtaaaaaattatatgagggtaaaacaaaagaagtctacgaattgttagacagtccaggaaaagtcctcctgcagtccaaggaccagattacagcaggaaatgcagctagaaaaaaccacctggaaggaaaagctgcaatctcaaataaaatcaccagttgtatttttcagttattacaggaagcaggtattaaaactgccttcaccagaaaatgtggggagacagctttcattgcaccgcagtgtgaaatgattccaattgaatgggtttgcagaagaatagcaactggttcttttctcaaaagaaatcctggtgtcaaggaaggatataagttttacccacctaaagtggagttgtttttcaaggatgatgccaataatgacccacagtggtctgaggaacagctgattgctgcaaaattttgctttgctggacttcttataggccagactgaagtggatatcatgagtcatgctacacaggctatatttgaaatactggagaaatcctggttgccccagaattgtacactggttgatatgaagattgaatttggtgttgatgtaaccaccaaagaaattgttcttgctgatgttattgacaatgattcctggagactctggccatcaggagatcgaagccaacagaaagacaaacagtcttatcgggacctcaaagaagtaactcctgaagggctccaaatggtaaagaaaaactttgagtgggttgcagagagagtagagttgcttttgaaatcagaaagtcagtgcagggttgtagtgttgatgggc

SEQ ID NO: 60 - DNA, 1024 bp, Homo sapiens
agaaagccgcgcacctcctcccgccaggcgctttctcggacgccttgcccagcgggccgcccgaccccctgcaccatggaccccgctcgccccctggggctgtcgattctgctgcttttcctgacggaggctgcactgggcgatgctgctcaggagccaacaggaaataacgcggagatctgtctcctgcccctagactacggaccctgccgggccctacttctccgttactactacgacaggtacacgcagagctgccgccagttcctgtacgggggctgcgagggcaacgccaacaatttctacacctgggaggcttgcgacgatgcttgctggaggatagaaaaagttcccaaagtttgccggctgcaagtgagtgtggacgaccagtgtgaggggtccacagaaaagtatttctttaatctaagttccatgacatgtgaaaaattcttttccggtgggtgtcaccggaaccggattgagaacaggtttccagatgaagctacttgtatgggcttctgcgcaccaaagaaaattccatcattttgctacagtccaaaagatgagggactgtgctctgccaatgtgactcgctattattttaatccaagatacagaacctgtgatgctttcacctatactggctgtggagggaatgacaataactttgttagcagggaggattgcaaacgtgcatgtgcaaaagctttgaaaaagaaaaagaagatgccaaagcttcgctttgccagtagaatccggaaaattcggaagaagcaattttaaacattcttaatatgtcatcttgtttgtctttatggcttatttgcctttatggttgtatctgaagaataatatgacagcatgaggaaacaaatcattggtgatttattcaccagtttttattaatacaagtcactttttcaaaaatttggatttttttatatataactagctgctattcaaatgtgagtctaccatttttaatttatggttcaactgtttgtgagactgaattcttgcaatg

SEQ ID NO: 61 - DNA, 1024 bp, Homo sapiens
gccaccacgtgtgtccctgcgcccggtggccaccgactcagtccctcgccgaccagtctgggcagcggaggagggtggttggcagtggctggaagcttcgctatgggaagttgttcctttgctctctcgcgcccagtcctcctccctggttctcctcagccgctgtcggaggagagcacccggagacgcgggctgcagtcgcggcggcttctccccgcctgggcggccgcgccgctgggcaggtgctgagcgcccctagagcctcccttgccgcctccctcctctgcccggccgcagcagtgcacatggggtgttggaggtagatgggctcccggcccgggaggcggcggtggatgcggcgctgggcagaagcagccgccgattccagctgccccgcgcgccccgggcgcccctgcgagtccccggttcagccatggggacctctccgagcagcagcaccgccctcgcctcctgcagccgcatcgcccgccgagccacagccacgatgatcgcgggctcccttctcctgcttggattccttagcaccaccacagctcagccagaacagaaggcctcgaatctcattggcacataccgccatgttgaccgtgccaccggccaggtgctaacctgtgacaagtgtccagcaggaacctatgtctctgagcattgtaccaacacaagcctgcgcgtctgcagcagttgccctgtggggacctttaccaggcatgagaatggcatagagaaatgccatgactgtagtcagccatgcccatggccaatgattgagaaattaccttgtgctgccttgactgaccgagaatgcacttgcccacctggcatgttccagtctaacgctacctgtgccccccatacggtgtgtcctgtgggttggggtgtgcggaagaaagggacagagactgaggatgtgcggtgtaagcagtgtgctcggggtaccttctcagatgtgccttctagtgtgatgaaatgcaaagcatacacagactgtctgagtcagaacctg

SEQ ID NO: 62 - DNA, 907 bp, Homo sapiens
agatctgaaatctcattttctgtgtggctgtgtgtttgggacaggggtaaccaattcctgactactctatatgctgcatagaacctggagaggatttttcaaagtaaatgaatctcgaaagctggattgcagagcaaacgagtgcagtcaattcagccaggggcttgcaagaggagaaagagaaaaagactgtggaatggaaagtttcccaacccaagcctttcccaaggggtagccattctctgttctacagtttagggcttgcatgtgctttttctggagtccaaaaatacataagttataaccaatttaacagacagaaaggcgcacagaggaatttaaagtgtgggctggggggcgaggcggtgggcgggaggcgagcgggcgcaggcggaacaccgttttccaagctaagccgccgcaaataaaaaggcgtaaagggagagaagttggtgctcaacgtgagccaggagcagcgtcccggctcctcccctgctcattttaaaagcacttcttcttgtattgtttttaaggtgagaaataggaaagaaaacgccggcttgtgcgctcgctgcctgcctctctggctgtctgcttttgcagggctgctgggagtttttaagctctgtgagaatcctgggagttggtgatgtcagactagttgggtcatttgaaggttagcagcccgggtagggttcaccgaaagttcactcgcatatattaggcaattcaatctttcattctgtgtgacagaagtagtaggaagtgagctgttcagaggcaggagggtctattctttgccaaaggggggaccagaattcccccatgcgagctgtttgaggactgggatgccgagaacgcgagcgatccgagcagggtttgtctgggcaccgtcggggtaggatcc

SEQ ID NO: 63 - DNA, 1024 bp, Homo sapiens
atcagaaccaaattgctgagccagtcacctgtgttccaggagccgaatcagaaatgtcatcctcaggcacgccagacttacctgtcctactcaccgatttgaagattcaatatactaagatcttcataaacaatgaatggcatgattcagtgagtggcaagaaatttcctgtctttaatcctgcaactgaggaggagctctgccaggtagaagaaggagataaggaggatgttgacaaggcagtgaaggccgcaagacaggcttttcagattggatccccgtggcgtactatggatgcttccgagagggggcgactattatacaagttggctgatttaatcgaaagagatcgtctgctgctggcgacaatggagtcaatgaatggtggaaaactctattccaatgcatatctgaatgatttagcaggctgcatcaaaacattgcgctactgtgcaggttgggctgacaagatccagggccgtacaataccaattgatggaaatttttttacatatacaagacatgaacctattggtgtatgtggccaaatcattccttggaatttcccgttggttatgctcatttggaagatagggcctgcactgagctgtggaaacacagtggttgtcaaaccagcagagcaaactcctctcactgctctccacgtggcatctttaataaaagaggcagggtttcctcctggagtagtgaatattgttcctggttatgggcctacagcaggggcagccatttcttctcacatggatatagacaaagtagccttcacaggatcaacagaggttggcaagttgatcaaagaagctgccgggaaaagcaatctgaagagggtgaccctggagcttggaggaaagagcccttgcattgtgttagctgatgccgacttggacaatgctgttgaatttgcacaccatggggtattctaccaccagggccagtgttgtatagccgcatccaggatttttgtggaagaatcaatttatgatgagtttgttcgaaggagtgt

SEQ ID NO: 64 - DNA, 1024 bp, Homo sapiens
agcggggcggggcgccagcgctgccttttctcctgccgggtagtttcgctttcctgcgcagagtctgcggaggggctcggctgcaccggggggatcgcgcctggcagaccccagaccgagcagaggcgacccagcgcgctcgggagaggctgcaccgccgcgcccccgcctagcccttccggatcctgcgcgcagaaaagtttcatttgctgtatgccatcctcgagagctgtctaggttaacgttcgcactctgtgtatataacctcgacagtcttggcacctaacgtgctgtgcgtagctgctcctttggttgaatccccaggcccttgttggggcacaaggtggcaggatgtctcagtggtacgaacttcagcagcttgactcaaaattcctggagcaggttcaccagctttatgatgacagttttcccatggaaatcagacagtacctggcacagtggttagaaaagcaagactgggagcacgctgccaatgatgtttcatttgccaccatccgttttcatgacctcctgtcacagctggatgatcaatatagtcgcttttctttggagaataacttcttgctacagcataacataaggaaaagcaagcgtaatcttcaggataattttcaggaagacccaatccagatgtctatgatcatttacagctgtctgaaggaagaaaggaaaattctggaaaacgcccagagatttaatcaggctcagtcggggaatattcagagcacagtgatgttagacaaacagaaagagcttgacagtaaagtcagaaatgtgaaggacaaggttatgtgtatagagcatgaaatcaagagcctggaagatttacaagatgaatatgacttcaaatgcaaaaccttgcagaacagagaacacgagaccaatggtgtggcaaagagtgatcagaaacaagaacagctgttactcaagaagatgtatttaatgcttgacaataagagaaaggaagtagttcacaaaataatagagttgctgaatgtcactg

SEQ ID NO: 65 - DNA, 1024 bp, Homo sapiens
cgacggcggcggccgtcctcatcccggcgcttgagaggacgcggggctgcgcaaatggcttgtcccgctctcggtctggaagctcttcagcccctgcagcccgagccgccccccgagcccgccttctccgaggcgcagaagtggattgagcaagtaactggcagaagttttggtgataaagattttcggacaggtttagaaaatggaatcctcctctgcgagttgctgaatgctataaagccaggacttgttaaaaagatcaatagattgcctacccccattgcaggactggacaatattatcttattcttgagaggttgtaaagagctcggccttaaagaatctcaactttttgacccgagtgacctccaggatacatccaacagagtaacagtcaagagccttgattatagtaggaagctgaaaaatgtattagttaccatttactggctgggaaaagcagcaaacagctgcacatcctacagcggaacgacactaaacctgaaggagtttgaaggattgttggctcagatgcgaaaggacactgatgacattgaaagtcctaaacgcagtatccgagacagtggctacatcgactgctgggattccgagcgcagcgactccctctctcctcctcgccacggcagagatgattccttcgacagcctggattcctttggctctcgctctcggcagacgccttcaccagatgtagtcctcaggggaagcagcgatgggagaggaagcgactctgaatccgacttgcctcatcggaagctgccagatgtgaagaaggatgacatgtctgcacggcggacttcccatggtgagccgaaatcagcagtgccttttaaccagtacctcccgaacaaaagcaatcagacggcctacgtccccgcgcctctgagaaagaagaaagcagagagagaggaataccgcaagagctggagtaccgccacctccccgctgggtggggagaggcccttcagatacggtccgagaactcctgtgtctgatgacgcag

SEQ ID NO: 66 - DNA, 1024 bp, Homo sapiens
cgcgggccgactggtgtttatccgtcactcgccgaggttccttgggtcatggtgccagcctgactgagaagaggacgctcccgggagacgaatgaggaaccacctcctcctactgttcaagtacaggggcctggtccgcaaagggaagaaaagcaaaagacgaaaatggctaaattcgtgatccgcccagccactgccgccgactgcagtgacatactgcggctgatcaaggagctggctaaatatgaatacatggaagaacaagtaatcttaactgaaaaagatctgctagaagatggttttggagagcaccccttttaccactgcctggttgcagaagtgccgaaagagcactggactccggaaggacacagcattgttggttttgccatgtactattttacctatgacccgtggattggcaagttattgtatcttgaggacttcttcgtgatgagtgattatagaggctttggcataggatcagaaattctgaagaatctaagccaggttgcaatgaggtgtcgctgcagcagcatgcacttcttggtagcagaatggaatgaaccatccatcaacttctataaaagaagaggtgcttctgatctgtccagtgaagagggttggagactgttcaagatcgacaaggagtacttgctaaaaatggcaacagaggagtgaggagtgctgctgtagatgacaacctccattctattttagaataaattcccaacttctcttgctttctatgctgtttgtagtgaaataatagaatgagcacccattccaaagctttattaccagtggcgttgttgcatgtttgaaatgaggtctgtttaaagtggcaatctcagatgcagtttggagagtcagatctttctccttgaatatctttcgataaacaacaaggtggtgtgatcttaatatatttgaaaaaaacttcattctcgtgagtcatttaaatgtgtacaatgtacacactggtacttagagtttctgtttgattcttttttaataaactact

SEQ ID NO: 67 - DNA, 1024 bp, Homo sapiens
ctttttttctttttctttttctttttctgtttctctcccttactccttcgtctgcatcttcttctcagctgcctttccgccgttcgattcccacttccttcagaagggcgcactcttccctctgaacccctcgccgggcgtaagtgtcaggaggcggcggacgcggagattgcctcgggagcaggcgatgcgcgccgctgctccgcgcgctgcccgggggaggctggcgctagcgggcgagccggccggggctgaatggagggaaggggcgcctggattccatggggtctgcttctgtcttgaaaggaggttgggccggcgaagtggcctccctagtccccgcacacaaagctaaatggatttacctagtggattcgcggtggatgtctgtcatgtagaagtgaggaccttccaggaggagctttaaacaatttctcggccccccacccgccggcacgcactctcgcccgaaactctttgggagtatctctcgagaacctggagccctggaaatctgggagcagccacccgctccgcgcttgctgttcccggggctgcctttccggacaagcccctctccagctgcattcgcctcggctatgtttcggattgtttggggtgctaggcacggcggactccaggaccccgagaagactgtccgaaggagggagaggatgggtgccctggcgcggtgaggcggccggcccctcagcccactccgcgccgcccgctcccctgcgccctctcctctccccgcgccccaaactttgcctcccgcggcggctgcccctcggcgggcgccccgccatgtaccagaggatgctccggtgcggcgccgagctgggctcgcccgggggcggcggcggcggcggcggcggcggcggcgcaggggggcgcctggccctgctttggatagtcccgctcaccctcagcggcctcctaggagtggcgtggggggcatccagtttgggagcgcaccacatccaccatttccatggcagcagcaagcatcattcagtgccta

SEQ ID NO: 68 - DNA, 1024 bp, Homo sapiens
ctccttaggtggaaaccctgggagtagagtactgacagcaaagaccgggaaagaccatacgtccccgggcaggggtgacaacaggtgtcatctttttgatctcgtgtgtggctgccttcctatttcaaggaaagacgccaaggtaattttgacccagaggagcaatgatgtagccacctcctaaccttcccttcttgaacccccagttatgccaggatttactagagagtgtcaactcaaccagcaagcggctccttcggcttaacttgtggttggaggagagaacctttgtggggctgcgttctcttagcagtgctcagaagtgacttgcctgagggtggaccagaagaaaggaaaggtcccctcttgctgttggctgcacatcaggaaggctgtgatgggaatgaaggtgaaaacttggagatttcacttcagtcattgcttctgcctgcaagatcatcctttaaaagtagagaagctgctctgtgtggtggttaactccaagaggcagaactcgttctagaaggaaatggatgcaagcagctccgggggccccaaacgcatgcttcctgtggtctagcccagggaagcccttccgtgggggccccggctttgagggatgccaccggttctggacgcatggctgattcctgaatgatgatggttcgccgggggctgcttgcgtggatttcccgggtggtggttttgctggtgctcctctgctgtgctatctctgtcctgtacatgttggcctgcaccccaaaaggtgacgaggagcagctggcactgcccagggccaacagccccacggggaaggaggggtaccaggccgtccttcaggagtgggaggagcagcaccgcaactacgtgagcagcctgaagcggcagatcgcacagctcaaggaggagctgcaggagaggagtgagcagctcaggaatgggcagtaccaagccagcgatgctgctggcctgggtctggacaggagccccccagagaaaacccaggccgacctcctggccttcc

SEQ ID NO: 69 - DNA, 1024 bp, Homo sapiens
gtggctagtgaggcccagcctgtatatttctaacagggcgccccatgccccagcaccctccccactgggaccactcctcatagaatagccccactccaaagcatttatgacttgaaccttctgaccaaagctctctgaatcttgggggaacctagaagaggaaaaggaatgtcaggctctgtcagagtcagccaagcgagatcagccaggggtctgctccctgcagaccatacggggagccagggtttgcaatgagtctaaactggaatcttaccctgcaaaacgaatggccactattagagttttccaaaaccactctgaagcccctgactgtgcccaaagttcagatctcagaggtggatgccccgccggagggtgaccagatgccaagctccacagactccaggggcctgaagcccctgcaggaggacaccccacagctgatgcgcacacgcagtgatgttggggtgcgtcgccgtggcaatgtgaggacgcctagtgaccagcggcgaatcagacgccaccgcttctccatcaacggccatttctacaaccataagacatccgtgttcacaccagcctatggctctgtcaccaacgtccgcatcaacagcaccatgaccaccccacaggtcctgaagctgctgctcaacaaatttaagattgagaattcagcagaggagtttgccttgtacgtggtccatacgagtggtgggcccatgtgagcagatctccaaagtgttcctaatggagaaggaccaggtggaggaagtcacctacgacgtggcccagtatataaagttcgagatgccggtacttaaaagcttcattcagaagctccaggaggaagaagatcgggaagtaaagaagctgatgcgcaagtacaccgtgctccggctaatgattcgacagaggctggaggagatagccgagaccccagcaacaatctgagccatgagaacgaggggatctgggcaccccaggaaccgccattgcccataagacccccaggaagctaggcact

SEQ ID NO: 70 - DNA, 1024 bp, Homo sapiens
cggggggatcttggctgtgtgtctgcggatctgtagtggcggcggcggcggcggcggcggggaggcagcaggcgcgggagcgggcgcaggagcaggcggcggcggtggcggcggcggttagacatgaacgccgcctcggcgccggcggtgcacggagagccccttctcgcgcgcgggcggtttgtgtgattttgctaaaatgcatcaccaacagcgaatggctgccttagggacggacaaagagctgagtgatttactggatttcagtgcgatgttttcacctcctgtgagcagtgggaaaaatggaccaacttctttggcaagtggacattttactggctcaaatgtagaagacagaagtagctcagggtcctgggggaatggaggacatccaagcccgtccaggaactatggagatgggactccctatgaccacatgaccagcagggaccttgggtcacatgacaatctctctccaccttttgtcaattccagaatacaaagtaaaacagaaaggggctcatactcatcttatgggagagaatcaaacttacagggttgccaccagcagagtctccttggaggtgacatggatatgggcaacccaggaaccctttcgcccaccaaacctggttcccagtactatcagtattctagcaataatccccgaaggaggcctcttcacagtagtgccatggaggtacagacaaagaaagttcgaaaagttcctccaggtttgccatcttcagtctatgctccatcagcaagcactgccgactacaatagggactcgccaggctatccttcctccaaaccagcaaccagcactttccctagctccttcttcatgcaagatggccatcacagcagtgacccttggagctcctccagtgggatgaatcagcctggctatgcaggaatgttgggcaactcttctcatattccacagtccagcagctactgtagcctgcatccacatgaacgtttgagctatccatcacactcctcagcagacatcaattcc

SEQ ID NO: 71 - DNA, 662 bp, Homo sapiens
accgccgacgcagacccctctctgcacgccagcccgcccgcacccaccatggccacagttcagcagctggaaggaagatggcgcctggtggacagcaaaggctttgatgaatacatgaaggagctaggagtgggaatagctttgcgaaaaatgggcgcaatggccaagccagattgtatcatcacttgtgatggtaaaaacctcaccataaaaactgagagcactttgaaaacaacacagttttcttgtaccctgggagagaagtttgaagaaaccacagctgatggcagaaaaactcagactgtctgcaactttacagatggtgcattggttcagcatcaggagtgggatgggaaggaaagcacaataacaagaaaattgaaagatgggaaattagtggtggagtgtgtcatgaacaatgtcacctgtactcggatctatgaaaaagtagaataaaaattccatcatcactttggacaggagttaattaagagaatgaccaagctcagttcaatgagcaaatctccatactgtttctttcttttttttttcattactgtgttcaattatctttatcataaacattttacatgcagctatttcaaagtgtgttggattaattaggatcatccctttggttaataaataaatgtgtttgtgct

SEQ ID NO: 72 - DNA, 998 bp, Homo sapiens
cgcgaggagcaaatgtgcgcccgcacggcgagggcagcccgggcttcgctggttctcgccgaggtctttcctcgtagcgcccacactacggggaggaaaaggaatggagtcaacttcgttcgaatccaacgccagcaagtcatctttgcactcataaccacatcttttatggaacatcaagcctactctgaggtacaggctttcagaagcctgaatttggaggaccatgcctgaaccaaatggagtctccagaatcaaaactgctcaaaataatcattcatggttctcagaccaaataagatctacttagaaacatttctcgacagcacgctgccatgccgacgcagaccccactctgcacgccagcccccctgcacccaccatggccacagttcagcagctagaaggaagatggcgcctggtggacaacaaaggctttgatgaatacatgaaggagctaggagtgggaatagctttgtgaaaaatgggcgcaatggccaagccagattgtatcatcacttgtgatagcaaaaacctcaccataaaaactgagagcactttgaaaacaacacagttttctggtaccctgggagagaagtttgaagaaaacacagctgatggcagaagaactcagactgtttgcaactttacagatggtgcattggttcagcatcaggagtgggatgggaaggaaagcacaataacaagaaaactgaaagatgggaaattagtggtggagcgtgtcatgaaccatgtcgcctgtactcggatctatgaaaaagcacaataaagattccatcatcactttggacaggagttaaccaagagaatgaccaagctcagttcaatgaacaaatctccatactgtttctttcttttttttttcattactgtgttcaattatctttatcacaaacattttacatgcagctatttcaaagtgtgttggattcattaggatcatccctttggttaataaataaatgagtttctgctaa

SEQ ID NO: 73 - DNA, 1024 bp, Homo sapiens
catccttcctgtcttcgcagaggagtcctcgcgtgaaataagcgggttttgaaaacaaaaaaaagaaggagtggaagagggggccaggatccaggcctccatccccacagaagtgaagctacagctgggaggtctcctcccaccccaaccgtcaccctgggtcccgactgcccacctcctcctcctccccctccccccaacaacaacaacaacaacaactccaagcacaccggccataagagtgcgtgtgtccccaacatgaccgaacgaagaagggacgagctctctgaagagatcaacaacttaagagagaaggtcatgaagcagtcggaggagaacaacaacctgcagagccaggtgcagaagctcacagaggagaacaccacccttcgagagcaagtggaacccacccctgaggatgaggatgatgacatcgagctccgcggtgctgcagcagctgctgccccaccccctccaatagaggaagagtgcccagaagacctcccagagaagttcgatggcaacccagacatgctggctcctttcatggcccagtgccagatcttcatggaaaagagcaccagggatttctcagttgatcgtgtccgtgtctgcttcgtgacaagcatgatgaccggccgtgctgcccgttgggcctcagcaaagctggagcgctcccactacctgatgcacaactacccagctttcatgatggaaatgaagcatgtctttgaagaccctcagaggcgagaggttgccaaacgcaagatcagacgcctgcgccaaggcatggggtctgtcatcgactactccaatgctttccagatgattgcccaggacctggattggaacgagcctgcgctgattgaccagtaccacgagggcctcagcgaccacattcaggaggagctctcccacctcgaggtcgccaagtcgctgtctgctctgattgggcagtgcattcacattgagagaaggctggccagggctgctgcagctcgcaagccacgctcgccaccccggg

SEQ ID NO: 74 - DNA, 1024 bp, Homo sapiens
tataacgtgagggctgaatgcagcccattctctggagaacttcctcacacaccgcagcaaagagaagactgaaagacaaacctgggtgcagccagagaggtccagatagatgagcttgtggcatccattccccaagttcagcctagggactccacgtaccccagctgggtctcattgttccagaactgcattagttaagattacccagacttggatttcaaaggaatactttcattgttccgtctgtaacacgaagtaattggggccagctggatgtcaggatgcgtgtggttaccattgtaatcttgctctgcttttgcaaagcggctgagctgcgcaaagcaagcccaggcagtgtgagaagccgagtgaatcatggccgggcgggtggaggccggagaggctccaacccggtcaaacgctacgcaccaggcctcccgtgtgacgtgtacacatatctccatgagaaatacttagattgtcaagaaagaaaattagtttatgtgctgcctggttggcctcaggatttgctgcacatgctgctagcaagaaacaagatccgcacattgaagaacaacatgttttccaagtttaaaaagctgaaaagcctggatctgcagcagaatgagatctctaaaattgagagtgaggcgttctttggtttaaacaaactcaccaccctcttactgcagcacaaccagatcaaagtcttgacggaggaagtgttcatttacacacctctcttgagctacctgcgtctttatgacaacccctggcactgtacttgtgagatagaaacgcttatttcaatgttgcagattcccaggaaccggaatttggcgaactacgccaagtgtgaaagtccacaagaacaaaaaaataaaaaactgcggcagataaaatctgaacagttgtgtaatgaagaagaaaaggaacaattggacccgaaaccccaagtgtcagggagacccccagtcatcaagcctgaggtggactcaactttttgccacaattatgtgtttcc

SEQ ID NO: 75 - DNA, 1024 bp, Homo sapiens
atgctcctcaagaccattggcaggggcgtgttcgtcaaggtgaagctaacagggcacatactgactgggacacaggtggttacaaaaatcatctataaaatgagtggcttccctagcctctctctacagaaagaggtagaaatcatgaaggtcccgaatcacctgaacatcattaaactcaaccaggtgattggcaggtggacaccctatttagtgatggaatatgccctcgaaggagtgcttttccaccaaatacaccatcacagccacatcaaagatgacaagaaggcccgggccatgtttaagcagacaccgtccaccctccagtacagctacagaaagaaaatcgtgaactgggacctgaagccgcagaacatcctactgcatgaagaacataacttaaatatagtcaactttggtttcagcaccacatttgcagaaggagagatgctgggagccttttatgggacttgctcttatgttgccccagaactcttcctgggccatggttaccaagtccctaacaccttatttctctctctaaggcaggaatctcataagccaagatttaaatgtcatgggaaagtggaagtaatacactattctttaagaattacattctttaccatggaggggaagaaagatacttggatcctgagtcaggatttgtccccctccaaacaaccaatgtcagttggagagctgaaggctgtgacagcacgctgccacgccgacgcagaccactctctgcatgccagcccgcccgcacccaccatggccacagttcagcagctggaaggaagatggcgcctggtggacagcaaaggctttgatgaatacatgaaggagctaggagtgggaatagctttgcgaaaaatgggcgcaatggccaagccagattgtattgtcacttgtgatggcaaaaacctcaccataaaaactgagagcactttgaaaacaacacagttttcttgtcccctgggagagaagtttgaagaaaccacagctgatggcagaaaaactc

SEQ ID NO: 76 - DNA, 1024 bp, Homo sapiens
ggaaaagggaaggaggagactgagtgattaagtcacccactgtgagagctggtcttctatttaatgggggctctctctgcccaggagtcagaggtgcctccaggagcagcaggagcatggccgaggatctgggactgagctttggggaaacagccagtgtggaaatgctgccagagcacggcagctgcaggcccaaggccaggagcagcagcgcacgctgggctctcacctgctgcctggtgttgctccccttccttgcaggactcaccacatacctgcttgtcagccagctccgggcccagggagaggcctgtgtgcagttccaggctctaaaaggacaggagtttgcaccttcacatcagcaagtttatgcacctcttagagcagacggagataagccaagggcacacctgacagttgtgagacaaactcccacacagcactttaaaaatcagttcccagctctgcactgggaacatgaactaggcctggccttcaccaagaaccgaatgaactataccaacaaattcctgctgatcccagagtcgggagactacttcatttactcccaggtcacattccgtgggatgacctctgagtgcagtgaaatcagacaagcaggccgaccaaacaagccagactccatcactgtggtcatcaccaaggtaacagacagctaccctgagccaacccagctcctcatggggaccaagtctgtatgcgaagtaggtagcaactggttccagcccatctacctcggagccatgttctccttgcaagaaggggacaagctaatggtgaacgtcagtgacatctctttggtggattacacaaaagaagataaaaccttctttggagccttcttactataggaggagagcaaatatcattatatgaaagtcctctgccaccgagttcctaattttctttgttcaaatgtaattataaccaggggttttcttggggccgggagtagggggcattccacagggacaacggtttagctatgaaatttggggcccaa

SEQ ID NO: 77 - DNA, 1024 bp, Homo sapiens
agtgttgtgaagggagcagaagaggagaaacctaatctccgaacccaacaacacctagcaacagtagaaaaaacttttaaacaaaagtctgggtgggagatatacaacaacttccaatttctgctgatcgggatttcagggagaagaagctgctttccctcctctctactctgtttcctgcatgacttcagctattggctggtcctgggaaagaaagtgggaaaagctttttcctctgtattcttttgggctggatagtttttgagaactcgaatctgtcttgggctgtgagacaagccactgggttcttcaaaggataaactacctacatagaggacatacctttggttaaaggagctgccgtcaggtgggaatgagatctcgaaaccaaggtggtgaaagtgcatctgatgggcatatctcctgtcccaagccctccatcatcggcaatgctggtgaaaaaagtctctcagaagatgcaaaaaagaagaagaaatcaaataggaaggaggatgatgtcatggcctcaggaactgtcaaacgacacctaaaaacatctggagaatgtgaacgaaaaactaagaaatccctggagttatccaaagaagacctcatccaactactcagtataatggaaggggagttgcaggccagagaagatgtgatccacatgctgaagacagagaaaaccaagcctgaggttctggaggctcattacgggtctgcggagccagagaaagtgctgcgggtcctgcaccgagatgccattcttgcccaggagaaatccataggagaagatgtctatgagaaaccgatttcagagctggacagacttgaggaaaaacagaaagaaacctaccggcgcatgctagagcagctgttgctggccgagaagtgtcataggcgcaccgtatacgagttagagaacgagaagcataaacacactgactacatgaacaagagcgacgacttcaccaacctgctggagcaggagcgggagaggttaaaaaagctccttgaacaa

SEQ ID NO: 78 - DNA, 619 bp, Homo sapiens
tgcagcttccttctcaccttgaagaataatcctagaaaactcacaaaatgtgtgatgcttttgtaggtacctggaaacttgtctccagtgaaaactttgatgattatatgaaagaagtaggagtgggctttgccaccaggaaagtggctggcatggccaaacctaacatgatcatcagtgtgaatggggatgtgatcaccattaaatctgaaagtacctttaaaaatactgagatttccttcatactgggccaggaatttgacgaagtcactgcagatgacaggaaagtcaagagcaccataaccttagatgggggtgtcctggtacatgtgcagaaatgggatggaaaatcaaccaccataaagagaaaacgagaggatgataaactggtggtggaatgcgtcatgaaaggcgtcacttccacgagagtttatgagagagcataagccaagggacgttgacctggactgaagttcgcattgaactctacaacattctgtgggatatattgttcaaaaagatattgttgttttccctgatttagcaagcaagtaattttctcccaagctgattttattcaatatggttacgttggttaaataactttttttagatttag

SEQ ID NO: 79 - DNA, 1024 bp, Homo sapiens
tgggttggtgtttatctcctcccagccttgagggagggaacaacactgtaggatctggggagagaggaacaaaggaccgtgaaagctgctctgtaaaagctgacacagccctcccaagtgagcaggactgttcttcccactgcaatctgacagtttactgcatgcctggagagaacacagcagtaaaaaccaggtttgctactggaaaaagaggaaagagaagactttcattgacggacccagccatggcagcgtagcagccctgcgtttcagacggcagcagctcgggactctggacgtgtgtttgccctcaagtttgctaagctgctggtttattactgaagaaagaatgtggcagattgttttctttactctgagctgtgatcttgtcttggccgcagcctataacaactttcggaagagcatggacagcataggaaagaagcaatatcaggtccagcatgggtcctgcagctacactttcctcctgccagagatggacaactgccgctcttcctccagcccctacgtgtccaatgctgtgcagagggacgcgccgctcgaatacgatgactcggtgcagaggctgcaagtgctggagaacatcatggaaaacaacactcagtggctaatgaagcttgagaattatatccaggacaacatgaagaaagaaatggtagagatacagcagaatgcagtacagaaccagacggctgtgatgatagaaatagggacaaacctgttgaaccaaacagctgagcaaacgcggaagttaactgatgtggaagcccaagtattaaatcagaccacgagacttgaacttcagctcttggaacactccctctcgacaaacaaattggaaaaacagattttggaccagaccagtgaaataaacaaattgcaagataagaacagtttcctagaaaagaaggtgctagctatggaagacaagcacatcatccaactacagtcaataaaagaagagaaagatcagctacaggtgttagtatccaagcaaaattcc

SEQ ID NO: 80 - DNA, 1024 bp, Homo sapiens
gcaactgggggcgccccggacgaccatgagagataaggactgagggccaggaaggggaagcgagcccgccgagaggtggcggggactgctcacgccaagggccacagcggccgcgctccggcctcgctccgccgctccacgcctcgcgggatccgcgggggcagcccggccgggcggggatgccggggctggggcggagggcgcagtggctgtgctggtggtgggggctgctgtgcagctgctgcgggcccccgccgctgcggccgcccttgcccgctgccgcggccgccgccgccggggggcagctgctgggggacggcgggagccccggccgcacggagcagccgccgccgtcgccgcagtcctcctcgggcttcctgtaccggcggctcaagacgcaggagaagcgggagatgcagaaggagatcttgtcggtgctggggctcccgcaccggccccggcccctgcacggcctccaacagccgcagcccccggcgctccggcagcaggaggagcagcagcagcagcagcagctgcctcgcggagagccccctcccgggcgactgaagtccgcgcccctcttcatgctggatctgtacaacgccctgtccgccgacaacgacgaggacggggcgtcggagggggagaggcagcagtcctggccccacgaagcagccagctcgtcccagcgtcggcagccgcccccgggcgccgcgcacccgctcaaccgcaagagccttctggcccccggatctggcagcggcggcgcgtccccactgaccagcgcgcaggacagcgccttcctcaacgacgcggacatggtcatgagctttgtgaacctggtggagtacgacaaggagttctcccctcgtcagcgacaccacaaagagttcaagttcaacttatcccagattcctgagggtgaggtggtgacggctgcagaattccgcatctacaaggactgtgttatggggagttttaaaaaccaaacttttcttatcagcatttatcaagtcttacagga

SEQ ID NO: 81 - DNA, 1024 bp, Homo sapiens
cgccgcgtgcgcctgcagacgctccgctcgctgccttctctcctggcaggcgctgccttttctccccgttaaagggcacttgggctgaaggatcgctttgagatctgaggaacccgcagcgctttgagggacctgaagctgtttttcttcgttttcctttgggttcagtttgaacgggaggtttttgatccctttttttcagaatggattatttgctcatgattttctctctgctgtttgtggcttgccaaggagctccagaaacagcagtcttaggcgctgagctcagcgcggtgggtgagaacggcggggagaaacccactcccagtccaccctggcggctccgccggtccaagcgctgctcctgctcgtccctgatggataaagagtgtgtctacttctgccacctggacatcatttgggtcaacactcccgagcacgttgttccgtatggacttggaagccctaggtccaagagagccttggagaatttacttcccacaaaggcaacagaccgtgagaatagatgccaatgtgctagccaaaaagacaagaagtgctggaatttttgccaagcaggaaaagaactcagggctgaagacattatggagaaagactggaataatcataagaaaggaaaagactgttccaagcttgggaaaaagtgtatttatcagcagttagtgagaggaagaaaaatcagaagaagttcagaggaacacctaagacaaaccaggtcggagaccatgagaaacagcgtcaaatcatcttttcatgatcccaagctgaaaggcaatccctccagagagcgttatgtgacccacaaccgagcacattggtgacagaccttcggggcctgtctgaagccatagcctccacggagagccctgtggccgactctgcactctccaccctggctgggatcagagcaggagcatcctctgctggttcctgactggcaaaggaccagcgtcctcgttcaaaacattccaagaaaggttaaggagttcccccaaccatcttc

SEQ ID NO: 82 - DNA, 1024 bp, Homo sapiens
tgaaacctaacccgccctggggaggcgcgcagcagaggctccgattcggggcaggtgagaggctgactttctctcggtgcgtccagtggagctctgagtttcgaatcggcggcggcggattccccgcgcgcccggcgtcggggcttccaggaggatgcggagccccagcgcggcgtggctgctgggggccgccatcctgctagcagcctctctctcctgcagtggcaccatccaaggaaccaatagatcctctaaaggaagaagccttattggtaaggttgatggcacatcccacgtcactggaaaaggagttacagttgaaacagtcttttctgtggatgagttttctgcatctgtcctcactggaaaactgaccactgtcttccttccaattgtctacacaattgtgtttgtggtgggtttgccaagtaacggcatggccctgtgggtctttcttttccgaactaagaagaagcaccctgctgtgatttacatggccaatctggccttggctgacctcctctctgtcatctggttccccttgaagattgcctatcacatacatggcaacaactggatttatggggaagctctttgtaatgtgcttattggctttttctatggcaacatgtactgttccattctcttcatgacctgcctcagtgtgcagaggtattgggtcatcgtgaaccccatggggcactccaggaagaaggcaaacattgccattggcatctccctggcaatatggctgctgattctgctggtcaccatccctttgtatgtcgtgaagcagaccatcttcattcctgccctgaacatcacgacctgtcatgatgttttgcctgagcagctcttggtgggagacatgttcaattacttcctctctctggccattggggtctttctgttcccagccttcctcacagcctctgcctatgtgctgatgatcagaatgctgcgatcttctgccatggatgaaaactcagagaagaaaaggaagagggccatcaaactcattgtc

SEQ ID NO: 83 - DNA, 1024 bp, Homo sapiens
agcagcgagtccttgcctcccggcggctcaggacgagggcagatctcgttctggggcaagccgttgacactcgctccctgccaccgcccgggctccgtgccgccaagttttcattttccaccttctctgcctccagtcccccagcccctggccgagagaagggtcttaccggccgggattgctggaaacaccaagaggtggtttttgttttttaaaacttctgtttcttgggagggggtgtggcggggcaggatgagcaactccgttcctctgctctgtttctggagcctctgctattgctttgctgcggggagccccgtaccttttggtccagagggacggctggaagataagctccacaaacccaaagctacacagactgaggtcaaaccatctgtgaggtttaacctccgcacctccaaggacccagagcatgaaggatgctacctctccgtcggccacagccagcccttagaagactgcagtttcaacatgacagctaaaacctttttcatcattcacggatggacgatgagcggtatctttgaaaactggctgcacaaactcgtgtcagccctgcacacaagagagaaagacgccaatgtagttgtggttgactggctccccctggcccaccagctttacacggatgcggtcaataataccagggtggtgggacacagcattgccaggatgctcgactggctgcaggagaaggacgatttttctctcgggaatgtccacttgatcggctacagcctcggagcgcacgtggccgggtatgcaggcaacttcgtgaaaggaacggtgggccgaatcacaggtttggatcctgccgggcccatgtttgaaggggccgacatccacaagaggctctctccggacgatgcagattttgtggatgtcctccacacctacacgcgttccttcggcttgagcattggtattcagatgcctgtgggccacattgacatctaccccaatgggggtgacttccagccaggctgtggactcaacgatgtct

SEQ ID NO: 84 - DNA, 1024 bp, Homo sapiens
gcgcagaacttggggagccgccgccgccatccgccgccgcagccagcttccgccgccgcaggaccggcccctgccccagcctccgcagccgcggcgcgtccacgcccgcccgcgcccagggcgagtcggggtcgccgcctgcacgcttctcagtgttccccgcgccccgcatgtaacccggccaggcccccgcaactgtgtcccctgcagctccagccccgggctgcacccccccgccccgacaccagctctccagcctgctcgtccaggatggccgcggccaaggccgagatgcagctgatgtccccgctgcagatctctgacccgttcggatcctttcctcactcgcccaccatggacaactaccctaagctggaggagatgatgctgctgagcaacggggctccccagttcctcggcgccgccggggccccagagggcagcggcagcaacagcagcagcagcagcagcgggggcggtggaggcggcgggggcggcagcaacagcagcagcagcagcagcaccttcaaccctcaggcggacacgggcgagcagccctacgagcacctgaccgcagagtcttttcctgacatctctctgaacaacgagaaggtgctggtggagaccagttaccccagccaaaccactcgactgccccccatcacctatactggccgcttttccctggagcctgcacccaacagtggcaacaccttgtggcccgagcccctcttcagcttggtcagtggcctagtgagcatgaccaacccaccggcctcctcgtcctcagcaccatctccagcggcctcctccgcctccgcctcccagagcccacccctgagctgcgcagtgccatccaacgacagcagtcccatttactcagcggcacccaccttccccacgccgaacactgacattttccctgagccacaaagccaggccttcccgggctcggcagggacagcgctccagtacccgcctcctgcctaccctgccgccaagggtggcttccaggttcccatga

SEQ ID NO: 85 - DNA, 1024 bp, Homo sapiens
ttctgccctcgagcccaccgggaacgaaagagaagctctatctcgcctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaa

SEQ ID NO: 86 - DNA, 1024 bp, Homo sapiens
ttcctttcatgttcagcatttctactccttccaagaagagcagcaaagctgaagtagcagcaacagcaccagcagcaacagcaaaaaacaaacatgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttttaaaaatatcaaaacatatgaagtcctggaaaagggcatctgaaaaacctagaacaagtttaactgtgactactgaaatgacaagaattctacagtaggaaactgagacttttctatggttttgtgactttcaacttttgtacagttatgtgaaggatgaaaggtgggtgaaaggaccaaaaacagaaatacagtcttcctgaatgaatgacaatcagaattccactgcccaaaggagtccagcaattaaatggatttctaggaaaagctaccttaagaaaggctggttaccatcggagtttacaaagtgctttcacgttcttacttgttgtattatacattcatgcatttctaggctagagaaccttctagatttgatgcttacaactattctgttgtgactatgagaacatttctgtctctagaagttatctgtctgtattgatctttatgctatattactatctgtggttacagtggagacattgacattattactggagtcaagcccttataagtcaaaagcatctatgtgtcgtaaagcattcctcaaacattttttcatgcaaatacacacttctttccccaaatatcatgtagcacatcaatatgtagggaaacattcttatgcatcatttggtttgtttta

SEQ ID NO: 87 - DNA, 1024 bp, Homo sapiens
gctgcagaggattcctgcagaggatcaagacagcacgtggacctcgcacagcctctcccacaggtaccatgaaggtctccgcggcagccctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtctttgtcacccgaaagaaccgccaagtgtgtgccaacccagagaagaaatgggttcgggagtacatcaactctttggagatgagctaggatggagagtccttgaacctgaacttacacaaatttgcctgtttctgcttgctcttgtcctagcttgggaggcttcccctcactatcctaccccacccgctccttgaagggcccagattctaccacacagcagcagttacaaaaaccttccccaggctggacgtggtggctcacgcctgtaatcccagcactttgggaggccaaggtgggtggatcacttgaggtcaggagttcgagaccagcctggccaacatgatgaaaccccatctctactaaaaatacaaaaaattagccgggcgtggtagcgggcgcctgtagtcccagctactcgggaggctgaggcaggagaatggcgtgaacccgggaggcggagcttgcagtgagccgagatcgcgccactgcactccagcctgggcgacagagcgagactccgtctcaaaaaaaaaaaaaaaaaaaaaaatacaaaaattagccgggcgtggtggcccacgcctgtaatcccagctactcgggaggctaaggcaggaaaattgtttgaacccaggaggtggaggctgcagtgagctgagattgtgccacttcactccagcctgggtgacaaagtgagactccgtcacaacaacaacaacaaaaagcttccccaactaaagcctagaagagcttctgaggcgctgctttgt

SEQ ID NO: 88 - DNA, 1024 bp, Homo sapiens
cgcggtatctgcatcgggcctcactggcttcaggagctgaataccctcccaggcacacacaggtgggacacaaataagggttttggaaccactattttctcatcacgacagcaacttaaaatgcctgggaagatggtcgtgatccttggagcctcaaatatactttggataatgtttgcagcttctcaagcttttaaaatcgagaccaccccagaatctagatatcttgctcagattggtgactccgtctcattgacttgcagcaccacaggctgtgagtccccatttttctcttggagaacccagatagatagtccactgaatgggaaggtgacgaatgaggggaccacatctacgctgacaatgaatcctgttagttttgggaacgaacactcttacctgtgcacagcaacttgtgaatctaggaaattggaaaaaggaatccaggtggagatctactcttttcctaaggatccagagattcatttgagtggccctctggaggctgggaagccgatcacagtcaagtgttcagttgctgatgtatacccatttgacaggctggagatagacttactgaaaggagatcatctcatgaagagtcaggaatttctggaggatgcagacaggaagtccctggaaaccaagagtttggaagtaacctttactcctgtcattgaggatattggaaaagttcttgtttgccgagctaaattacacattgatgaaatggattctgtgcccacagtaaggcaggctgtaaaagaattgcaagtctacatatcacccaagaatacagttatttctgtgaatccatccacaaagctgcaagaaggtggctctgtgaccatgacctgttccagcgagggtctaccagctccagagattttctggagtaagaaattagataatgggaatctacagcacctttctggaaatgcaactctcaccttaattgctatgaggatggaagattctggaatttatgtgtgtgaaggagttaatttgattgggaaaaacagaa

SEQ ID NO: 89 - DNA, 1024 bp, Homo sapiens
gacagagcccgggccacggagctccttgccagctctcctcctcgcacagccgctcgaaccgcctgctgagccccatggcccgcgccacgctctccgccgcccccagcaatccccggctcctgcgggtggcgctgctgctcctgctcctggtggccgccagccggcgcgcagcaggagcgcccctggccactgaactgcgctgccagtgcttgcagaccctgcagggaattcacctcaagaacatccaaagtgtgaaggtgaagtcccccggaccccactgcgcccaaaccgaagtcatagccacactcaagaatgggcagaaagcttgtctcaaccccgcatcgcccatggttaagaaaatcatcgaaaagatgctgaaaaatggcaaatccaactgaccagaaggaaggaggaagcttattggtggctgttcctgaaggaggccctgccttacaggaacagaagaggaaagagagacacagctgcagaggccacctggcttgcgcctaatgtgtttgagcatacttaggagaagtcttctatttatttatttatttatttatttgtttgttttagaagattctatgttaatattttatgtgtaaaataaggttatgattgaatctacttgcacactctcccattatatttattgtttattttaggtcaaacccaagttagttcaatcctgattcatatttaatttgaagatagaaggtttgcagatattctctagtcatttgttaatatttcttcgtgatgacatatcacatgtcagccactgtgatagaggctgaggaatccaagaaaatggccagtaagatcaatgtgacggcagggaaatgtatgtgtgtctattttgtaactgtaaagatgaatgtcagttgttatttattgaaatgatttcacagtgtgtggtcaacatttctcatgttgaagctttaagaactaaaatgttctaaatatcccttggcattttatgtctttcttgtaagatactgccttgtttaatgttaattatgca

SEQ ID NO: 90 - DNA, 1024 bp, Homo sapiens
gtgcagaaggcacgaggaagccacagtgctccggatcctccaatcttcgctcctccaatctccgctcctccacccagttcaggaacccgcgaccgctcgcagcgctctcttgaccactatgagcctcctgtccagccgcgcggcccgtgtccccggtccttcgagctccttgtgcgcgctgttggtgctgctgctgctgctgacgcagccagggcccatcgccagcgctggtcctgccgctgctgtgttgagagagctgcgttgcgtttgtttacagaccacgcaaggagttcatcccaaaatgatcagtaatctgcaagtgttcgccataggcccacagtgctccaaggtggaagtggtagcctccctgaagaacgggaaggaaatttgtcttgatccagaagccccttttctaaagaaagtcatccagaaaattttggacggtggaaacaaggaaaactgattaagagaaatgagcacgcatggaaaagtttcccagtcttcagcagagaagttttctggaggtctctgaacccagggaagacaagaaggaaagattttgttgttgtttgtttatttgtttttccagtagttagctttcttcctggattcctcactttgaagagtgtgaggaaaacctatgtttgccgcttaagctttcagctcagctaatgaagtgtttagcatagtacctctgctatttgctgttattttatctgctatgctattgaagttttggcaattgactatagtgtgagccaggaatcactggctgttaatctttcaaagtgtcttgaattgtaggtgactattatatttccaagaaatattccttaagatattaactgagaaggctgtggatttaatgtggaaatgatgtttcataagaattctgttgatggaaatacactgttatcttcacttttataagaaataggaaatattttaatgtttcttggggaatatgttagagaatttccttactcttgattgtgggatactatttaattatttcactttagaa

SEQ ID NO: 91 - DNA, 1024 bp, Homo sapiens
aagactgcgagctccccgcaccccctcgcactccctctggccggcccagggcgccttcagcccaacctccccagccccacgggcgccacggaacccgctcgatctcgccgccaactggtagacatggagacccctgcctggccccgggtcccgcgccccgagaccgccgtcgctcggacgctcctgctcggctgggtcttcgcccaggtggccggcgcttcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaaatattctacatcattggagctgtggtatttgtggtcatcatccttgtcatcatcctggctatatctctacacaagtgtagaaaggcaggagtggggcagagctggaaggagaactccccactgaatgtttcataaaggaagcactgtt

SEQ ID NO: 92 - DNA, 623 bp, Homo sapiens
aggccaagctggactgcataaagattggtatggccttagctcttagccaaacaccttcctgacaccatgagggccagcagcttcttgatcgtggtggtgttcctcatcgctgggacgctggttctagaggcagctgtcacgggagttcctgttaaaggtcaagacactgtcaaaggccgtgttccattcaatggacaagatcccgttaaaggacaagtttcagttaaaggtcaagataaagtcaaagcgcaagagccagtcaaaggtccagtctccactaagcctggctcctgccccattatcttgatccggtgcgccatgttgaatccccctaaccgctgcttgaaagatactgactgcccaggaatcaagaagtgctgtgaaggctcttgcgggatggcctgtttcgttccccagtgagagggagccggtccttgctgcacctgtgccgtccccagagctacaggccccatctggtcctaagtccctgctgcccttccccttcccacactgtccattcttcctcccattcaggatgcccacggctggagctgcctctctcatccactttccaataaagagttccttctgctccaaaaaaaaaaaaaaaaaaaaaaaaaaaa

SEQ ID NO: 93 - DNA, 1024 bp, Homo sapiens
ggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctc

SEQ ID NO: 94 - DNA, 1024 bp, Homo sapiens
acgcgcgccctgcggagcccgcccaactccggcgagccgggcctgcgcctactcctcctcctcctctcccggcggcggctgcggcggaggcgccgactcggccttgcgcccgccctcaggcccgcgcgggcggcgcagcgaggccccgggcggcgggtggtggctgccaggcggctcggccgcgggcgctgcccggccccggcgagcggagggcggagcgcggcgccggagccgagggcgcgccgcggagggggtgctgggccgcgctgtgcccggccgggcggcggctgcaagaggaggccggaggcgagcgcggggccggcggtgggcgcgcagggcggctcgcagctcgcagccggggccgggccaggcgttcaggcaggtgatcggtgtggcggcggcggcggcggcggccccagactccctccggagttcttcttggggctgatgtccgcaaatatgcagaattaccggccgggtcgctcctgaagccagcgcggggagcgagcgcggcggcggccagcaccgggaacgcaccgaggaagaagcccagcccccgccctccgccccttccgtccccaccccctacccggcggcccaggaggctccccgcgctgcgggcgcgcactccctgtttctcctcctcctggctggcgctgcctgcctctccgcactcactgctcgcgccgggcgcgctccgccagctccgtgctccccgcgccaccctcctccgggccgcgctccctaagggatggtactgaatttcgccgccacaggagaccggctggagcgcccgccccgcggcctcgcctctcctccgagcagccagcgcctcgggacgcgatgaggaccttggcttgcctgctgctcctcggctgcggatacctcgcccatgttctggccgaggaagccgagatcccccgcgaggtgatcgagaggctggcccgcagtcagatccacagcatccgggacctccagcgactcctggagatagactccgtagggagtgaggattctttggaca

SEQ ID NO: 95 - DNA, 1024 bp, Homo sapiens
ggtggcaacttctcctcctgcggccgggagcggcctgcctgcctccctgcgcacccgcagcctcccccgctgcctccctagggctcccctccggccgccagcgcccatttttcattccctagatagagatactttgcgcgcacacacatacatacgcgcgcaaaaaggaaaaaaaaaaaaaaaagcccaccctccagcctcgctgcaaagagaaaaccggagcagccgcagctcgcagctcgcagctcgcagcccgcagcccgcagaggacgcccagagcggcgagcaggcgggcagacggaccgacggactcgcgccgcgtccacctgtcggccgggcccagccgagcgcgcagcgggcacgccgcgcgcgcggagcagccgtgcccgccgcccgggcccgccgccagggcgcacacgctcccgcccccctacccggcccgggcgggagtttgcacctctccctgcccgggtgctcgagctgccgttgcaaagccaactttggaaaaagttttttgggggagacttgggccttgaggtgcccagctccgcgctttccgattttgggggcctttccagaaaatgttgcaaaaaagctaagccggcgggcagaggaaaacgcctgtagccggcgagtgaagacgaaccatcgactgccgtgttccttttcctcttggaggttggagtcccctgggcgcccccacacggctagacgcctcggctggttcgcgacgcagccccccggccgtggatgctgcactcgggctcgggatccgcccaggtagccggcctcggacccaggtcctgcgcccaggtcctcccctgccccccagcgacggagccggggccgggggcggcggcgccgggggcatgcgggtgagccgcggctgcagaggcctgagcgcctgatcgccgcggacctgagccgagcccacccccctccccagccccccaccctggccgcgggggcggcgcgctcgatctacgcgtccggggccccgcggggccgggcccggagtcggcat

SEQ ID NO: 96 - DNA, 1024 bp, Homo sapiens
gctgctcctctgtcgagctgatcacacccacagttgagctgcgctggccagagatgcctgcccacagcctggtgatgagcagcccggccctcccggccttcctgctctgcagcacgctgctggtcatcaagatgtacgtggtggccatcatcacgggccaagtgaggctgcggaagaaggcctttgccaaccccgaggatgccctgagacacggaggcccccagtattgcaggagcgaccccgacgtggaacgctgcctcagggcccaccggaacgacatggagaccatctaccccttccttttcctgggcttcgtctactcctttctgggtcctaacccttttgtcgcctggatgcacttcctggtcttcctcgtgggccgtgtggcacacaccgtggcctacctggggaagctgcgggcacccatccgctccgtgacctacaccctggcccagctcccctgcgcctccatggctctgcagatcctctgggaagcggcccgccacctgtgaccagcagctgatgcctccttggccaccagaccatgggccaagagccgccgtggctatacctggggacttgatgttccttccagattgtggtgggccctgagtcctggtttcctggcagcctgctgcgcgtgtgggtctctgggcacagtgggcctgtgtgtgtgcccgtgtgtgtgtatgtgtgtgtgtatgtttcttagccccttggattcctgcacgaagtggctgatgggaaccatttcaagacagattgtgaagattgatagaaaatccttcagctaaagtaacagagcatcaaaaacatcactccctctccctccctaacagtgaaaagagagaagggagactctatttaagattcccaaacctaatgatcatctgaatcccgggctaagaatgcagacttttcagactgaccccagaaattctggcccagccaatctagaggcaagcctggccatctgtatttttttttttccaagacagagtcttgctctgttgcccaagctgga

SEQ ID NO: 97 - DNA, 1024 bp, Homo sapiens
actcgccgcagcctgcgcgccttctccagtccgcggtgccatggcccccgcccgtctgttcgcgctgctgctgttcttcgtaggcggagtcgccgagtcgatccgagagactgaggtcatcgacccccaggacctcctagaaggccgatacttctccggagccctaccagacgatgaggatgtagtggggcccgggcaggaatctgatgactttgagctgtctggctctggagatctggatgacttggaagactccatgatcggccctgaagttgtccatcccttggtgcctctagataaccatatccctgagagggcagggtctgggagccaagtccccaccgaacccaagaaactagaggagaatgaggttatccccaagagaatctcacccgttgaagagagtgaggatgtgtccaacaaggtgtcaatgtccagcactgtgcagggcagcaacatctttgagagaacggaggtcctggcagctctgattgtgggtggcatcgtgggcatcctctttgccgtcttcctgatcctactgctcatgtaccgtatgaagaagaaggatgaaggcagctatgacctgggcaagaaacccatctacaagaaagcccccaccaatgagttctacgcgtgaagcttgcttgtgggcactggcttggactttagcggggagggaagccaggggattttgaagggtggacattagggtagggtgaggtcaacctaatactgacttgtcagtatctccagctctgattacctttgaagtgttcagaagagacattgtcttctactgttctgccaggttcttcttgagctttgggcctcagttgccctggcagaaaaatggattcaacttggcctttctgaaggcaagactgggattggatcacttcttaaacttccagttaagaatctaggtccgccctcaagcccatactgaccatgcctcatccagagctcctctgaagccagggggctaacggatgttgtgtggagtcctggctggaggtcctccccc

SEQ ID NO: 98 - DNA, 1024 bp, Homo sapiens
ctgcttgtcaaaaggcggcagcggagccgtgtgcgccgggagcgcggaacagcttgtccacccgccggccggaccagaagcctttgggtctgaagtgtctgtgagacctcacagaagagcacccctgggctccacttacctgccccctgctccttcagggatggaggcaatggcggccagcacttccctgcctgaccctggagactttgaccggaacgtgccccggatctgtggggtgtgtggagaccgagccactggctttcacttcaatgctatgacctgtgaaggctgcaaaggcttcttcaggcgaagcatgaagcggaaggcactattcacctgccccttcaacggggactgccgcatcaccaaggacaaccgacgccactgccaggcctgccggctcaaacgctgtgtggacatcggcatgatgaaggagttcattctgacagatgaggaagtgcagaggaagcgggagatgatcctgaagcggaaggaggaggaggccttgaaggacagtctgcggcccaagctgtctgaggagcagcagcgcatcattgccatactgctggacgcccaccataagacctacgaccccacctactccgacttctgccagttccggcctccagttcgtgtgaatgatggtggagggagccatccttccaggcccaactccagacacactcccagcttctctggggactcctcctcctcctgctcagatcactgtatcacctcttcagacatgatggactcgtccagcttctccaatctggatctgagtgaagaagattcagatgacccttctgtgaccctagagctgtcccagctctccatgctgccccacctggctgacctggtcagttacagcatccaaaaggtcattggctttgctaagatgataccaggattcagagacctcacctctgaggaccagatcgtactgctgaagtcaagtgccattgaggtcatcatgttgcgctccaatgagtccttcaccatggacgacatgtcctggacctgt

SEQ ID NO: 99 - DNA, 1024 bp, Homo sapiens
gagaacggtggccggacggagagactgcgggtctgagggactggcgggcgggcgggccgagcggcgccgccgaggccgggctgggccgagcccaggagcgcccgggatgtagcgggccaccctgccgatgccacagcgcccggccgcgggcggagccggagccggagcctggggaggcggcgggggcccagagcgcagcccgcgccccccgcgcggagccaggcccgctgccgtccccgccgcccgggcccccggcatgcagccccggctgcggaggtgacactcacggaccttagccaccgccgccgccatcgccaccatggacgaacaggaggcattgaactcaatcatgaacgatctggtggccctccagatgaaccgacgtcaccggatgcctggatatgagaccatgaagaacaaagacacaggtcactcaaataggcagagtgacgtcagaatcaagttcgagcacaacggggagaggcgaattatagcgttcagccggcctgtgaaatatgaagatgtggagcacaaggtgacaacagtatttggacaacctcttgatctacattacatgaacaatgagctctccatcctgctgaaaaaccaagatgatcttgataaagcaattgacattttagatagaagctcaagcatgaaaagccttaggatattgctgttgtcccaggacagaaaccataacagttcctctccccactctggggtgtccagacaggtgcggatcaaggcttcccagtccgcaggggatataaatactatctaccagccccccgagcccagaagcaggcacctctctgtcagctcccagaaccctggccgaagctcacctccccctggctatgttcctgagcggcagcagcacattgcccggcaggggtcctacaccagcatcaacagtgagggggagttcatcccagagaccagcgagcagtgcatgctggatcccctgagcagtgcagaaaattccttgtctggaagctgccaatccttggacaggtcagca

SEQ ID NO: 100 - DNA, 1024 bp, Homo sapiens
ggagtaacagtgtcgccgccgccttcctcctcctcctctcgccgctaccgccgtcgccgccgccgcagccgccgccggtccgcgcggcctcgggtggccggagctcagcctgcgcgcgccgcgccctgtgtctccgggtggggcagaagactcgccccttgaacctcccgcggggactctccgtggtgtggcggccctggggctctttcttaatagccccggactgagtcccctccagtcgaggaccctctcctagtccactgacgagcggtggacacctgccgctgtatctcccccaaaccgagtccttgccctgctgcctcctcatacccacacggcggcagagaccttcaccatagcgttcgctcaactccagaaccttccgacctccgctagttcctgcgggcctttgcccgcttcccggtgcaccctccccgggagacacctcagacccccgacagcctgggcaggctcggtgcctgcgggtgcgttcctgatcacccctcccctcttccctccccctcatcctccattcccttgttttcaccctctgtcctctgcccgtcactccccttgtcacctcttggagccccctcctaaccagcggccagtgggtttcccataccccaggatgtgagcctctttaacctgtaatgctgtggctagcccttggcccctttcctgccatggagaaccaggtgctggtaattcgcatcaagatcccaaatagtggcgcggtggactggacagtgcactccgggccgcagttactcttcagggatgtgctggatgtgataggccaggttctgcctgaagcaacaactacagcatttgaatatgaagatgaagatggtgatcgaattacagtgagaagtgatgaggaaatgaaggcaatgctgtcatattattattccacagtaatggaacagcaagtaaatggacagttaatagagcctctgcagatatttccaagagcctgcaagcctcctggggaacggaacatacatggcctgaaggtgaatac

SEQ ID NO: 101 - DNA, 1024 bp, Homo sapiens
ggacagggcagctcaagacgctgaggtggtggctgcggcctttgaacaagtaagtgagccaccctcggagacccccgcgctggggacgggaggccggcgagcctcgggacctctgaaagccttgaggaggcgcggggacaccatggccgagcctctgaaggaggaagacggcgaggacggctctgcggagccccccgggcccgtgaaggccgaacccgcccacaccgctgcctctgtagcggccaagaacctggccctgcttaaagcccgctccttcgatgtgacctttgacgtgggcgacgagtacgagatcatcgagaccataggcaacggggcctatggagtggtgtcctccgcccgccgccgcctcaccggccagcaggtggccatcaagaagatccctaatgctttcgatgtggtgaccaatgccaagcggaccctcagggagctgaagatcctcaagcactttaaacacgacaacatcatcgccatcaaggacatcctgaggcccaccgtgccctatggcgaattcaaatctgtctacgtggtcctggacctgatggaaagcgacctgcaccagatcatccactcctcacagcccctcacactggaacacgtgcgctacttcctgtaccaactgctgcggggcctgaagtacatgcactcggctcaggtcatccaccgtgacctgaagccctccaacctattggtgaatgagaactgtgagctcaagattggtgactttggtatggctcgtggcctgtgcacctcgcccgctgaacatcagtacttcatgactgagtatgtggccacgcgctggtaccgtgcgcccgagctcatgctctctttgcatgagtatacacaggctattgacctctggtctgtgggctgcatctttggtgagatgctggcccggcgccagctcttcccaggcaaaaactatgtacaccagctacagctcatcatgatggtgctgggtaccccatcaccagccgtgattcaggctgtgggggctgagagggtg

SEQ ID NO: 102 - DNA, 1024 bp, Homo sapiens; identical to SEQ ID NO: 44

SEQ ID NO: 103 - DNA, 1024 bp, Homo sapiens
gaattcccagctctctgctcgctctgctcgcagtcacagacacttgagcacacgcgtacacccagacatcttcgggctgctattggattgactttgaaggttctgtgtgggtcgccgtggctgcatgtttgaatcaggtggagaagcacttcaacgctggacgaagtaaagattattgttgttattttttttttctctctctctctctcttaagaaaggaaaatatcccaaggactaatctgatcgggtcttccttcatcaggaacgaatgcaggaatttgggaactgagctgtgcaagtgctgaagaaggagatttgtttggaggaaacaggaaagagaaagaaaaggaaggaaaaaatacataatttcagggacgagagagagaagaaaaacggggactatggggagaaaaaagattcagattacgaggattatggatgaacgtaacagacaggtgacatttacaaagaggaaatttgggttgatgaagaaggcttatgagctgagcgtgctgtgtgactgtgagattgcgctgatcatcttcaacagcaccaacaagctgttccagtatgccagcaccgacatggacaaagtgcttctcaagtacacggagtacaacgagccgcatgagagccggacaaactcagacatcgtggagacgttgagaaagaagggccttaatggctgtgacagcccagaccccgatgcggacgattccgtaggtcacagccctgagtctgaggacaagtacaggaaaattaacgaagatattgatctaatgatcagcaggcaaagattgtgtgctgttccacctcccaacttcgagatgccagtctccatcccagtgtccagccacaacagtttggtgtacagcaaccctgtcagctcactgggaaaccccaacctattgccactggctcacccttctctgcagaggaatagtatgtctcctggtgtaacacatcgacctccaagtgcaggtaacacaggtggtctgatgggtggagacctcacgtctggtgc

SEQ ID NO: 104 - DNA, 1024 bp, Homo sapiens
gcccgcccgcgccccgaccagcccggcctcgggcagccactcaccggtgtccccgtccgcgtccttcctccccgggtcccggccatggcgctgagtgaacccatcctgccgtccttctccactttcgccagcccgtgccgcgagcgcggcctgcaggagcgctggccgcgcgccgaacccgagtccggcggcaccgacgacgacctcaacagcgtgctggacttcatcctgtccatggggctggatggcctgggcgccgaggccgccccggagccgccgccgccgcccccgccgcctgcgttctattaccccgaacccggcgcgcccccgccctacagcgcccccgcgggtggcctggtgtctgagctgctgcgacccgagctggatgcgccgctggggcccgcactgcacggccgctttctgctggcgccgcccggccgcctggtcaaggccgagccccctgaagcggacggcggcggcggctacggctgcgcccccgggctgacccgtggaccgcgcggcctcaagcgcgagggcgccccgggcccggcggcttcgtgcatgcgaggtcccgggggccgccccccgccgccgcccgacacaccgccgctcagccccgacggccccgcgcgcctgcccgcgcccggtccgcgcgcctccttcccgccgcctttcggtggccctggtttcggcgcgcccgggcccggcctgcattacgcgccgcctgcgcccccagccttcggtctcttcgacgacgcggccgccgccgcggcagccctgggcctggcgccccccgccgcccgcggtctcctcacgccgcctgcgtccccgctggagctgctggaggccaagccaaagcgcggccgccgctcttggccccgcaaacgcaccgccactcacacctgcagctacgcgggctgcggcaagacctacaccaagagttcgcatctgaaggcgcatctgcgcacgcacacaggtgagaagccctaccactgcaactgggacggctgcggctggaagtttg

SEQ ID NO: 105 - DNA, 21 bp, Homo sapiens
ccaagaguucgcatcugaatt

SEQ ID NO: 106 - DNA, 21 bp, Homo sapiens
uucagaugcgaacucuuggtg

SEQ ID NO: 107 - DNA, 21 bp, Homo sapiens
gcggcaagaccuacaccaatt

SEQ ID NO: 108 - DNA, 21 bp, Homo sapiens
uugguguaggucuugccgcag

SEQ ID NO: 109 - DNA, 21 bp, Homo sapiens
uucuccgaacgugucacgutt

SEQ ID NO: 110 - DNA, 21 bp, Homo sapiens
acgugacacguucggagaatt

(19) United States
(12) Patent Application Publication
(10) Pub. No.: US 2007/0184466 A1
(43) Pub. Date: Aug. 9, 2007

(54) Detection, generation and uses of atherosclerosis-protective endothelium

(75) Inventors: Guillermo Garcia-Cardena, Brookline, MA (US); Michael Gimbrone, Boston, MA (US); Kush Parmar, Boston, MA (US)

Correspondence Address: LAW OFFICE OF MICHAEL A. SANZO, LLC, 15400 CALHOUN DR., SUITE 125, ROCKVILLE, MD, 20855, US

(73) Assignee: The Brigham and Women's Hospital, Inc., Boston, MA (US)

(21) Appl. No.: 11/599,517

(22) Filed: Nov. 15, 2006

Related U.S. Application Data
(60) Provisional application No. 60/737,739, filed on Nov. 18, 2005.

(51) Int. Cl. C12Q 1/68; C12P 19/34; C12M 3/00

(52) U.S. Cl. 435/6; 435/91.2; 435/287.2

(57) ABSTRACT

The present invention is based upon the discovery that endothelial cells exposed to atheroprotectiveflow increase expression of the transcription factor KLF2 via a distinct flow-mediated signaling pathway and that this, in turn, modulates the activity of a series of genes that are responsible for maintaining the cells in an atherosclerosis-resistant state. By carrying out analyses to determine the extent to which endothelial cells are expressing these genes, a determination can be made concerning whether they are in a healthy state and factors can be examined for their effect on this state. In addition, the invention includes microarray plates or slides that can be used in carrying out such analyses.

### CROSS REFERENCE TO RELATED APPLICATIONS

The present application claims priority to, and the benefit of, U.S. provisional application 60/737,739, filed on Nov. 18, 2005, the contents of which is hereby incorporated by reference in its entirety.

### STATEMENT OF GOVERNMENT FUNDING

The United States Government has a paid-up license in this invention and the right in limited circumstances to require the patent owner to license others under reasonable terms as provided for by the terms of NIH Grant Nos. P50-HL56985 and RO1-HL078886.

### FIELD OF THE INVENTION

The present invention is directed to methods for identifying, generating and analyzing atherosclerosis-resistant endothelial cells. The methods are based upon the identification of a group of genes whose expression is modulated in response to increased levels of the transcription factor KLF2 resulting from the exposure of endothelial cells to atheroprotective flows. In addition, the invention is directed to microarray slides or plates that can be used in carrying out these methods.

### BACKGROUND OF THE INVENTION

Atherosclerosis is a disease characterized by the buildup of fatty deposits, plaques, within the walls of arteries. With time, the constriction caused by these plaques may lead to serious medical problems including congestive heart failure, heart attack or stroke. At present, atherosclerosis and its primary consequences (heart attacks and strokes) are the leading cause of illness and death in the United States, accounting for more deaths than all other causes combined.

Many risk factors associated with atherosclerosis have been identified and treatments are available to delay the advancement of this disease. Unfortunately, atherosclerosis is usually not diagnosed until overt clinical symptoms (e.g., angina, dizziness, etc.) are present and, at this point, the disease is often fairly far advanced. In many cases, there are no symptoms at all to warn patients of an impending stroke or heart attack.

Certain vascular regions are known to be especially prone to the development of atherosclerotic plaques and it is thought that this is the result of pathological changes in vascular endothelial cells. These cells comprise an interface between the blood and the other tissues of the body and play a fundamental role in the health of the cardiovascular system. The endothelium mediates processes as diverse as blood vessel formation, organogenesis, vascular tone, blood coagulation, and metabolism; and its dysfunction can contribute to chronic inflammation, hypertension, thrombosis, and atherosclerosis. The functional plasticity of this single-cell-thick layer relies on the ability of individual endothelial cells to integrate and transduce both humoral and biomechanical stimuli in their microenvironment (Gimbrone, Ann. NY Acad. Sci 902:230-239 (2000); Traub, et al., Arterioscler. Thromb. Vasc. Biol. 18:677-685 (1998)). For example, endothelial cells are able to react to inflammatory mediators by expressing cell adhesion molecules and chemokines in the context of a developing inflammatory reaction. In addition, endothelial cells are able to sense hemodynamic forces generated by the pulsatile flow of blood, and respond rapidly by secreting or metabolizing potent vasoactive substances, or chronically by regulating transcriptional programs that lead to the modulation of their functional phenotype in normal and pathological states (Davies, Physiol. Rev. 75:519-560 (1995)).

Several studies have established that distinct types of shear stresses associated with blood flow correlate with vascular regions protected from, or susceptible to, the development of atherosclerotic disease (Gimbrone, Ann. NY Acad. Sci 902:230-239 (2000)). Nevertheless, the mechanisms underlying the coordinated regulation of specific mechano-activated transcriptional programs leading to the atheroprotected and atherosusceptible endothelial phenotypes remain incompletely understood.

### SUMMARY OF THE INVENTION

The present invention is based upon experiments in which endothelial cells are cultured in the presence of culture medium and exposed to atheroprotective shear stress waveforms that mimic blood flow in atherosclerosis-resistant regions of human arteries. It has been found that the transcription factor KLF2 is activated in these cells by a distinct signaling pathway and, in turn, this transcription factor modulates the expression of a group of genes that maintain the cells in a healthy, atherosclerosis-resistant state. When KLF2 activity is blocked in endothelial cells exposed to atheroprotective flow, the cells assume a pathological, atherosclerosis-prone state.

Using total genomic microarrays, the inventors have identified the genes whose expression is activated or inhibited in response to increased KLF2 levels. It is believed that the way in which KLF2 is activated is an important factor in determining its subsequent effect on gene activity. Thus, the genes activated or inhibited when KLF2 levels increase in response to flow-induced stress may be different from those affected when KLF2 levels increase due to some other factor, e.g., due to the exposure of cells to statins. In this regard, it is possible that flow may activate other factors influencing KLF2 activity or may lead to subtle conformational changes in chromatin leading to the activation of specific genetic programs.

The results obtained suggest that flow stress induced the activation of four genes MEKK3, MEK5, Erk5, and MEF2 (Table 4) and these, in turn, upregulate the expression of KLF2 and exert a regulatory effect on genes shown in Table 3. The genes in Table 3 have been given sequence identification numbers (SEQ ID NO:1-SEQ ID NO:98) and the regulatory genes MEKK3, MEK5, Erk5, and MEF2A. MEF2C and KLF2 have been given the sequence identification numbers SEQ ID NO:99- SEQ ID NO104 respectively (see Table 4). Certain of the genes in Table 3, SEQ ID NO:1-SEQ ID NO:52, exhibit increased expression in response to KLF2, whereas others, SEQ ID NO:53-SEQ ID NO:83, exhibit reduced activity. The genes designated as SEQ D NO:84-98 are listed because changes in their activity are related to inflammation which would be expected to be present during atherosclerotic plaque formation.

The cellular changes observed as a result of atheroprotective flow should also be reflected in corresponding changes in the medium surrounding the cells as the result of cell lysis or the release of naturally secreted gene products. Thus, in its first aspect, the invention is directed to a method of examining a subject, either a human or animal, for the presence of endothelial cells that makes that subject prone to the development of atherosclerotic plaques. The test may be used diagnostically or as part of a study designed to assess how various factors, e.g., dietary factors or drugs, affect the state of endothelial cells.

The method involves assaying a test sample of blood, serum or plasma obtained from the subject to determine the level of at least one marker gene sequence as shown as SEQ ID NO:1-SEQ ID NO:104. For the purposes of the present invention, assays for determining the level of expression of a gene sequence may be directed either at nucleic acids (e.g., using PCR amplification of mRNA) or at gene products (e.g., using an ELISA or radioimmunoassay). The results obtained using the test sample are compared with results from one or more control samples selected using criteria well known in the art. The control samples may be, for example, samples of blood, serum or plasma derived from individuals known to be free of atherosclerotic plaques or they may be taken from the population as a whole and, optionally, matched with the test sample with respect to the age of the subject, sex, etc. By comparing the results from the controls and the test sample, a conclusion can be drawn with regard to whether the subject is at increased risk of plaque formation based upon whether the marker gene sequence in the test sample is significantly higher or lower than in the control samples. The exact nature of the comparison will depend upon the particular marker used. For example, the expression or activity of markers of SEQ ID NO:1-SEQ ID NO:52 and SEQ ID NO:99-SEQ ID NO:104 are increased in response to flow-activated KLF2. Thus, relatively low levels of these gene markers is indicative of cells with a tendency to form atherosclerotic plaques. In contrast, the genes of SEQ ID NO:53-SEQ ID NO:83 are decreased in response to flow-activated KLF2 and a higher level of these markers in test samples compared to control samples is indicative of a normalstate. The more marker genes exhibiting differences, the greater the risk of a subject developing atherosclerosis. Similar approaches can be used with genes that are expressed at the endothelial cell surface whose expression can be detected via non-invasive imaging modalities (e.g., ultrasound, MRI, bioluminescence, etc.).

In another aspect, the invention is directed to determining whether test endothelial cells are in a healthy (atherosclerosis-resistant) state or a pathological (atherosclerosis-prone) state. This method will be of value to scientists characterizing endothelial cells grown in vitro or obtained at autopsy or biopsy. The method involves assaying one or more of the marker genes shown as SEQ ID NO:1-SEQ ID NO:104, and comparing the results obtained with those from control endothelial cells. The control cells may, for example, be cells that are known to be in a healthy state or they may represent some other type of control selected by those of skill in the art. A low level of a marker gene that is normally increased in response to flow activated KLF2 (SEQ ID NO:1-SEQ ID NO:52, SEQ ID NO:99-SEQ ID NO:104) or a high level of a gene that is normally decreased in response to flow activated KLF2 (SEQ ID NO:53-SEQ ID NO:83) suggests endothelial cells that are in a state prone to atherosclerotic plaque formation. In contrast, gene markers that normally increase in response to flow-activated KLF2 which are at the same or higher level in test endothelial cells compared to control endothelial cells is an indication of cells that are in a healthy state. A similar conclusion may be drawn with respect to comparable or decreased levels of markers that are reduced in response to flow-activated KLF2. If desired, a comparison between test and control endothelial cells may also involve an examination of KLF2 levels (SEQ ID NO:104). Assays may involve the use of the polymerase chain reaction to amplify mRNA in test and/or control cells and, in cases where multiple markers are being examined, may be performed using a microarray plate.

Another aspect of the invention is directed to the use of the flow-mediated signaling pathway to achieve via this “molecular bluprint” the increase in the expression and activation of KLF2. This pathway includes the sequential activation of the kinases MEKK3, MEK5, Erk5 leading to the activation of members of the MEF family of transcription factors that bind and transactivate the KLF2 promoter.

In another aspect, the invention is directed to a method of assaying a test compound for its tendency to either induce endothelial cells to assume a state in which they are prone to atherosclerotic plaque formation or for a tendency to maintain endothelial cells in a healthy (atherosclerosis resistant) state. This is accomplished by contacting endothelial cells with the test compound and then measuring the expression of a marker gene sequence selected from SEQ ID NO:1-SEQ ID NO:104. The results obtained are then compared with results from one or more similar measurements performed in the absence of the test compound. A conclusion is then drawn as to whether the test compound induces endothelial cells to assume a state prone to atherosclerosis formation or tends to maintain the cells in a healthy state. In addition to the marker gene sequences shown in Table 3, measurements may also be performed of KLF2 levels both in the presence and absence of the test compound. Compounds that induce a change in the level of a marker gene similar to the change induced by flow-induced KLF2 (especially in cases where KLF2 itself is also increased) is an indication of a compound that is acting to maintain endothelial cells in a healthy state. These compounds would have potential value as therapeutic agents. Test compounds that cause a change in marker genes that is the opposite of that caused by flow-activated KLF2 (i.e., a decrease in marker genes of SEQ ID NO:1-SEQ ID NO:52 or SEQ ID NO:99-SEQ ID NO:104 or an increase in the marker genes of SEQ ID NO:53-SEQ ID NO:83) suggests that the test compound is having an adverse effect on endothelial cell health, i.e., making them atherosclerosis-prone. As a general rule, it would probably be advisable for individuals to avoid these compounds, especially individuals with cardiovascular disease.

The invention also includes methods of assaying test compounds for their tendency to induce endothelial cells exposed to shear stress to either assume an atherosclerosis prone state or to maintain a healthy state. The main characteristic of these assays is that they are performed on endothelial cells which are cultured under conditions in which culture medium flows over the cells with a flow pattern that is either characteristic of atherosclerosis-resistant arterial regions or, preferably, characteristic of atherosclerosis-prone arterial regions. The assays are performed essentially as described above and comparisons are made between gene marker or KLF2 levels both in the presence and absence of the test compound. Analysis may involve PCR amplifying mRNA from cells and performing a microarray analysis using a plate or slide having immobilized oligonucleotides which hybridize to marker gene sequences or the sequence for KLF2.

In another aspect, the invention is directed to a microarray plate or slide having a series of distinct, immobilized oligonucleotides recognizing the sequences of SEQ ID NO:1-98 and/or SEQ ID NO:99-104. The term “distinct” indicates that the oligonucleotides have different sequences that allow them to hybridize to different complementary sequences. Many methods are known in the art for producing plates or slides of this nature and any of these methods are compatible with the present invention. The plates or slides must include immobilized oligonucleotides that hybridize under stringent conditions to at least one gene sequence shown as SEQ ID NO:1-SEQ ID NO:104, and, preferably, slides include several distinct oligonucleotides binding to different marker gene sequences. The term “stringent conditions” indicates conditions that essentially only permit hybridization to occur with the exact complementary sequence of the immobilized oligonucleotide. In general, these hybridizations are performed in buffers of about neutral pH containing 0.1-0.5 NaCl and at a temperature of between 45 and 70° C. It is also possible to carry out incubations under conditions of low stringency and then to use high stringency wash conditions to cause the dissociation of hybridized sequences that are not exact matches. Procedures for carrying out incubations of this type in connection with microarray plates or slides are well known in the art.

Each group of immobilized oligonucleotides hybridizing to a specific gene marker will occupy a separate location on the microarray plate or slide and in total, there should be no more than 500 distinct oligonucleotides present. In preferred embodiments, there are at least 10 distinct oligonucleotides immobilized on plates that hybridize under stringent conditions to different marker genes with 30, 50 or 76 such immobilized oligonucleotides being preferred. For economic reasons, it is also preferred that the total number of immobilized sequences present be less than 200 or, more preferably, less than 100 sequences. In addition to having oligonucleotides recognizing the marker genes in Table 3, they may also, optionally include a sequence recognize KLF2, SEQ ID NO:104.

The microarray plates described above may be used in carrying out any of the methods of analyzing endothelial cells discussed herein. Typically, the microarray assays will involve lysing endothelial cells and then amplifying the mRNA released in the presence of a detectable label, e.g., a nucleotide bound to a dye or other marker and present in a PCR primer. Thus, a population of labeled cDNAs is obtained that can be used directly in hybridizations with oligonucleotides immobilized on a microarray plate or slide. It is also possible to compare two different populations of mRNAs by carrying out PCR in the presence of different dyes for each population. After hybridizations are completed, plates are analyzed using an automated reader, to determine the amount of label associated with each immobilized sequence which in turn reflects the abundance of the hybridized sequence in the original mRNA lysate. Many variations of this basic procedure have been described in the art and are compatible with the present invention.

### DETAILED DESCRIPTION OF THE INVENTION

I. Cells and Flow Systems

Endothelial cells used in the present invention may be either obtained from a biological sample, from a primary culture, e.g., human umbilical vein endothelial cells may be used, or from an established endothelial cell line. The culturing of these cells in systems reproducing flow patterns characteristic of atherosclerosis-prone and atherosclerosis-resistant arterial regions is carried out as described previously (Dai, et al., Proc. Natl. Acad. Sci. USA 101:14871-14876 (2004); Blackman, et al., J. Biomech. Eng. 124:397-407 (2002)).

II. Preparation of Samples for Use in Hybridizations

To analyze the extent to which gene markers or KLF2 have been expressed, endothelial cells are collected, lysed to obtain mRNA and amplified by PCR. Primers suitable for amplifying all marker gene sequences, as well as KLF2, are commercially available (e.g., from Applied Biosystems) and methods for performing PCR amplification using these primers are described by the manufacturer and as well as in the art (Dai, et al., Proc. Natl. Acad. Sci. USA 101:14871-14876 (2004)). Typically, PCR primers will be fluorescently labeled to produce an amplification product that can be detected and quantitated.

III. Microarray Materials and Assays

All of the gene markers identified herein as being modulated by flow-induced KLF2 were present on the Applied Biosystems plates described in the Examples section. In principle, the same plates could be used for evaluating the state of endothelial cells. However, since the plates used include immobilized oligonucleotides for over 27,000 different genes, the system is inconvenient and unnecessarily expensive. Plates better suited to endothelial cell analysis as discussed herein can be made by focusing on the 76 genes listed in Table 3. Thus, plates similar to the Applied Biosystems plates may be used, under the same assay conditions, but with only a small number of hybridization sites (e.g., 10-100). This would simplify the analysis and allow for replicates to be included to better check on the consistency of results.

Although the same procedures and hardware described by Applied Biosystems could be employed in connection with the present invention, other alternatives are also available. Many reviews have been written detailing methods for making microarrays and for carrying out assays (see, e.g., Bowtell, Nature Genetics Suppl. 21:25-32 (1999); Constantine, et al., Life Sci. News 1:11-13 (1998); Ramsay, Nature Biotechnol. 16:40-44 (1998)). In addition, patents have issued describing techniques for producing microarray plates, slides and related instruments (U.S. Pat. No. 6,902,702; U.S. Pat. No. 6,594,432; U.S. Pat. No. 5,622,826) and for carrying out assays (U.S. Pat. No. 6,902,900; U.S. Pat. No. 6,759,197). The two main techniques for making plates or slides involve either polylithographic methods (see U.S. Pat. No. 5,445,934; U.S. Pat. No. 5,744,305) or robotic spotting methods (U.S. Pat. No. 5,807,522). Other procedures may involve inkjet printing or capillary spotting (see, e.g., WO 98/29736 or WO 00/01859).

The substrate used for microarray plates or slides can be any material capable of binding to and immobilizing oligonucleotides including plastic, metals such a platinum and glass. A preferred substrate is glass coated with a material that promotes oligonucleotide binding such as polylysine (see Chena, et al., Science 270:467-470 (1995)). Many schemes for covalently attaching oligonucleotides have been described and are suitable for use in connection with the present invention (see, e.g., U.S. Pat. No. 6,594,432). The immobilized oligonucleotides should be, at a minimum, 20 bases in length and should have a sequence exactly corresponding to a segment in the gene targeted for hybridization.

IV. Comments Regarding Additional Methodology

Although the methods described above may be used to determine the levels of marker genes and KLF2 in endothelial cells, any other procedure for conducting this analysis may also be used in connection with the invention. For example, DNA blotting techniques, with or without PCR amplification, may be used to quantitate levels of KLF2 or marker genes. Western blots or immunoassays may be used to quantitate gene products and, in some cases, enzyme-based assays may be used. The levels of genes can also be assessed by immunofluorescence techniques or promoter based reporter assays. The essential element of the procedure is not how quantitation is performed, but rather the particular genes being examined and the determination of whether those genes are being expressed at a level characteristic of atherosclerosis-prone or atherosclerosis-resistant cells.

V. Comments Regarding Utility

The assays described herein are designed to assess whether endothelial cells are in a state that makes them particularly resistant to the formation of atherosclerotic plaques. This is clearly of great value to scientists that are studying cardiovascular diseases. For example, the ability to assess the state of cells in experimental animal models of atherosclerosis will help to pinpoint regions where problems exist and to assess whether a drug regimen is having a beneficial effect. The cells may also be used in the development of new diagnostic assays that can identify individuals likely to develop cardiovascular problems before clinical symptoms. As mentioned previously, the way in which KLF2 activity is induced is of great importance in determining the genes that it will subsequently modulate. Thus, assays that are performed in systems that allow for the mimicking of blood flow patterns in atherosclerosis-resistant arterial regions will be of particular value in the development and identification of new drug agents. In addition the atherosclerosis-prone phenotype can be used as a background against to which drug screening can be performed to “rescue” that phenotype. This system will also be of great help in identifying factors (dietary substances or drugs) that may be contributing to the disease process.

Finally, the microarray plates that have been discussed herein will allow for the assessment of an entire array of genes that are involved in protecting endothelial cells. While it is possible to draw conclusions based upon the levels of a single marker or KLF2, it is obviously much better to examine an entire array of factors affecting the state of cells. Although it is possible to quantitate the marker genes and KLF2 using genomic microarray plates such as those sold by Applied Biosystems, Agilent, or Affymetrix it would be simpler and more economical to use plates that are designed specifically for this purpose and which have selectively immobilized oligonucleotides hybridizing with the marker genes described herein.

### EXAMPLES

In the present example, evidence is presented identifying the transcription factor KLF2 as a key regulator of multiple endothelial functions that is both, necessary and sufficient, to evoke atheroprotective transcriptional programs and functional phenotypes in endothelial cells exposed to a hemodynamic environment characteristic of athero-resistant regions of the human carotid artery. In addition, many of the factors induced or inhibited by flow-induced KLF2 are identified.

I. Methods

Cell culture and Dynamic Flow System

Human umbilical vein endothelial cells (HUVEC) were isolated and cultured as previously described (Dai, et al., Proc. Nat'l Acad. Sci. USA 101:14871-14876 (2004)). Cells were exposed to “athero-protective” and “athero-prone” shear stress waveforms in a DFS as described (Dai, et al., Proc, Nat'l Acad. Sci. USA 101:14871-14876 (2004); Blackman, et al., J. Biomech. Eng. 124:397-407 (2002)).

RNA Processing and Transcription Profiling

RNA isolation and quantitative RT-PCR was performed as previously described (Dai, et al., Proc. Nat'l Acad. Sci. USA 101:14871-14876 (2004)). All primers used were obtained from Applied Biosystems. For microarray analysis, HUVEC were plated at 7×10⁴ cells/cm² in 100 cm² plates, cultured for 24 hours, and then infected with Ad-GFP or Ad-KLF2 for 24 hours. In some samples, cells were incubated with 10 U/mL IL-1 for 4 hours. Applied Biosystems total genome oligo-based microarrays containing ˜27,868 unique genes were used in all experiments. Array hybridization was performed according to the manufacturer's instructions using 40 μg of total RNA. Array images were processed using the Applied Biosystems 1700 Array Scanner software, to generate spot quantifications and exclude poor quality spots. Genes differentially regulated between any two conditions (X and Y) were detected based on three pairs of replicate arrays for condition X and condition Y. Spot measurements between paired arrays for each comparison were normalized between conditions using Lowess (Yang, et al., Nucl. Ac. Res. 30:e15 (2002)). For both array systems, a minimum intensity-based variance estimation algorithm was used to determine p-values for genes whose differential regulation between conditions was statistically significant (Comander, et al., BMC Genomics 5:17 (2004)). Spots with p-values less than 0.05 were considered to be significantly regulated.

GEDI Clustering

Self-organizing maps were generated from transcriptional profiling data using software described elsewhere (Eichler, et al., Bioinformatics 19:2321-2322 (2003)) and available at: http://www.chip.org/˜ge/gedihome.html. Only genes that were regulated at least twofold in at least one condition relative to control (Ad-GFP) were included in map generation. Definitions of gene expression patterns contained within the indicated cluster regions were the following:
I) |IL-1 exp/GFPexp|<2, IL-1 exp/GFPexp>2 KLF2exp/GFPexp, IL-1 exp/GFPexp>2 KLF2+IL-1exp/GFPexp;
II) KLF2exp/GFPexp<2, IL-1 exp/GFPexp>23/2, 2 KLF2+IL-1 exp/GFPexp<IL-1/GFPexp;
III) 2 KLF2exp/GFPexp<KLF2+IL-1 exp/GFPexp, 2 IL-1/GFPexp<KLF2+IL-1 exp/GFPexp, KLF2exp/GFPexp and IL-1 exp/GFPexp do not differ by more than twofold;
IV) |IL-1 exp/GFPexp|<2, KLF2exp/GFPexp>2, KLF2+IL-1 exp/GFPexp>2
   where the expression of a gene, for example, in a condition X, is denoted by Xexp. The four conditions (GFP, KLF2, IL-1, KLF2+IL-1) are as described above.

Western Blotting

Cells were washed once with ice cold PBS and lysed in boiling sample buffer containing beta-mercaptoethanol. The crude lysates were then boiled for 5 minutes, centrifuged for 10 minutes at 4° C., and supernatants were loaded for SDS-PAGE. Protein was transferred to a nitrocellulose membrane, which was blocked with 5% milk in TBS-T (125 mM NaCl, 25 mM Tris pH8.0, 0.05% Tween 20), incubated with primary antibody in 1% milk/TBST for 1 hr at RT (Tie2, GAPDH, α-tubilin) or overnight at 4 degrees (eNOS), followed by 4 15-minute washes with TBST. Secondary antibody was then added at a 1:5000 dilution for 1 hr in 1% milk/TBST, followed by an 4 washed in TBST and then addition of equal amounts of endothelial cell reagents (Pierce). Monoclonal antibody to human Tie2 was from R&D Systems (mAb clone 83711), monoclonal antibody to GAPDH was from Abcam (clone 6C5), polyclonal antibody to a-tubilin was from Santa Cruz Biotechnology. The H32 anti-eNOS monoclonal antibody is as described (Sessa, et al, J. Biol. Chem. 270:17641-17644 (1995)).

FACS Analysis

For FACS analysis, cells were plated in 6-well plates, infected with either Ad-GFP or Ad-LKLF encoding viruses for 24 hours, then incubated with media or 10 Units/mL IL-1β for 8 hours. Cells were then washed in cold PBS once, resuspended in PBS, and centrifuged for 5 min. Each sample was then resuspended in 1 mL of 5% FBS in PBS (staining buffer), after which 1 μg of anti-CD141 mAb (BD Biosciences) or 1 μg of TF antibody (American Diagnostica) per million cells was added, following a 30 min incubation on ice. Cells were then spun down and resuspended in staining buffer. APC-conjugated anti-mouse IgG1 was added at 0.5 μg/million cells for 30 min on ice. Cells were then washed twice in staining buffer, following fixation with a 1:1 mixture of staining buffer:1% PFA/PBS. Cells were processed using a FACScalibur apparatus (BD Biosciences) using CellQuest software.

aPC Chromogenic Assay

HUVEC were plated at 7×10⁴ cells/cm2 in 24-well plates for 24 hours, followed by addition of IL-1β (10 U/mL) or normal media for 8 hours before the chromogenic assay. The cells were washed once with PBS and then overlaid with 500 μl Hanks balanced salt solution (HBSS) supplemented with 1 mM magnesium and calcium in 20 mM HEPES buffer (pH 7.4 with 0.1% BSA. 10 μg/mL human protein C (Enzyme Research Laboratories, South Bend, Ind.) was added followed by 1 nM human thrombin (Sigma, St. Louis, Mo.). Cells were incubated under normal culture conditions for 2 hours, after which the reaction was terminated with 100 nM hirudin (Sigma). Plates were centrifuged and supernatant fluid was aspirated. APC was quantitated from the supernatant by addition of 500 μM S-2366 chromogenic substrate and measured at 405 nm at the indicated time points. Non-TM dependent APC signal was determined from wells without cultured HUVEC, and this level was subtracted from the experimental results. Total protein levels as determined by BSA quantitation kit were not altered in any of the conditions.

Protein Array and Multiplex ELISA

The Zyomyx Cytokine Array was used to measure analytes (IL-6) in the supernatant as per the protocol of the company. Multiplex ELISAs were performed using the Cytometric Bead Array (CBA Kit, BD Pharmingen) Chemokine Kit I and Inflammation Kit. To obtain samples, HUVEC were infected with Ad-GFP or Ad-KLF2 for 24 hours, followed by incubation in media with or without 10 U/mL IL-1β for 8 hours. Supernatants were then isolated, centrifuged for 20 min to pellet debris, aliquoted and frozen at −80° C. Supernatants were analyzed using the CBA Kits as instructed, using a FACScalibur (BD Biosciences) apparatus and data analysis with the CBA Analysis Software.

An-2, 15-d-PGJ2, and CNP ELISA

The Ang-2 ELISA Kit (R&D Systems) was used as per directions of the manufacturer. Normal media was used as a baseline control to subtract from the values of conditioned media. 15d-PGJ2 was measured in media using an ELISA Kit (R&D Systems) as instructed by the manufacturer. Prior to analysis, prostaglandin was acid extracted on a reverse phase column from 2 mL supernatant. Samples were acidified to ˜pH 3.0 by addition of 100 μL 2N HCl followed by incubation at 4 degrees for 15 minutes. Samples were cleared by centrifugation at RT for 2 minutes. Amprep C-18 500 μg minicolumns (Amersham Biosciences) were equilibrated by washing with 10 volumes (28 mL) of ethanol followed by 10 volumes of water. Samples were then applied using slight positive pressure (flow rate ˜0.5 mL/min), and the columns were washed with 28 mL of water, 28 mL 15% ethanol, and 28 mL hexane. Prostaglandins were then eluted with 20 mL ethyl acetate. The samples were then evaporated under a stream of nitrogen; 50 μl was added to the dried samples, followed by reconstitution with 200 μL of assay buffer provided in the kit. CNP was measured using an ELISA kit (Peninsula Laboratories) as instructed by the manufacturer.

siRNA Design and Preparation

The KLF2 siRNAs were designed based on the characterization of siRNA by Elbashir et al. (Embo J. 20:6877-6888 (2001)) and were synthesized by Qiagen (Valencia, Calif.). Two designs with the highest score were chosen. The sense and antisense strands of human KLF2 siRNA were: (a) 5′-CCAAGAGUUCGCATCUGAATT-3′ (sense, SEQ ID NO:105) and 5′-UUCAGAUGCGAACUCUUGGTG-3′ (antisense, SEQ ID NO:106); (b) 5′-GCGGCAAGACCUACACCAATT-3′ (sense, SEQ ID NO:107) and 5′-UUGGUGU AGGUCUUGCCGCAG-3′ (antisense, SEQ ID NO:108). Negative control siRNA were 5′-UUCUCCGAACGUGUCACGUTT-3′ (sense, SEQ ID NO:109) and 5′-ACGUGACACG UUCGGAGAATT-3′ (antisense, SEQ ID NO:110). For some experiments, the 3′ end of the sense strand was labeled with Alexa Fluor 488 (Qiagen) and used to determine the transfection efficiency. Transfection efficiency of over 95% was consistently achieved. siRNA aliquots were heated to 95 degrees for 2 minutes and then incubated at 37 degrees for 1 hour to allow annealing before transfection.

Transfection of siRNA Duplexes into HUVEC

HUVEC were plated 16 hours prior to transfection at a density of 40,000 cells/cm² in HUVEC culture medium without antibiotics. At the time of transfection with siRNA, the cells were 85-95% confluent. Individual siRNAs (KLF2 siRNA a & b, 6 nmol each, or negative control siRNA, 12 nmol) were mixed with Oligofectamine (80 μl) and Opti-MEM (Invitrogen) in a final volume of 2 ml and incubated at room temperature for 15 min for the lipid-siRNA complex to form. Cells were washed once and 8 ml Opti-MEM was added. The 2 ml Oligofectamine-siRNA complex solution was then added to the cells. The cells were then incubated for 6 hour at 37° C. Next, 5 ml HUVEC culture medium was added. Flow experiments were performed 24 hours after the siRNA transfection.

Interferon response was monitored in every sample for each siRNA experiment by RT-PCR to the dsRNA-responsive genes 2′-5′-oligoadenylate synthetase (OAS2) and interferon-induced transmembrane protein 1 (IFITM1) (Sledz, Nat. Cell Biol. 5:834-839 (2003)). 10 μg/mL Poly(I)-Poly(C) dsRNA (Sigma) was used as a positive control for induction of the interferon response. No upregulation of the two genes tested was found in any of the siRNA transfection experiments.

Leukocyte Adhesion

1 million HL-60 cells were spun down and washed with warm PBS (no calcium), pelleted, and resuspended and incubated at 37° C. with Medium 199 plus 10 μM Cell Tracker Green (Molecular Probes) for 20 min. The cells were pelleted, washed with PBS, and resuspended in warm HUVEC Media (0.2×10⁶ cells/mL). They were then allowed to recover 15 min at 37° C. HUVEC on a fragment of the plastic surface placed under shear were washed twice with PBS to remove cell debris, and the 5 mL of HL-60 suspension (1 million cells total) was added to the plastic piece of the shear plate within a 100 mm dish. The dish was place on a horizontal rotator at ˜60 rpm for 10 min at 20° C. to allow binding. Monolayers were then washed gently with PBS three times simply by exchanging the media, replaced with HUVEC media, and then visualized under a microscope under brightfield and 488 filter (to visualize labeled HL-60 cells. For quantification of bound HL-60s, all the cells were lysed with 200 μL lysis buffer (0.1% sodium hydroxide/0.01% SDS); 100 μL was loaded onto 96-well black plate for quantification of fluorescence (Excitation/Emission=492/517).

II. Results

To identify critical transcriptional regulators of atheroprotective mechano-activated programs, and thus gain mechanistic insights into the molecular basis of the atheroprotected vs. atherosusceptible endothelial phenotypes, we have recreated the shear stresses characteristic of the atheroprotected (athero-protective waveform) or atherosusceptible regions (athero-prone waveform) of the human carotid artery bifurcation, using an in vitro system recently developed in our laboratory (Dai, et al., Proc. Nat'l Acad. Sci. USA 101:14871-14876 (2004)). The comprehensive transcriptional activities of endothelial cells exposed to these two paradigms of biomechanical stimulation were assessed using genome-wide oligo-microarrays, and further validation of transcription factors selectively upregulated by the athero-protective waveform was performed using real-time PCR. One of the most robustly upregulated transcription factors arising from this analysis was Kruppel-like factor 2 (KLF2), a zinc finger-containing transcription factor previously implicated in vascular development and T cell activation. Several studies have indicated that the expression of KLF2 in cultured endothelial cells is increased after exposure to laminar shear stress (Dekker, et al., Blood 100:1689-1698 (2002); SenBanerjee, J. Exp. Med. 199:1305-1315 (2004)), and Dekker et al. have demonstrated a relative lack of KLF2 expression in the endothelial lining at the bifurcation in the human aorta, a region where early lesions of atherosclerosis are prone to develop (Blood 100:1689-1698 (2002)).

Previous work in our laboratories demonstrated that KLF2 overexpression inhibits the IL-1β-dependent induction of the pro-inflammatory adhesion molecules VCAM-1 and E-selectin in cultured human endothelial cells (SenBanerjee, J. Exp. Med. 199:1305-1315 (2004)). This observation, together with the selective upregulation of KLF2 by an athero-protective waveform and the pattern of expression of KLF2 in the human vasculature, strongly suggested to us that KLF2 might play an important role in the regulation of the flow-mediated atheroprotective endothelial phenotype.

To test this hypothesis, we sought first to define the global transcriptional targets of KLF2 using genome-wide oligo-microarrays on endothelial cells overexpressing GFP or KLF2-GFP in the presence or absence of IL-1β (1 U/ml, 4 h, a well-characterized pro-inflammatory stimulus). A self-organizing map of regulated genes clustered according to similar patterns of expression across the three experimental conditions (KLF2, IL-1, KLF2+IL-1) relative to control (GFP) was developed. These global patterns revealed four distinct clusters of similarly regulated genes. Cluster I represents genes that are down-regulated by KLF2 but are not modulated by IL-1β (e.g., endothelin-1, angiopoietin-2, and endothelial lipase). Cluster II represents genes that are up-regulated by KLF2 and not regulated by IL-1β (e.g., NFATc3, eNOS, and the Nogo receptor). Cluster III includes genes whose up-regulation by IL-1β is antagonized by KLF2 (e.g., IL-6, RANTES, and tissue factor). Finally, cluster IV contains genes that are synergistically upregulated by KLF2 and IL-1β (e.g., ELAFIN, Prostaglandin E Synthase-1, and vitamin-D receptor). These observations coupled with functional categorization of regulated genes (Table 1) indicate that KLF2 serves as a global transcriptional regulator of multiple endothelial functions, including blood vessel development, vascular tone, thrombosis/hemostasis, and inflammation, thus suggesting a central role for KLF2 in the control of endothelial functions critical to the formation and progression of atherosclerotic lesions.

To further assess the role of KLF2 in the regulation of pathophysiologically relevant genes belonging to these four major functional categories, we measured changes in gene expression via real-time Taqman PCR, and also evaluated these changes at the protein level. It was found that KLF2 overexpression robustly increased Tie-2 mRNA and protein levels, while suppressing its autocrine antagonistic ligand, angiopoietin-2 (Ang-2), at the level of mRNA and secreted protein. Tie2, the endothelial-specific receptor for the activating ligand angiopoietin-1, is critical for blood vessel development and stabilization. In contrast, Ang-2 expression leads to vessel destabilization and regression. Furthermore, KLF2 potently upregulated the mRNA expression of elastin and NFATc3, two genes critical for elastogenesis and formation of the arterial wall, respectively (Table 1). These observed effects of KLF2 on genes implicated in the Ang-1/Tie2 axis, elastogenesis, and vessel wall formation may explain the lack of assembly of the vascular tunica media, and vessel wall instability observed in the KLF2 knockout mice (Suri, Cell 87:1171-1180 (1996)).

We also found that KLF2 overexpression upregulated endothelial nitric oxide synthase (eNOS), the enzyme responsible for the production of nitric oxide (NO) in endothelial cells; argininosuccinate synthetase (ASS), a limiting factor in eNOS substrate bioavailability; and dimethylarginine dimethylaminohydrolase 2, an enzyme that degrades an endogenous inhibitor of eNOS. Moreover, KLF2 overexpression decreased levels of caveolin-1 mRNA, a critical negative regulator of eNOS activity.

One of the most strongly upregulated targets of KLF2 is C-type natriuretic peptide (CNP), an endothelial secreted vasodilatory and anti-inflammatory peptide that impinges on the same pathway downstream of NO in adjacent smooth muscle cells. Almost undetectable in endothelial cells in static culture conditions, CNP mRNA and secreted protein levels dramatically increased with KLF2 overexpression. Notably, the expression of endothelin-1 (EDN-1), the most potent known endogenous vasoconstrictor, and angiotensin-converting enzyme (ACE), the cell surface enzyme that produces the vasoconstrictor angiotensin-II were strongly suppressed by KLF2. These data demonstrate an orchestrated upregulation of endothelium-dependent vasodilatory pathways, in particular the L-arginine-NO pathway (a major regulator of vascular tone with critical anti-atherogenic effects), and the downregulation of vasoconstrictive molecules produced by the endothelium.

We next evaluated the KLF2-mediated regulation of genes involved in hemostasis, thrombosis, and inflammation. KLF2 overexpression markedly increased thrombomodulin (TM) mRNA, surface protein expression, and enzymatic activity, as determined by a chromogenic assay for the endothelial cell activation of protein C (aPC). Thrombomodulin, an endothelial cell surface glycoprotein, is a critical cofactor for thrombin-mediated activation of PC, which has potent anti-coagulant properties. Inflammatory stimuli have been shown to induce a pro-coagulant phenotype in endothelial cells. KLF2 expression inhibits the IL-1β-mediated increase of EDG-1 and PAI-1 expression; the former an important receptor for thrombin-mediated pro-coagulant signaling and the latter factor an inhibitor of clot dissolution. Interestingly, we found that KLF2 overexpression dramatically reduced the IL-1β-induction of cell surface tissue factor (TF), the primary cellular initiator of blood coagulation. The coordinated regulation of these critical anti- and pro-coagulant surface proteins suggests that KLF2 expression can confer a robust anti-coagulant endothelial phenotype.

Furthermore, we found that the IL-1β-induction of a large number of pro-inflammatory genes was muted by KLF2. Quantitative Real-time PCR analysis of numerous endothelial genes associated with inflammation confirmed the global suppression by KLF2 of IL-1β mediated endothelial activation first unveiled by microarray analysis (Table 2). These genes encode several cytokines and chemokines that mediate inflammatory cell migration into the vessel wall at sites of physiologic homing or pathological inflammation. Multiplex ELISA was then used to measure the production of various inflammatory cytokines/chemokines in the supernatants from cultured endothelial cells. KLF2 overexpression suppressed the IL-1β-mediated production of IL-6, IL-8, RANTES, IP-10, MCP-1, G-CSF, and GM-CSF. These results strongly support a potential anti-inflammatory effect of KLF2 expression in endothelial cells.

Another one of the highly KLF2-upregulated genes was prostaglandin D2 synthase (PTGDS), which produces as an end-product 15d-PGJ2, the most potent known endogenous ligand of PPAR-γ, a nuclear receptor with multiple anti-inflammatory effects in the vasculature. This nuclear receptor is the target of thiazolidenediones, a widely used class of insulin-sensitizing drugs. The regulation of this pathway is predicted to cooperate with the anti-inflammatory effects observed at the cytokine/chemokine level, and could mediate these effects in an autocrine and/or paracrine fashion. Upregulated KLF2 expression thus globally mutes IL-1β induced endothelial activation as assessed by cytokine and chemokine production, in addition to promoting a potent anti-inflammatory prostaglandin pathway.

In contrast to this IL-1β antagonism, KLF2 and IL-1β synergistically up-regulated various genes involved in the resolution of inflammation (Table 1, Cluster IV). IL-11, a cytokine recently found to have a protective effect on endothelium in an allograft rejection model, was highly expressed only when both KLF2 was expressed and IL-1 was present. The upregulation of the elastase inhibitor ELAFIN also displayed a remarkable synergy between KLF2 and IL-1β (Table 1, Cluster IV), and was found in significant levels only in conditioned media from KLF2-expressing cells exposed to IL-1β. ELAFIN has been shown to potently suppress smooth muscle hyperplasia in animal models of vascular injury and vein graft degeneration. Syndecan 4 (Table 1, Cluster IV) was another synergistically regulated gene; this transmembrane proteoglycan attenuates leukocyte-endothelial adhesion and LPS-mediated systemic shock. The synergistic mode of regulation of these anti-inflammatory targets indicates that KLF2 expression primes the endothelial cell to respond to pro-inflammatory stimuli in order to promote the physiologic resolution of an inflammatory response. Collectively, these data argue for a critical role of KLF2 in attenuating the inflammatory processes that have been causally linked to the initiation and progression of atherosclerotic lesions.

We noted that a large number of genes activated by KLF2 were also regulated in cultured endothelial cells by biomechanical stimulation with the athero-protective waveformn, thus suggesting that the flow-mediated induction of KLF2 might be an important determinant of the atheroprotected endothelial phenotype. Therefore, we compared the genome-wide microarray analyses of genes regulated in cultured human endothelial cells by both the athero-protective shear stress waveform and KLF2 overexpression. Of the genes significantly regulated (p<0.05) by athero-protective shear stress (255 genes), 30% (76 genes) were significantly regulated) of the transcriptional targets regulated by these two stimuli.

To critically assess the role of KLF2 as a mediator of the flow-dependent atheroprotected phenotype, we blocked the flow-induced upregulation of KLF2 using siRNA gene silencing. KLF2 was knocked down in cells exposed to the athero-protective shear stress waveform such that the KFL2 level was approximately equal to that of cells maintained under static control conditions. Importantly, under these experimental conditions we did not observe the activation of the double-stranded RNA-triggered IFN-associated antiviral pathways, as determined by induction of the sensitive marker genes 2′-5′-oligoadenylate synthetase (OAS2) or interferon-inducible transmembrane protein 1 (IFITM1). Blockade of the upregulation of KLF2 in endothelial cells exposed to the athero-protective waveform resulted in a significant loss of the regulation of the most potently upregulated atheroprotective genes, including eNOS, ASS, TM, Tie2, CNP, hPGT, PTGDS, and NOV. The downregulation of IL-8, Ang-2 and ET-1 by the athero-protective waveform was also abolished at the mRNA level. We confirmed these results at the protein level for eNOS and Tie2. The atheroprotective flow-mediated increases in cell surface thrombomodulin expression, secreted levels of CNP, and secreted levels 15-d-PGJ2 were also abolished by blocking the flow-dependent upregulation of KLF2 in a similar fashion (e.g., up- or down-regulated, Table 3). Moreover, of the overlap of genes regulated by both atheroprotective flow and KLF2 overexpression (84 genes), 90% (76 genes) were regulated in the same direction, indicating a strong correlation (p<10-8).

Finally, to determine whether the upregulation of KLF2 and its transcriptional targets are required for the complex functional phenotypes evoked in endothelial cells by athero-protective flow, we utilized assays that assess the responses to an inflammatory or oxidant stress challenge, two critical stimuli causally linked to atherogenesis. First, we examined leukocyte adhesion to endothelial cells challenged with an inflammatory stimulus (IL-1β, 1 U/mL, 6 h). This is a cell-cell interaction critical for the development of early atherosclerotic lesions (Gudi, et al., Proc. Nat'l Acad. Sci. USA 95:2515-2519 (1998)). It was found that preconditioning with the athero-protective shear stress waveform significantly decreased the IL-1β-dependent adhesion of human HL60 cells (a promyelocytic human leukocyte cell line) to endothelial monolayers when compared to static (no flow) controls; blockade of KLF2 upregulation during the shear stress preconditioning suppressed this anti-adhesive phenotype. Second, we tested the role of KLF2 in the resistance to oxidative stress previously observed in endothelial cells preconditioned with shear stress (Hermann, Arterioscler. Thromb. Vasc. Biol. 17:3588-3592 (1997)). KLF2 overexpression was capable of inhibiting H₂O₂-mediated oxidative injury and subsequent cell death as observed by cell morphology in cells cultured under static (no flow) conditions. Preconditioning of endothelial cells with athero-protective shear stress also induced resistance to the oxidant stress challenge, and this resistance was dependent on the upregulation of KLF2. These data demonstrate that KLF2 is necessary and sufficient for the functional phenotypes we observed in endothelial cells exposed to biomechanical stimulation with the athero-protective waveform, and that this transcription factor acts as an integrator of the local humoral and biomechanical milieu.

III. Discussion

It is clear that the function of vascular endothelium is essential to normal vascular physiology, and that its dysfunction can be a critical factor in the pathogenesis of atherosclerosis. Thus, the possibility that hemodynamic forces can act directly as pathophysiologic regulators of endothelial function/dysfunction has provided a conceptual framework for the observation that the earliest lesions of atherosclerosis develop in regions of the arterial tree that correlate with branch points and other regions of disturbed blood flow.

Several studies in vitro have demonstrated that endothelial cells are able to differentially sense and transduce distinct biomechanical stimuli and respond to them with changes in gene expression (Topper, et al, Proc. Nat'l Acad. Sci. USA 93:10417-10422 (1996); Garcia-Cardena, et al., Proc. Nat'l Acad. Sci. USA 98:4478-4485 (2001); McCormick, et al., Proc. Nat'l Acad. Sci. USA 98:8955-8960 (2001); Chien, et al., Biol Bull. 194:390-391; discussion 392-393 (1998); Wasserman, et al., Physiol. Genomics 12:13-23 (2002)). Moreover, endothelial cells acquire specific functional phenotypes depending on the type of biomechanical stimulation to which they are exposed (Dai, et al., Proc, Nat'l Acad. Sci. USA 101:14871-14876 (2004); Garcia-Cardena, et al., Proc. Nat'l Acad. Sci. USA 98:4478-4485 (2001); Brooks, et al., Physiol. Genomics 9:27-41 (2002)). These observations, together with the in vivo demonstration of differential gene expression and activation of signaling pathways in geometrically defined atherosclerosis-prone or atherosclerosis-resistant regions of the mouse or pig aortas (Hajra, et al., Proc. Nat'l Acad. Sci. USA 97:9052-9057 (2000); Passerini, et al., Proc. Nat'l Acad. Sci. USA 101:2482-2487 (2004)) suggests a causative role for hemodynamics in atherogenesis.

Dekker et al. first identified KLF2 as a gene regulated in cultured endothelial cells by steady laminar shear stress and based on its pattern of expression in human arteries these authors suggested a role for this transcription factor in atherogenesis (Dekker, Blood 100:1689-1698 (2002)). Studies from our laboratories demonstrated that overexpression of KLF2 in cultured endothelial cells leads to the inhibition of the IL-1β dependent expression of the adhesion molecules E-selectin and VCAM-1 (SenBanerjee, J. Exp. Med. 199:1305-1315 (2004)). Interestingly, genome-wide transcriptional profiling experiments using cultured endothelial cells overexpressing KLF2 under control or inflammatory conditions revealed that KLF2 regulates transcriptional pathways involved in multiple endothelial functions, including blood vessel formation, control of vasomotor tone, thrombosis, and inflammation, and that this regulation confers an atheroprotective endothelial phenotype.

Two major pathophysiological processes that play a critical role in early and late stages of atherosclerosis are inflammation and thrombosis. Our data document a global anti-inflammatory and anti-thrombotic role for KLF2 in endothelial cells. Overexpression of KLF2 not only blocks the pro-thrombotic induction of tissue factor by an inflammatory stimulus (e.g., IL-1β), but also potently induces expression of a well-known endothelial anti-thrombotic cell surface molecule, thrombomodulin. The upregulation of thrombomodulin expression by shear stress has been previously demonstrated in vitro and in vivo (Yamashita, et al, Circulation 108:2450-2452 (2003); Takada, et al., Biochem. Biophys. Res. Commun. 205:1345-1352 (1994)). Here we demonstrate KLF2 as a regulator of thrombomodulin in an atheroprotective hemodynamic environment.

The global anti-inflammatory effects of KLF2 expression manifest as a coordinated regulation of multiple inflammatory mediators at the gene expression and secreted protein level. How KLF2 is able to silence such a large spectrum of the pro-inflammatory phenotype is still unclear, but is reminiscent of its observed effects on T cell activation, which also represents a global phenotypic switch (Kuo, et al., Science 277:1986-1990 (1997)). Importantly, the relationship between KLF2 and the pro-inflammatory stimulus is not a simple antagonism. In our transcriptional profiling analysis, we observed a group of genes that displayed a marked synergistic regulation by KLF2 and IL-1β. Among these genes were ELAFIN/SKALP, syndecan 4, and IL-11, anti-inflammatory molecules that play an important role in the resolution of inflammation would be predicted to attenuate the pathological processes in atherosclerosis. Thus, KLF2 has the ability to not only prevent initiation of inflammatory activation in endothelial cells, but also primes these cells to respond to inflammatory insults and efficiently promote rapid resolution of the inflammatory process.

Here we demonstrate that KLF2 is upregulated by an athero-protective shear stress waveform but not by an athero-prone waveform. Interestingly, analysis of the genome-wide transcriptional profiles evoked by atheroprotective flow and overexpression of KLF2 revealed that 30% of the genes significantly regulated in both conditions overlap, and that 90% of these genes were regulated in the same direction (up or down). This remarkable correlation supports the hypothesis that KLF2 is a key and important mediator of the atheroprotective phenotype evoked by atheroprotective flow. Using RNAi silencing of KLF2 under atheroprotective flow we were able to demonstrate that the expression of several genes regulated by the atheroprotective waveform is dependent on KLF2.

### Legends to Tables

Table 1: Whole-genome expression patterns controlled by KLF2 and/or IL-1 in HUVEC were visualized. HUVEC were infected with either Ad-GFP or Ad-KLF2 and incubated for 24 hours when RNA was isolated and analyzed using whole genome microarrays. Self-organizing map (SOM) software was used to cluster similarly regulated genes based on intensity of expression relative to GFP-expressing (control) cells. Table 1 shows selected KLF2-responsive genes grouped by clusters, with major associated biological functions.

Table 2: Table 2 shows the effect of KLF2 and/or IL-1 on inflammatory gene expression.

Table 3: Genes significantly regulated in the same direction by both KLF2 and athero-protective waveform (76 Genes). Genes with the same direction of regulation by both KLF2 and athero-protective waveform are displayed. * In the presence of Ad-KLF2 and IL-1β as compared to Ad-GFP and IL-1β (attenuation). ** In the presence of Ad-KLF2 and IL-1β as compared to Ad-KLF2 and as compared to IL-1β (synergism).

Table 4: Regulatory genes induced by shear stress. t,0250

TABLE 2

| | | | Ad-GFP + | AD-KLF2 + |
|---|---|---|---|---|
| | Ad-GFP | Ad-KLF2 | IL-1 | IL-1 |
| IL-6 | 1.0 | −1.8(.3) | 144.8(63.5) | 24.3(7.4) |
| IL-8 | 1.0 | −4.1(.1) | 206.4(107.1) | 31.5(11.3) |
| MCP-1 | 1.0 | −2.6(.2) | 140.9(78.7) | 19.9(1.5) |
| E-selectin | 1.0 | −3.47(.1) | 122.3(59.4) | 25.7(8.8) |
| TNF | 1.0 | 1.5(.9) | 246.9(75.8) | 85.6(65.1) |
| CXCL10/IP-10 | 1.0 | −1.4(.4) | 674.7(62.5) | 62.8(28.9) |
| CXCL11/I-TAC | 1.0 | −1.8(.4) | 22.4(16.3) | 1.9(.8) |
| IFN-g | 1.0 | −2.6(.12) | 143.9(28.4) | 23.9(9.5) |
| COX-2 | 1.0 | −1.2(.2) | 16.8(5.2) | 1.8(1.4) |
| CCL5 | 1.0 | 1.2(.2) | 34.7(6.5) | 11.7(2.9) |
| IL-15 | 1.0 | 2.1(.7) | 18.2(1.4) | 5.1(.9) |
| IL-1a | 1.0 | 5.7(1.4) | 55.2(15.7) | 15.5(6.9) |
| IL-1b | 1.0 | 16.8(11.1) | 73.7(8.9) | 34.3(16.3) |

TABLE 3

| Response | | | |
|---|---|---|---|
| to Flow | Reference | | |
| Induced | Sequence | | SEQ |
| KLF2 | Number | Description | ID NO: |
| ↑ | NM_005980 | Homo sapiens S100 calcium | 1 |
| | | binding protein P (S100P), | |
| | | mRNA | |
| ↑ | NM_005630 | Homo sapiens solute carrier | 2 |
| | | organic anion transporter family, | |
| | | member 2A1 (SLCO2A1), | |
| | | mRNA | |
| ↑ | NM_198946 | Homo sapiens lipocalin 6 | 3 |
| | | (LCN6), mRNA | |
| ↑ | NM_003665 | Homo sapiens ficolin | 4 |
| | | (collagen/fibrinogen domain | |
| | | containing) 3 (Hakata antigen) | |
| | | (FCN3), transcript variant 1, | |
| | | mRNA | |
| ↑ | NM_002820 | Homo sapiens parathyroid | 5 |
| | | hormone-like hormone (PTHLH), | |
| | | transcript variant 2, mRNA | |
| ↑ | NM_033649 | Homo sapiens fibroblast growth | 6 |
| | | factor 18 (FGF18), transcript | |
| | | variant 2, mRNA | |
| ↑ | NM_002960 | Homo sapiens S100 calcium | 7 |
| | | binding protein A3 (S100A3), | |
| | | mRNA | |
| ↑ | NM_173652 | Homo sapiens hypothetical | 8 |
| | | protein MGC34824 | |
| | | (MGC34824), mRNA | |
| ↑ | NM_000050 | Homo sapiens argininosuccinate | 9 |
| | | synthetase (ASS), transcript | |
| | | variant 1, mRNA | |
| ↑ | XM_380034 | PREDICTED: Homo sapiens | 10 |
| | | similar to Argininosuccinate | |
| | | synthase (Citrulline-aspartate | |
| | | ligase) (LOC402687), mRNA | |
| ↑ | NM_004695 | Homo sapiens solute carrier | 11 |
| | | family 16 (monocarboxylic acid | |
| | | transporters), member 5 | |
| | | (SLC16A5), mRNA | |
| ↑ | NM_014147 | Homo sapiens HSPC047 protein | 12 |
| | | (HSPC047), mRNA | |
| ↑ | NM_018421 | Homo sapiens TBC1 domain | 13 |
| | | family, member 2 (TBC1D2), | |
| | | mRNA | |
| ↑ | NM_005358 | Homo sapiens LIM domain 7 | 14 |
| | | (LMO7), mRNA | |
| ↑ | NM_025214 | Homo sapiens CTCL tumor | 15 |
| | | antigen se57-1 (SE57-1), mRNA | |
| ↑ | NM_001425 | Homo sapiens epithelial | 16 |
| | | membrane protein 3 (EMP3), | |
| | | mRNA | |
| ↑ | NM_052970 | Homo sapiens heat shock 70 kD | 17 |
| | | protein 12B (HSPA12B), mRNA | |
| ↑ | NM_020467 | Homo sapiens hypothetical | 18 |
| | | protein from clone 643 | |
| | | (LOC57228), mRNA | |
| ↑ | NM_000304 | Homo sapiens peripheral myelin | 19 |
| | | protein 22 (PMP22), transcript | |
| | | variant 1, mRNA | |
| ↑ | NM_002430 | Homo sapiens meningioma | 20 |
| | | (disrupted in balanced | |
| | | translocation) 1 (MN1), mRNA | |
| ↑ | NM_000201 | Homo sapiens intercellular | 21 |
| | | adhesion molecule 1 (CD54), | |
| | | human rhinovirus receptor | |
| | | (ICAM1), mRNA | |
| ↑ | NM_001505 | Homo sapiens G protein-coupled | 22 |
| | | receptor 30 (GPR30), transcript | |
| | | variant 2, mRNA | |
| ↑ | XM_166090 | PREDICTED: Homo sapiens | 23 |
| | | placenta-specific 9 (PLAC9), | |
| | | mRNA | |
| ↑ | NM_001312 | Homo sapiens cysteine-rich | 24 |
| | | protein 2 (CRIP2), mRNA | |
| ↑ | NM_178507 | Homo sapiens NS5ATP13TP2 | 25 |
| | | protein (NS5ATP13TP2), mRNA | |
| ↑ | NM_000459 | Homo sapiens TEK tyrosine | 26 |
| | | kinase, endothelial (venous | |
| | | malformations, multiple | |
| | | cutaneous and mucosal) (TEK), | |
| | | mRNA | |
| ↑ | NM_004785 | Homo sapiens solute carrier | 27 |
| | | family 9 (sodium/hydrogen | |
| | | exchanger), member 3 regulator 2 | |
| | | (SLC9A3R2), mRNA | |
| ↑ | NM_006096 | Homo sapiens N-myc | 28 |
| | | downstream regulated gene 1 | |
| | | (NDRG1), mRNA | |
| ↑ | NM_001779 | Homo sapiens CD58 antigen, | 29 |
| | | (lymphocyte function-associated | |
| | | antigen 3) (CD58), mRNA | |
| ↑ | NM_015621 | Homo sapiens DKFZP434C171 | 30 |
| | | protein (DKFZP434C171), | |
| | | mRNA | |
| ↑ | NM_018286 | Homo sapiens hypothetical | 31 |
| | | protein FLJ10970 (FLJ10970), | |
| | | mRNA | |
| ↑ | NM_022152 | Homo sapiens transmembrane | 32 |
| | | BAX inhibitor motif containing 1 | |
| | | (TMBIM1), mRNA | |
| ↑ | NM_005854 | Homo sapiens receptor | 33 |
| | | (calcitonin) activity modifying | |
| | | protein 2 (RAMP2), mRNA | |
| ↑ | NM_032823 | Homo sapiens chromosome 9 | 34 |
| | | open reading frame 3 (C9orf3), | |
| | | mRNA | |
| ↑ | NM_002224 | Homo sapiens inositol 1,4,5- | 35 |
| | | triphosphate receptor, type 3 | |
| | | (ITPR3), mRNA | |
| ↑ | NM_139247 | Homo sapiens adenylate cyclase | 36 |
| | | 4 (ADCY4), mRNA | |
| ↑ | NM_032227 | Homo sapiens hypothetical | 37 |
| | | protein FLJ22679 (RP13- | |
| | | 360B22.2), mRNA | |
| ↑ | NM_024496 | Homo sapiens chromosome 14 | 38 |
| | | open reading frame 4 (C14orf4), | |
| | | mRNA | |
| ↑ | NM_005397 | Homo sapiens podocalyxin-like | 39 |
| | | (PODXL), transcript variant 2, | |
| | | mRNA | |
| ↑ | NM_002047 | Homo sapiens glycyl-tRNA | 40 |
| | | synthetase (GARS), mRNA | |
| ↑ | NM_002837 | Homo sapiens protein tyrosine | 41 |
| | | phosphatase, receptor type, B | |
| | | (PTPRB), mRNA | |
| ↑ | NM_181353 | Homo sapiens inhibitor of DNA | 42 |
| | | binding 1, dominant negative | |
| | | helix-loop-helix protein (ID1), | |
| | | transcript variant 2, mRNA | |
| ↑ | NM_000611 | Homo sapiens CD59 antigen | 43 |
| | | p18-20 (antigen identified by | |
| | | monoclonal antibodies 16.3A5, | |
| | | EJ16, EJ30, EL32 and G344) | |
| | | (CD59), transcript variant 2, | |
| | | mRNA | |
| ↑ | NM_005587 | Homo sapiens MADS box | 44 |
| | | transcription enhancer factor 2, | |
| | | polypeptide A (myocyte enhancer | |
| | | factor 2A) (MEF2A), mRNA | |
| ↑ | NM_001423 | Homo sapiens epithelial | 45 |
| | | membrane protein 1 (EMP1), | |
| | | mRNA | |
| ↑ | NM_006870 | Homo sapiens destrin (actin | 46 |
| | | depolymerizing factor) (DSTN), | |
| | | transcript variant 1, mRNA | |
| ↑ | NM_002317 | Homo sapiens lysyl oxidase | 47 |
| | | (LOX), mRNA | |
| ↑ | NM_023004 | Homo sapiens reticulon 4 | 48 |
| | | receptor (RTN4R), mRNA | |
| ↑ | NM_024409 | Homo sapiens natriuretic peptide | 49 |
| | | precursor C (NPPC), mRNA | |
| ↑ | NM_000603 | Homo sapiens nitric oxide | 50 |
| | | synthase 3 (endothelial cell) | |
| | | (NOS3), mRNA | |
| ↑ | NM_000954 | Homo sapiens prostaglandin D2 | 51 |
| | | synthase 21 kDa (brain) | |
| | | (PTGDS), mRNA | |
| ↑ | NM_173163 | Homo sapiens nuclear factor of | 52 |
| | | activated T-cells, cytoplasmic, | |
| | | calcineurin-dependent 3 | |
| | | (NFATC3), transcript variant 3, | |
| | | mRNA | |
| ↓ | NM_001753 | Homo sapiens caveolin 1, | 53 |
| | | caveolae protein, 22 kDa (CAV1), | |
| | | mRNA | |
| ↓ | NM_004105 | Homo sapiens EGF-containing | 54 |
| | | fibulin-like extracellular matrix | |
| | | protein 1 (EFEMP1), transcript | |
| | | variant 1, mRNA | |
| ↓ | NM_020749 | Homo sapiens mitochondrial | 55 |
| | | tumor suppressor 1 (MTUS1), | |
| | | nuclear gene encoding | |
| | | mitochondrial protein, transcript | |
| | | variant 5, mRNA | |
| ↓ | NM_005694 | Homo sapiens COX17 homolog, | 56 |
| | | cytochrome c oxidase assembly | |
| | | protein (yeast) (COX17), nuclear | |
| | | gene encoding mitochondrial | |
| | | protein, mRNA | |
| ↓ | NM_004772 | Homo sapiens chromosome 5 | 57 |
| | | open reading frame 13 (C5orf13), | |
| | | mRNA | |
| ↓ | NM_032962 | Homo sapiens chemokine (C—C | 58 |
| | | motif) ligand 14 (CCL14), | |
| | | transcript variant 2, mRNA | |
| ↓ | NM_006452 | Homo sapiens | 59 |
| | | phosphoribosylaminoimidazole | |
| | | carboxylase, | |
| | | phosphoribosylaminoimidazole | |
| | | succinocarboxamide synthetase | |
| | | (PAICS), mRNA | |
| ↓ | NM_006528 | Homo sapiens tissue factor | 60 |
| | | pathway inhibitor 2 (TFPI2), | |
| | | mRNA | |
| ↓ | NM_014452 | Homo sapiens tumor necrosis | 61 |
| | | factor receptor superfamily, | |
| | | member 21 (TNFRSF21), mRNA | |
| ↓ | NM_015854 | Homo sapiens retinoic acid | 62 |
| | | receptor-beta associated open | |
| | | reading frame (LOC51036), | |
| | | mRNA | |
| ↓ | NM_000689 | Homo sapiens aldehyde | 63 |
| | | dehydrogenase 1 family, member | |
| | | A1 (ALDH1A1), mRNA | |
| ↓ | NM_139266 | Homo sapiens signal transducer | 64 |
| | | and activator of transcription 1, | |
| | | 91 kDa (STAT1), transcript | |
| | | variant beta, mRNA | |
| ↓ | NM_014988 | Homo sapiens KIAA1102 protein | 65 |
| | | (KIAA1102), mRNA | |
| ↓ | NM_002970 | Homo sapiens | 66 |
| | | spermidine/spermine N1- | |
| | | acetyltransferase (SAT), mRNA | |
| ↓ | NM_138735 | Homo sapiens neurexin 1 | 67 |
| | | (NRXN1), transcript variant beta, | |
| | | mRNA | |
| ↓ | NM_018371 | Homo sapiens chondroitin | 68 |
| | | beta1,4 N- | |
| | | acetylgalactosaminyltransferase | |
| | | (ChGn), mRNA | |
| ↓ | NM_170773 | Homo sapiens Ras association | 69 |
| | | (RalGDS/AF-6) domain family 2 | |
| | | (RASSF2), transcript variant 2, | |
| | | mRNA | |
| ↓ | NM_003199 | Homo sapiens transcription factor | 70 |
| | | 4 (TCF4), mRNA | |
| ↓ | NM_001444 | Homo sapiens fatty acid binding | 71 |
| | | protein 5 (psoriasis-associated) | |
| | | (FABP5), mRNA | |
| ↓ | XM_379976 | PREDICTED: Homo sapiens | 72 |
| | | similar to Fatty acid-binding | |
| | | protein, epidermal (E-FABP) | |
| | | (Psoriasis-associated fatty acid- | |
| | | binding protein homolog) (PA- | |
| | | FABP) (LOC402628), mRNA | |
| ↓ | NM_015068 | Homo sapiens paternally | 73 |
| | | expressed 10 (PEG10), mRNA | |
| ↓ | NM_005824 | Homo sapiens leucine rich repeat | 74 |
| | | containing 17 (LRRC17), | |
| | | transcript variant 2, mRNA | |
| ↓ | XM_370729 | PREDICTED: Homo sapiens | 75 |
| | | similar to Fatty acid-binding | |
| | | protein, epidermal (E-FABP) | |
| | | (Psoriasis-associated fatty acid- | |
| | | binding protein homolog) (PA- | |
| | | FABP) (LOC387934), mRNA | |
| ↓ | NM_005118 | Homo sapiens tumor necrosis | 76 |
| | | factor (ligand) superfamily, | |
| | | member 15 (TNFSF15), mRNA | |
| ↓ | NM_015687 | Homo sapiens filamin A | 77 |
| | | interacting protein 1 (FILIP1), | |
| | | mRNA | |
| ↓ | NM_001442 | Homo sapiens fatty acid binding | 78 |
| | | protein 4, adipocyte (FABP4), | |
| | | mRNA | |
| ↓ | NM_001147 | Homo sapiens angiopoietin 2 | 79 |
| | | (ANGPT2), mRNA | |
| ↓ | NM_001718 | Homo sapiens bone | 80 |
| | | morphogenetic protein 6 | |
| | | (BMP6), mRNA | |
| ↓ | NM_001955 | Homo sapiens endothelin 1 | 81 |
| | | (EDN1), mRNA | |
| ↓ | NM_005242 | Homo sapiens coagulation factor | 82 |
| | | II (thrombin) receptor-like 1 | |
| | | (F2RL1), mRNA | |
| ↓ | NM_006033 | Homo sapiens lipase, endothelial | 83 |
| | | (LIPG), mRNA | |
| ↓* | NM_001964 | Homo sapiens early growth | 84 |
| | | response 1 (EGR1), mRNA | |
| ↓* | NM_000600 | Homo sapiens interleukin 6 | 85 |
| | | (interferon, beta 2) (IL6), mRNA | |
| ↓* | NM_005409 | Homo sapiens chemokine (C—X—C | 86 |
| | | motif) ligand 11 (CXCL11), | |
| | | mRNA | |
| ↓* | NM_002985 | Homo sapiens chemokine (C—C | 87 |
| | | motif) ligand 5 (CCL5), mRNA | |
| ↓* | NM_001078 | Homo sapiens vascular cell | 88 |
| | | adhesion molecule 1 (VCAM1), | |
| | | transcript variant 1, mRNA | |
| ↓* | NM_002089 | Homo sapiens chemokine (C—X—C | 89 |
| | | motif) ligand 2 (CXCL2), | |
| | | mRNA | |
| ↓* | NM_002994 | Homo sapiens chemokine (C—X—C | 90 |
| | | motif) ligand 5 (CXCL5), | |
| | | mRNA | |
| ↓* | NM_001993 | Homo sapiens coagulation factor | 91 |
| | | III (thromboplastin, tissue factor) | |
| | | (F3), mRNA | |
| ↑** | NM_002638 | Homo sapiens peptidase inhibitor | 92 |
| | | 3, skin-derived (SKALP) (PI3), | |
| | | mRNA | |
| ↑** | NM_003376 | Homo sapiens vascular | 93 |
| | | endothelial growth factor | |
| | | (VEGF), transcript variant 2, | |
| | | mRNA | |
| ↑** | NM_002607 | Homo sapiens platelet-derived | 94 |
| | | growth factor alpha polypeptide | |
| | | (PDGFA), transcript variant 1, | |
| | | mRNA | |
| ↑** | NM_002608 | Homo sapiens platelet-derived | 95 |
| | | growth factor beta polypeptide | |
| | | (simian sarcoma viral (v-sis) | |
| | | oncogene homolog) (PDGFB), | |
| | | transcript variant 1, mRNA | |
| ↑** | NM_004878 | Homo sapiens prostaglandin E | 96 |
| | | synthase (PTGES), transcript | |
| | | variant 1, mRNA | |
| ↑** | NM_002999 | Homo sapiens syndecan 4 | 97 |
| | | (amphiglycan, ryudocan) | |
| | | (SDC4), mRNA | |
| ↑** | NM_000376 | Homo sapiens vitamin D (1,25- | 98 |
| | | dihydroxyvitamin D3) receptor | |
| | | (VDR), transcript variant 1, | |
| | | mRNA | |

TABLE 4

| Reference | | |
|---|---|---|
| Sequence | | |
| Number | Description | SEQ ID NO: |
| NM_002401 | Homo sapiens mitogen-activated | 99 |
| | protein kinase kinase kinase 3 | |
| | (MAP3K3), transcript variant 2, mRNA | |
| NM_002757 | Homo sapiens mitogen-activated | 100 |
| | protein kinase kinase 5 (MAP2K5), | |
| | transcript variant B, mRNA | |
| NM_002749 | Homo sapiens mitogen-activated | 101 |
| | protein kinase 7 (MAPK7), transcript | |
| | variant 3, mRNA | |
| NM_005587 | Homo sapiens MADS box transcription | 102 |
| | enhancer factor 2, polypeptide A | |
| | (myocyte enhancer factor 2A) | |
| | (MEF2A), mRNA | |
| NM_002397 | Homo sapiens MADS box transcription | 103 |
| | enhancer factor 2, polypeptide C | |
| | (myocyte enhancer factor 2C) | |
| | (MEF2C), mRNA | |
| NM_016270 | Homo sapiens Kruppel-like factor 2 | 104 |
| | (lung) (KLF2), mRNA | |

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

1. A method of examining a subject for the presence of endothelial cells protected or prone to the development of an atherosclerotic plaque, comprising:
a) assaying a test sample of blood, serum or plasma from said subject to determine the level of at least one marker gene sequence selected from the group consisting of SEQ ID NO:1-SEQ ID NO:103;
b) comparing the results obtained from the assay of step a) with results from one or more control samples; and
c) concluding that said subject is at increased risk of atherosclerotic plaque formation based upon whether the level of said marker gene sequence in said test sample of blood, serum or plasma is higher or lower than in said control sample.

2. The method of claim 1, wherein:
a) said marker gene sequence is selected from the group consisting of SEQ ID NO:1-SEQ ID NO:52;
b) said control samples reflect the level of said marker gene sequence when only healthy (atherosclerosis-resistant) endothelial cells are present or said control samples are representative of the population as a whole; and
c) it is concluded that said subject has endothelial cells prone to atherosclerotic plaque formation if the level of said marker gene sequence in said test sample is lower than in said controls.

3. The method of claim 2, wherein said marker gene sequence is for natriuretic peptide precursor C.

4. The method of claim 1, wherein:
a) said marker gene sequence is selected from the group consisting of SEQ ID NO:53-SEQ ID NO:83;
b) said control samples reflect the level of said marker gene sequence detected when only healthy (atherosclerosis-resistant) endothelial cells are present or said control samples are representative of the population as a whole; and
c) it is concluded that said subject has endothelial cells prone to atherosclerotic plaque formation if the level of said marker gene sequence in said test sample is higher than in said control samples.

5. A method of determining whether test endothelial cells are in a healthy (atherosclerosis-resistant) state or a pathological (atherosclerosis-prone) state, comprising:
a) assaying the level of a marker gene sequence in said endothelial cells, said marker gene sequence being selected from the group consisting of SEQ ID NO:1-SEQ ID NO:103;
b) comparing the results obtained in step a) with results from similar assays performed using control endothelial cells; and
c) concluding that said test endothelial cells are in a healthy state if the level of said marker gene sequence in said test sample of blood, serum or plasma is significantly higher or lower than in said control samples.

6. The method claim 5, wherein said method further comprises determining the level of the KLF2 gene sequence (SEQ ID NO:104) in said endothelial cells and comparing the results obtained with the amount of KLF2 in control endothelial cells.

7. The method of claim 5, wherein assays of said marker gene sequence comprise amplifying the mRNA present in said endothelial cells using the polymerase chain reaction (PCR).

8. The method of claim 5, wherein said assay of said marker gene sequence is a microarray assay.

9. The method of claim 5, wherein:
a) said marker gene sequence is selected from the group consisting of SEQ ID NO:1-SEQ ID NO:103;
b) said control samples reflect the level of said marker gene sequence in healthy (atherosclerosis-resistant) endothelial cells or said control samples are representative of the average level of said marker gene sequence in endothelial cells from a population of subjects; and
c) it is concluded that endothelial cells are atherosclerosis-prone if the level of said marker gene sequence in said test sample is lower than in said control samples.

10. The method of claim 9, further comprising comparing the level of the KLF2 gene sequence (SEQ ID NO104) in said test endothelial cells with the level of KLF2 gene sequence in said control endothelial cells.

11. The method of claim 9, wherein the assay of said marker gene sequence comprises amplifying the mRNA present in said test endothelial cells using PCR.

12. The method of claim 9, wherein the assay of said marker gene sequence is a microarray assay.

13. The method of claim 5, wherein:
a) said marker gene sequence is selected from the group consisting of SEQ ID NO:53-SEQ ID NO:83;
b) said control cells reflect the level of said marker gene sequence in healthy (atherosclerosis-resistant) endothelial cells or said control samples are representative of the average level of said marker gene sequence in endothelial cells from a population of subjects; and
c) it is concluded that said endothelial cells are prone to atherosclerosis if the level of said marker gene sequence in said test sample is higher than in said control samples.

14. The method of claim 13, further comprising comparing the level of KLF2 gene sequence (SEQ ID NO:104) in said test endothelial cells with the level of KLF2 gene sequence in said control endothelial cells.

15. The method of claim 13, wherein said assay comprises PCR amplifying the mRNA present in said test endothelial cells.

16. The method of claim 13, wherein said assay is a microarray assay.

17-34. (canceled)

35. A microarray plate comprising a series of distinct immobilized oligonucleotides, wherein:
a) at least one of said oligonucleotides hybridizes under stringent conditions specifically to a gene sequence selected from SEQ ID NO:1-SEQ ID NO:103;
b) said oligonucleotides that hybridize under stringent conditions specifically to said gene sequence are immobilized at a location on said microarray plate that does not contain any oligonucleotides that hybridize to other sequences under stringent conditions; and
c) said microarray plate contains no more than 500 distinct immobilized oligonucleotides in total.

36. The microarray plate of claim 35, wherein said plate has at least 10 distinct oligonucleotides hybridizing under stringent conditions to a gene sequence selected from the group consisting of SEQ ID NO:1-SEQ ID NO:103 and wherein said microarray plate contains no more than 200 distinct immobilized oligonucleotides in total.

37. The microarray plate of claim 36, wherein said plate has at least 30 distinct oligonucleotides hybridizing under stringent conditions to a marker gene sequence selected from the group consisting of SEQ ID NO:1-SEQ ID NO:103.

38. The microarry plate of claim 36, wherein said microarray plate has at least 50 distinct oligonucleotides hybridizing under stringent conditions to a marker gene sequence selected from the group consisting of SEQ ID NO:1-SEQ ID NO:103.

39. The microarry plate of claim 36, wherein said plate has distinct oligonucleotides hybridizing under stringent conditions to each of the marker gene sequence of SEQ ID NO:1-SEQ ID NO:103.

40. The microarry plate of claim 39, wherein said microarray plate contains no more than 100 distinct immobilized oligonucleotides in total.

41. The microarry plate of claim 35, further comprising a distinct immobilized oligonucleotide that hybridizes under stringent conditions to a nucleic acid encoding KLF2 (SEQ ID NO:104).
